SEQUENCE LISTING (17 sequences)

SEQ ID NO: 1 - protein, 852 aa, Homo sapiens
MKRAAAKHLIERYYHQLTEGCGNEACTNEFCASCPTFLRMDNNAAAIKALELYKINAKLCDPHPSKKGASSAYLENSKGAPNNSCSEIKMNKKGARIDFKDVTYLTEEKVYEILELCREREDYSPLIRVIGRVFSSAEALVQSFRKVKQHTKEELKSLQAKDEDKDEDEKEKAACSAAAMEEDSEASSSRIGDSSQGDNNLQKLGPDDVSVDIDAIRRVYTRLLSNEKIETAFLNALVYLSPNVECDLTYHNVYSRDPNYLNLFIIVMENRNLHSPEYLEMALPLFCKAMSKLPLAAQGKLIRLWSKYNADQIRRMMETFQQLITYKVISNEFNSRNLVNDDDAIVAASKCLKMVYYANVVGGEVDTNHNEEDDEEPIPESSELTLQELLGEERRNKKGPRVDPLETELGVKTLDCRKPLIPFEEFINEPLNEVLEMDKDYTFFKVETENKFSFMTCPFILNAVTKNLGLYYDNRIRMYSERRITVLYSLVQGQQLNPYLRLKVRRDHIIDDALVRLEMIAMENPADLKKQLYVEFEGEQGVDEGGVSKEFFQLVVEEIFNPDIGMFTYDESTKLFWFNPSSFETEGQFTLIGIVLGLAIYNNCILDVHFPMVVYRKLMGKKGTFRDLGDSHPVLYQSLKDLLEYEGNVEDDMMITFQISQTDLFGNPMMYDLKENGDKIPITNENRKEFVNLYSDYILNKSVEKQFKAFRRGFHMVTNESPLKYLFRPEEIELLICGSRNLDFQALEETTEYDGGYTRDSVLIREFWEIVHSFTDEQKRLFLQFTTGTDRAPVGGLGKLKMIIAKNGPDTERLPTSHTCFNVLLLPEYSSKEKLKERLLKAITYAKGFGML

SEQ ID NO: 2 - protein, 875 aa, Homo sapiens
MEKLHQCYWKSGEPQSDDIEASRMKRAAAKHLIERYYHQLTEGCGNEACTNEFCASCPTFLRMDNNAAAIKALELYKINAKLCDPHPSKKGASSAYLENSKGAPNNSCSEIKMNKKGARIDFKDVTYLTEEKVYEILELCREREDYSPLIRVIGRVFSSAEALVQSFRKVKQHTKEELKSLQAKDEDKDEDEKEKAACSAAAMEEDSEASSSRIGDSSQGDNNLQKLGPDDVSVDIDAIRRVYTRLLSNEKIETAFLNALVYLSPNVECDLTYHNVYSRDPNYLNLFIIVMENRNLHSPEYLEMALPLFCKAMSKLPLAAQGKLIRLWSKYNADQIRRMMETFQQLITYKVISNEFNSRNLVNDDDAIVAASKCLKMVYYANVVGGEVDTNHNEEDDEEPIPESSELTLQELLGEERRNKKGPRVDPLETELGVKTLDCRKPLIPFEEFINEPLNEVLEMDKDYTFFKVETENKFSFMTCPFILNAVTKNLGLYYDNRIRMYSERRITVLYSLVQGQQLNPYLRLKVRRDHIIDDALVRLEMIAMENPADLKKQLYVEFEGEQGVDEGGVSKEFFQLVVEEIFNPDIGMFTYDESTKLFWFNPSSFETEGQFTLIGIVLGLAIYNNCILDVHFPMVVYRKLMGKKGTFRDLGDSHPVLYQSLKDLLEYEGNVEDDMMITFQISQTDLFGNPMMYDLKENGDKIPITNENRKEFVNLYSDYILNKSVEKQFKAFRRGFHMVTNESPLKYLFRPEEIELLICGSRNLDFQALEETTEYDGGYTRDSVLIREFWEIVHSFTDEQKRLFLQFTTGTDRAPVGGLGKLKMIIAKNGPDTERLPTSHTCFNVLLLPEYSSKEKLKERLLKAITYAKGFGML

SEQ ID NO: 3 - protein, 872 aa, Homo sapiens
MATACKRSGEPQSDDIEASRMKRAAAKHLIERYYHQLTEGCGNEACTNEFCASCPTFLRMDNNAAAIKALELYKINAKLCDPHPSKKGASSAYLENSKGAPNNSCSEIKMNKKGARIDFKDVTYLTEEKVYEILELCREREDYSPLIRVIGRVFSSAEALVQSFRKVKQHTKEELKSLQAKDEDKDEDEKEKAACSAAAMEEDSEASSSRIGDSSQGDNNLQKLGPDDVSVDIDAIRRVYTRLLSNEKIETAFLNALVYLSPNVECDLTYHNVYSRDPNYLNLFIIVMENRNLHSPEYLEMALPLFCKAMSKLPLAAQGKLIRLWSKYNADQIRRMMETFQQLITYKVISNEFNSRNLVNDDDAIVAASKCLKMVYYANVVGGEVDTNHNEEDDEEPIPESSELTLQELLGEERRNKKGPRVDPLETELGVKTLDCRKPLIPFEEFINEPLNEVLEMDKDYTFFKVETENKFSFMTCPFILNAVTKNLGLYYDNRIRMYSERRITVLYSLVQGQQLNPYLRLKVRRDHIIDDALVRLEMIAMENPADLKKQLYVEFEGEQGVDEGGVSKEFFQLVVEEIFNPDIGMFTYDESTKLFWFNPSSFETEGQFTLIGIVLGLAIYNNCILDVHFPMVVYRKLMGKKGTFRDLGDSHPVLYQSLKDLLEYEGNVEDDMMITFQISQTDLFGNPMMYDLKENGDKIPITNENRKEFVNLYSDYILNKSVEKQFKAFRRGFHMVTNESPLKYLFRPEEIELLICGSRNLDFQALEETTEYDGGYTRDSVLIREFWEIVHSFTDEQKRLFLQFTTGTDRAPVGGLGKLKMIIAKNGPDTERLPTSHTCFNVLLLPEYSSKEKLKERLLKAITYAKGFGML

SEQ ID NO: 4 - DNA, 2865 bp, Homo sapiens
  source 1..2865: mol_type = other DNA
cctgctctctgtgtgggaggtgttgtgtggggcatggatagggacccaccagagacaacacatggccctcgggccccaggctgcaatttctcagtgcatgcttggagacggtcctgggcctcctccatgcccagggtcaggctgtgcaactgtcaggttagcggcggcatgtggtgtctgggcctgtctacttcgttgtccgtgagctgctggtggccctagccctttcccaggggttcaggtgcagccctggggttgtgtgggacagccagtgtgggcagtggagctgctctggaggcctgctttagggtgcagcctgggtgtcttgcccgagtgtagtgacccgcctgccgtgtcctcacggggttccctagcaggaagggccaagggtcccctgtgtctgtgacacgtgagtgtttcccatgggtcccatcaaatgggtccacggagggagagcatgagtgaactggtaccccccacccccaccctcttccccacctggaggcatgagcccctggttggacatatgtcctcctcctgtacccccagatggcagcgctggaggaggacttgtactggggccgctggccccacatcagtgtccgtcatcccggcgtccagcccctggtgtgctgaagctcaggcccttccttgtgccctagtctcctgcactgagcttggtgagcccattcggatactgctggatgcatgggtcgggagggaggtgccctgggttgggtcgatgatgagaaccttatattgtcctgaagagaggtgatgacttaaaaatcattctcaaaaggattatgctgaggcccggcctaggttagaattttggaagaggatgctgggatcctgaggtccccaggagagcccctttatttttggctggagacctggaggccctgaagggtatctggggggcccagccctgtccctatactcctctgtgaggcagcccaggctctctgtgtgggcttggtgttggcactctggttttctgggggcggttcaggttgctggttccttctgaaccattgttctccccttgattctttcagtgagctcttctgctcaggcgggtcccctggcattgaccagcataggtaagtggatcctgctgggttcatgggccatgagccagaccacatggagtgactgagggtcagccttcaggacaggaagatttcctcggggagccaacctgaatccccaccgtggagggatgtgttagtcctccaggagggtggcttcctagggaatctgaccccaggaggacacctttgctgaagccctgcaggggtgatggtggtccaggctggaaagttttccttcagtctcatgtggggcagctccctgctgacattccgtgcctgcgtctgaccttgaggactgcccgggccacatgagtggggcaacaagggctccagggtgccacggggattatgagtggggctctccatccccggagagaggtgggcaatcaggcccatgtggtcaggcttgtagggttcggtggtggggagagccaaggtttccctgggtccattaccgtcactgtttcacctggatcccatcacatgggtccatggagggagagcatgtgtgaacaggcaccccttgctccctttcccacatgagcccctggttggacatatgtcctcctcctgtccccccagatggtggccctggaggaggacttgtattggggccactggccccacgtcagtgttcgtcagcctggcatccagcccctggtgtgctgaagctcaggcccttcctgttgccctagtctcctgcactgagcttggtgagtccattcggggactgctggatgcatgggcgggtagggaggtgccctgggttgggtcgatgatgagaaccttatattgtcctgaagagaggtgatgacttaaaaatcattctcaaaaggattatgctgaggcccggcctaggttagaattttggaagaggattctcggatcctgagatccccagccaggccactgaatttttggctggagtccttgaggccctgaagggtgtctgagggctgctgaaacctgtctctgtgcttgtccgtgaggcaacccacattcgctgtgtggacttggtgttggccttcccgttctccggggatgggacatactgtcagcttgtccctgagtacccgttctcccctgtgtctttcagtgagctcttctgcccaggtggtccccctggtcttgatgggcataggtgagtacatcctgctggtgtcctggtccatgagccagggtgcgtacggttgccggaggtcacctttaggccatgtgggtttcctcagggagctgacctgaatccccactgggagggatggtttctccaggaggatgtcttccatagaaactggttccaagaaggatgccttccctgaagctgtgcaggggtgacgatggtccaggcaggaaagggtccattcaacccaatgcagtacagcttctagctgagactcggtgcacgggtgagggcttgaggactcccaggcaacatgaggggttccacaggatctccggaggccacaggtgttaagcaggaactctccatccttgaggcttgctgggggtggcgggatggcagcagggtgtgggtgatcgagtttatggattcaggccaagattcaagggtcaggccccacccattgccctgatgggaccaaccctgttgtgcaatgggctgtcccactggtgcctccttgagcctggtgtgggcagcgtggcttcaatggggttgggtcaaggctgagctctggcccgcacctggtggttggccctgggaattgtggactttgggccaagggtgcagcccgcctgcctctggaggtca

SEQ ID NO: 5 - DNA, 3284 bp, Homo sapiens
  source 1..3284: mol_type = other DNA
tgctctgcgtccatgttgggtcagtgttctgtaaggattccattacaagggtatcccctacccaccttccctacattgatacatgaggccctggttggacacatgtcctcctcctgtccctacagatggtgaccctgaagaaagacttcagttgggcctgaaggccccgggccagtgtccatcatccaggtgctcagctcccagtgcactgaagctcagacccttcctggttccctggtctcttgcactgagctctggtgagcacaactgggtgctgctggatgcatgcatggggaggggggtgccctgggttggggtggtgatgagaaccttgtattcttctgaagagaggtgatgacttaaaaaccatgctcaataggattacacttaggccgaacctaggtgagaatgttggaagaggacgttgggatcctattatccctggcagagccactgtattttgggctggagacctggaggccctgaaagggcatctggagggggcccaaccctgtctctgtgctcctgcatgagggtccccaggctccctgtccagccttggtgttggtgctctggtaccctggggatggaacaagttggtggctctttcctgagcccccattctccctttgtgtctttcagtgagcttttctgcccaggtgggcaccctggcattgattagcctccttggttgtccatgacctggctggtggcccgatcctgtgcccagtggtcccagggcagccctggcatcatacggggcagctgttttgggcagtggaggtgctctccaggcctgaagtatggcacagcctgggcattttacctgagaccagtgacctgcctgccttgtccttcctgggttcggtggcagggagggccaagggtgccctgcgtcagtgatgagtcagtattttgtgtggatctcatcacctgtccatggagggaaagcaagcatgagctggcacccaacacccccttctccgcatggagggtggtcacatgtcctccacctgtgcccccagatggtgagacttgaggaagacttccactgggcctgcttttcctgagccagtgcccatcacccaggtgctcagtccctggtgcactgaagctccagcccttcctggcgccctggtctcctgaactgagctgtggtgagcacatccgggtttcactggatgtttgtgcggggagggggttctctgggttgggtcaatgatgagaaccttatattgtcctgaagagaggtgatgacttaaaaatcatgctcaataggattacgctgaggcccagcctaggggagaattttggcagaggatgccgggatcccaagatttccgggagggccacttgtattttgggttggagacctggaggccctgaagggcgtctggaggtggcccagtgctttctttgcgctcctctgtgaggcagcccaggctccctttgtgggcttggtgtttgcgctcctcttctccagtgatggggcacgttgggggctcctccctgagtcctggttctccccttgtgtctttcagtgagctcttctgccaagcggttcccctggcattgaccaacataggtgagtggatcctgctggcatcatgggctatgagctaggccgcatgggtttgctgagggtcagacttcaggccccaagggtttcctcagggaaccgacctcaattcccaccatgtgactgccaggtttgcaacagtgcatagcttccgagctaccctccttggttggccgggagctggttggaggctgtggcccatgcctagtagtcacagggcaggcagactgccctgttttttcagggtgtggtgacggggagggccaagggtgccctgcatccatgatgggtcagggatttcaggagggatggtttctccaggagggcagcttccttgggaatcttacccaaggaggatgtcttccctgaagccctgcaggggtgacgatggtccagatagcaaaggtttccttcagccccatgcaggacatcacctagttgagatatggtatgtgggtcaggccttgaagactgcccaggccacctgaggggcccagaagggctctggggtgccacaggtgttatgtaggagctctcagtccccagacttgtttcgtggggttggtgacggggttgggttgtggaggttcaaggatttcatcccacctgtgaccctgaatggggccaaccctgttgagcaacaggccatcccaatggcacgtattgagcctggtggggtgtggtgcagtttcaatagggttgagtcaaggctgagatccatctggtgcttggtgctgctgctggccagccctgggacccgtgggctttgggctaagggggtagcccacatcctattgagggtaacccattctccgagtttgagtaacggttaacagtggcattctgaggtgggaaggcccactgtctagggtcctatactgggaatttgccaccccgggttcttctgagacaagtggtcacctccagggtcatggggtcttgggcccaaggaaaggagaagataccatgtggcccttgattggctacatgtggcccaggtctgtttgggaggtgtgtgggatgtggatggggtcccaccaatgaaagcacatggcctgggtccccaggctgtgattttcagtgcacacttggagagggtgcctcggcgggtacctaccacatccatgggcaggctatgtgactaccaggtttgcaacagtgcatagcttccaagctaccctccttggttggtcgggagctggttggaggctgtggcccatgcctagtggtcacagggcaggcagacggccctgttttttcagggtatggtgatggggagggccaagcatccatgatgagtcggtgttccacatggaacccattacgtgggtatctcttaccccttcctcatatggagatgtgaggccctatttggacacatatcctcctcctgtacccccagatggtgagcctggaggaagacttgcgttgggcccaaaggccctagaccagtgtctgtcagccaggttcccaacccccgatgcgctgaagctcaagcctttcctggcaccctggtctcctgcactgagctgtggtgagtatatcctggtcctgctggatgcatgtgcagggaggtgggtgccttgggttgggtcaatgatgagaaccttatattgttctgaagagaggtgattatttaaaaatcatgctcaataggattacgctgaggcccagcctagttgagaattttggaggaggacactgg

SEQ ID NO: 6 - DNA, 3079 bp, Homo sapiens
  source 1..3079: mol_type = other DNA
aggcccagcctaggtgataattttggaagaggacactgggtcccgaagccccctgcagggcccctgtgttttgggctggagaccttgaggccctgaaaggcatctggtggacccaactctatctctgccctcctcaatgaggcagcccagcctccctgtactggcttggtatccgggctccgggtctctggggacggggcacattggaggctccttcctgagcccccattctctcctgtgtctttcagtgagctcttcggcccaagtggtccccccggccttaagcggcataggtgagtgcatcctgttttgatcatgggccatgagtgttgcagcatggggtccccgacattcagccttcaggccactaggcttttctcaggctgcaaacctactctccaccagggagcgatggtttctcaggagggccacttccttcggaatctgacccatggagcacgcctttcctgaagccctgcagaggtgacagtggtccaggcaggaaggggttccttcagccccaatgtagggcagctcccagctgaaacttggtggacagatcaggcctggagtactgcccagggcatatggggaggagggacacagggaatgtggcgcaccctagaggtcacactggagttctgtctccctggagaggggtgagtgattgggcccacatggtcaggctgtggagattcaaggaatgctccacaccgatggccttgaatgggaccaaccctattgagcaatgggccatcttagtggcacctccttgaacctggtgggggatggcactctttcaatgaggttgggtcaaggctgagctctggctggcacccagtggtgctgcaggctggccctgggacttgtgggctttgggccaagggggaagcttccatcctgctgggaacaacccttccttgaggtccagtggcaggaaggggggcttcctgaggtggaaagccctactgttgagggacctgtcctgggaatttgctgtccaagctgcttctgagccatggggtcacctctagggtggtggggtcttgagcccaaggaaaggagaggacaccatgtggttcctgattggctgagtgtggcccggctctgtgtgtcggagttgtgcagggtatggattgggacccaccagtgaaagcacatggccctggaggtccgcaggcttggagagggcaccagatctggcacccaccatgcccaggtacaggctctgggactgccaggttgcagcagctcttggcatctgggccaccctccttggttggccaggagctagttggtggccctgtcccatgccccatgtccctggtatggccatggggtcatgcagaggaaccggccctggcatggaggtgctcttgcaacatgcagtagggtgcatcccaggcatcttgcctgagccaggtgacccgcttgccctgtctctccagggtttggtggcagggagggccaagggtgccccgtgtccatgttgggtcagtgttcagcatggatcccattgcatgagtatcacttagctccttcccagcattgatatgtgaggccctggttggacacttgtcctcctgtccctccagatggtgagcctggaggaagactcgtgttgggcccacaggccccaggccagtgtacgtcagccaggtgctcagcccatggtgtgctgaagctctgacccttcctgactccgtggtctcctgcactgagctgtgatgaccatatccaggtcctgctagatgcatgcgtggggaggcagggtgccctgggttgggtcaatgatgagaaccttgtattatcttgaagagaggtgatgacttaaaaatcatgctcaataggattacactgaggcccagactaggtgagaattttggaagaggatgctgggatcctgaggtccccaacagggccaccatattttgggctggagacctggaggtcctgaagggcatctgtagggggcccaaccctgtctctgcactcctccctgaggcagcccatgctccctattcgggatcggtattggtgctccacttccctgggggcaggacacgtgggtggctactccctgagcccccgttctccccttgtgtctttcagtgagctcttccaaacaggtgggctccctagcattgaccgacagaggtgagtggatccttctgggattatgggccatgagccaggccatgtggggtccccaaggttcagcctttggatcccgagagttgtctcagacagccaacctgatttgccacctgggagagatggcttctccaggagggtgggtttttgggaattggacccaaggaggacgccttccctgaagccctactgggatgatggtggtccaggcaggaaagggttccttcacccagctgagactcggtgttccagtaaggcctcaaggacctccctgccacatgaaggggcccagaggggctacgaggcaccacaggagtgacaggggagctcttggtccccagagtgtggtacatgaatgggcccatggggttgagtcgcagagattcaaggatgctgcaccacccatggccctgaatgtgactctcctcctggagcctggtggggggtggcgttttttccctggggttcggtcaaggtggaactctggtcagtacctggtggtaatgtaggtcagccctgggactcgtgggctttgggccaacggacagcccacatccctctgggggtcaccctttctctgaggtcgagtgtcgtgaagggtggcctcctgaggtgggaaagcccaatgtcgaaggacctgtactgagaatttgttgcttaggctgcttaggatccatgttgtcacctccagggtggtgggatcttgggaccaagcaaaggagagggcaccatgtggctgccagtggctatgtgtgactggctctgtgtctgagaggtgtgtggatggggacccaccagagaaagcaagtggccctgcagccccaagatgtgatttctggtgcaggcttggagagggtgcctatacctgctcccaccacgcccaggggcaggctttgccactgcaggtctgcagtgccacgtggca

SEQ ID NO: 7 - DNA, 2725 bp, Homo sapiens
  source 1..2725: mol_type = other DNA
ggttacccatcaggagggatggttcctccaggagggtcgcttccttgggaatctgactctaggagcatgcatttcctgaagctctgcagggtgatggtggtccaggcgggaaagggttccttcagcccaacataatgcagttcccagctgaaactcggtggcagatcaggctcaaggactgctcgggccacatgatgggggccacaggggatttggggtgccctgggggtcatgctggagttcttgccccaagaggggtgggtgatagggcccatgcgatcaggctgcggagattcaaggaatgctacccacccatggccctgaatgggactgaccctgttgaacaatgggccattttaatggtgcctccttgagcctggtaggtagtggcattgtttcaatgcggttgggtcaaggctgaactctggctggtgcccggtggtgcccacatgggctttgggccaagggggcagctcatatccctctggggtcaccccttccttgagattcagtggcaggaagggtgttctcctgaggtggaagacctgctgtcaagggacttgtcctgggaatttgccacctaggctgcttctgagccatggagtcacctccagggtggcagggtcttgagcccagggaaaggagacaccatatggtttctgactgggagtgtggcctggatctgtgtgtgggaggtgtgcagggcatggatggggacccatcagagaaagcatgtggccctgtaggcccccaggctttgatttctggtgtgtgcttgcagtgggcaccagggcagccctaccatgcccaggggcaggctctgtgactgccaggtctggagtggctcctggcatgaagaccacgctccttggttggccatgagcaggttggttgccctagcctgtgccctgtggtcctggtgcagccctggggttgtttggggagacagcaccggcagtggaggtgctctcaaggcctgcagtagggcgcagcccaggtgtcttgcccgagcccagtaacccacttgccctgtctttccagggtttggtggcagggagggccaactgtgccccgcatctgaaatggatcagtgttctgcatggatcccatcacaagggtatcccctacccaacttctgtgcattgttatgtgaggccctggttggacacatgtcctcctcctgtccctgcagatgctgaccctggaggaagacttgtgtcgggcccccaggccccaggccagtgtccatcagccaggtgctcagctcccagtgcgctgaagctcagacccttcctggcaccctggtgtccagcactgagctgtggtgagcccatccgggtcctgctggatgcatgaatggggaggggcctgcccagagttgggtcaatgatgagaaccttacattgtcctgaagagagatgatgacttaaaaatcatgctcaataggattacgctgaggcccagcctaggtgagaattttggaagaggacggctgggatcctgagatcccagtcagggtcactgtattttgggctggagacctggaggccctaaagggcatctggaagggggcccagccctgtctctgtgctcctccgtgaggcaacccacactccctgtgtgggcttggtgttggtgctctggttccccagggacagggcgggttggtggctccttcctgagcccctattctccctttgtgtctttcagtgagcttttccacccaggtgggccccctggcattgaccgacataggtgagtgcatcctactgggataatgggccactagatagtctgtgtggggttgctgaggtttagcctttgggccacaagggttttctcaggcagccaacctgaattcccacctgggagagatgggttctccaggaggcagcttttttgggaatcagacccaaggaggacaccttccctgaagccctgcaggggtgatggtggtccaggcaggaaagggttccttctgcccaactcgaggcccaatgcagggcagtgcccccctgagaattggtgctctggtaagggctcgaggactgcccaggccacatgaaggaggtcagaggggctatggggaaccatgggggcaacaggggagctcttggcccccggagtgtggtacatgaatgggcccatggggttgggtcacggagattcaaggattgcatctcacccacggccctgaatgcgaccatccctgttgagcaatgggccatcccagtgggtctcctggggcctggtgtggggtggcattgttttcctggggttgggtcaaggcggagctctgactggtacctgatggtgctgcaggtcagccctgggactcgtgggctttgggccaagaggcagcccgcatcctgctgggggtcgccttttctccgaggttgagtgtcgtgaaggatggcctcctgaggtgggaaggcccactgtcaaggcacctagactaggaattgttcctcaggctgcttgggagccatggtgtcacctccagggtggcgggatcttgggactaagcaaaggaggggacactacgtggctcccaattggccatgtgtggctggctctgtgcctgggaagtgtgtggagtatggatggggacccaccagagaaagcaagtggccctgcagtcccagaatgtaatttctggtgtaggcttggagagggtgcctatatgcatgcccaccatgcccaggagcaggctatgccacttcag

SEQ ID NO: 8 - DNA, 3034 bp, Homo sapiens
  source 1..3034: mol_type = other DNA
gttcaataggattatgtggagacttaacataagtgacaaacctaagacaagaagttagaaatccttgtttcttttaggtgaataattttggagcttgagaaaaagacaccattgtggcgaacttgctttgccaaaccctgtgtatgcatttttgggtaaacaacacattttcatcctttaagcattgtttccattatttataattcctaatagtatgagacatttaggactcattccttggttcttcttctctcattgtatctttcattgtatctttcagtgctgcaagagatgcaatcatcccattaatgaaagcagagtgactgccttcgtcacacatacattcaaaatagatcactaggctagtgatgattctctcttaaggctattatgtttactcatttaaaaaatctggatctcagtatagattttcttctcagtgagtgactgacctatctgatatgatgctatgacaaaatctttactttttacctgaatttggtgggattgaaagttgagaacatattaatttattttctgtttcacttgattaataatgcccaatctaacatgcagtgttcacttgggtacatatatacaactacatcaaattttttcttgttttaccttaaaagtgttaagatattgtaatggtatggccagtttactcactggtaaacttttcataacagaaatatcatgccagtggacacaaatatgtaacatcagtaatttgctttcctcatgaaacagagaactgaataagttgatgggtaaactagtatctgaattataaaccccaaatatcatgtgacttcatcattttcagtataatgaaaatacaggctgtgaatgcttccagttggtcctgaaagggcctttgaggtgagtaggttcaccaaagcggcaacatatgtgtcccagtgggcattctactcctcagtaagtgacttgacattctttatggcttcctcatgattaaaacaagtctttttattgaaattttactgaaataatgatgaaacaatgtaatattcttttcatctacatataagagtagacatagattaacctctaccttttttttttttttttttgagacggagtctcgctctgttgcccaggctagagtacagtggtgcgatctcagctcattgcaacctctgcctcccaggttcaagcgattctcctgcctcagcctcctgagtagctgggattacaggcacgtgctaccattcctggctaatttttttgtaattttagtagagacatggtttcaccatgttggtcaggctgttcttgaactcctgaccttgtgatctgcccacctctgcctcccaaagtgctgggattacaggcttgagcccccgtgcctggccttcacctctacctattataatattttcatgttattccattttgtcatgtttgtgggtcatttgatgagggttcacaatatgaaaactgggcattttggccaagatgcatgtaaatgaatacaactccatgtgccctgataactgatttctagatcctgagatgcaagtggcttccagagtgtctgtattttatgaaagttcatatacagtaaaccatgctaattgttactgatacagcttccttgaatatataaagcgatgattaatgggatgttttgtttctgcagaaaaccactgaattgcttctccaaagccaagtgacctgtatgatttgtccttattatattttgtgaaccagtggactaaaacctttataatgggtcatttttttagtattgtcaatattgctcaattaacaaacatgactcatatacacaggtgagatgtagttctccctttgacatttgctctacatctgtcccttagatgatgatatggaagaaaagcactctttggcctgttgtgactgggacagttgacagcacccaggtgtcctttaatgaaaatgctcttgacaccaatgcatcctagcatcacagcttcaggaagccttctcaagtgtgcatggggagtactatgtctttcatcaataatgaaatcttctgatttggtgagaaataatgccttaaaattacactcaataggattatgctgaggctcagcctacctagatgaaaatgctgaaacagataatagtgacttcttatttcccttcaaggggaattttattttgaacttgaaataacaaaatctttatgggtaacttgtttgcccaactgtatcagtgcacctataaatgaacaaaaaattctcagcctataagctttgcttcagtgacctttaatttctaatagtaaggtatattcaggtctcattctttggctttttctcttattgtgtatttcagtaagacatgctgccaagagatgtgccattctattataaaagatcagtagcttcctttaccgacgtgtatattctatctagaacattgagctatggaagactcccacctaagggaattagttttacaccttcaggtaatctgaacttcaatatgagttttgctttatgatataatccttatgacaaaatctttactttttatctgaacttcatacaatcaatattgaagattttttaaattcactgttacctagtccaatatttaatatacatttggctgtagatacatgactagacaaaaattttttatattattattgtgggggctctaacagtgtttcactgttttgagttactgatgaactttctgtaactgtgagaccatggtaatggaagagaccgtggtaatggggaggagaatgtgaggatcagtatgtactttgcctataaaacttaaatatttagtaatttgatcaccaaattgatactcaaatttcagttcctatcatgtgtaaaccaaaaagtatctgagatttagaagtttatttagaagtggattttgccaaggttaagcacatgctcagaagaaataaacccaaaatcacagaaaccgtctggggtctatgtctttctccaaagatgattttgagggcttcagtgtttaaaggggaaaagtgggctggaggggg

SEQ ID NO: 9 - DNA, 1809 bp, Homo sapiens
  source 1..1809: mol_type = other DNA
tgagaagagcaccctctggtgttgtcacagaggcaagtgctaccgcacaggcatgctgcagtgaatttaactgatcctctgtccctgcaaccgttgtttaaggatgctattctggtaagggtttacaagacaacgtaaatatatgtataaagagtatcttcaggagacgtaataatgtaaaaatcatgctcaatagaattaagctgaggctcaaaatagtgccaatccttgcaagaaggtgtgagaagtcattagatttggggggtatgggttattccattttgaacttggtaggaagaaacccttgggggaatcttacatcatttgcctctacctacttctaaaggaacttttaaaattcttttatcttgtctttattcttaatactcatacattattagagagaatatcttggtttattttttcctcatggtgtgcttcagaatgttcatccagcaagaggtgtgtcattacacggattatatatatgagttctgtctttcgtaattaaaaaatttcaagtggaccatttgaatattaaacattggccttcaaaatgattacatttacctgttcatacaaaattagtgagcaatgatctattctttccaatgagtgactgatctaatgaggtcctcattaaaactccttttttttttttttttttttttgcctgaacgtactttgatttaaagatgtgacatttagtaattcattgcaatcaactctttagtgattgcacaaatagacaatccttttctattattttattttttggttgctgtgtatggtgttatgatgtttgtggatcattgaggcactactcacaactgaaagactttggcaattttagggaatggcttgagattcagctataggttacccttgattgaaattgaatacaatacaggtagtctgaaatctcatttccttatcctgagtctcactcaggtgtcattagattcattataaagaaatgcatacactatgtatgatgccagttgttactgatatgaagacgataagaagcttttatttctgcagggaaataatcccttaccaggtcatcaaaagacatgatacctatgtgcttggcccttactgtacacggggaatcagtggtctaccacagcttaagtaacgggtcatatttggagtatcacacatctcagtcttgtagaaattaggaacagcaattaggagtcatgcacatataagagatgtaatcccaccctttgactatagcctactcttgtcttttacagaaaagactgtggaggaagaaaaccctttaccctgttgttcagggagaaactgacaccactcaactgcctggcactgaaaatgtggcatccagtccactttaccatcagtgtttaaggaaaccatctctggtaagcatatttgatccaagtgtgcatatgcacatgagtgccatgggctgggtcaatgatgagatgttaccttgaagagaaatgatgacgtaaaaattaagttcagttggattacgctgaggcccagcctaggtaacaattgtgagacaagaagtttggatttcttagtttccttccattggggactttattttgggattctggcaatggaatcttattgcgaaacttgttttgcacgcttctatctgtgtgcttctaagtaaactataaattctgattctttaagtgctatttaagtttcctacaattcctaattttcttggatatttggcactaagacccttgctctttttctctcattggatgatttagtaagtcctactggcactattctattgagtggaggtgagaggct

SEQ ID NO: 10 - DNA, 1941 bp, Homo sapiens
  source 1..1941: mol_type = other DNA
tgtttgcccatgaaacttgaaattagataaattgatgagcaatctggtgcctgacttttctttctcaagtacctttggcttcgttttcattatgatgaaagtgtgtgctgtgaataatgccaatggtaaataagacctttatggacacttatatgaatgacagtcagggccgttccttcaggccacatgattatcgttatgcattcttcttctcaatgagtgactagcatacttgccaggatctacaagagggaaactttattttttactaaaattgcatcgatataatgttgacaagttttgtgattattggtatcatctctaagatgtgatgctcatttggtctaaatatagaagttaacatattaacttctaatttatatatgtcaggttgctatgtttttacaatgtttgtggtttattgatgatgactcacaacataaagactatggcattatggctacatgttctaattctgatatatacatatcccatcattcattaaattaaatacaattatgagtagtatgatgtctgttttctaaaatctgagtttcacctggctccagtatgatcattatatgatggagtgcaagcaacatgttaatgccaattgttattgatatggcttccttgaatatgtaaacaagactgatcaattgaaggtcttatttctgcattaaactacctgagatattctccaaattcaagagacctgtatagttagtctttattctgtttgatgaataaatggcctatagcctatctaatggatcagtttttccagtactaatcatatctcttcattaagaaatagcatgcataagccatacatacaggaggtaatatttgtcatcttgatctactgtcttatcctcctccccttagataaggatgactgaggaagagtactctttggcttgttgacaccagcacagctgacacacccagatatctgtttggtctcctgtgaactttcaaccaggatttaaggatgccactctggtaggtattttcagacaaagtgaatatgtgattgtagagttccatggcttgtttcaacaataagaccttatgttgtcttgaggacatgagatgacatgaaaactagactggctggtccttatcttttctggggtattctagtttgagcttaagataaagaaatatatttgggatatagtttctctcacctgtttctattagcttctagaagaccacttatcaattctttcctcctgatttagtggaaaacattttggcagttcctcaaaaagttaaagataaaattaccatgtaacccaacaattctgctcctaagtacacactcaaaagaattgaatacaaatgttcaaatgaatacatacacatgaatgttcatagcagcactattcactggctggagttccagaccagcctggccaacgtggcaagaccctgtctctactaaaaatagaaaaattagctgggcatggttgctcacgcttgtagtctcagctactcaggaggctgaggcacaagaatcacttgaacccaggaggcagaggctgcagtgagctgagatcacaccattgcactccagcatgggtgacaagtgagattctgtctcaaaaaaaaaaaagtgaaggtatgatgacaatgtcacacaaaatagtgaataccaataaaaagatagaaattattaaaaagaaccaaatgaaaattctggagttgaaaagtacaataactaaaatttaaaaatttattagaaaggctcaatagtagatttgaactgggagaagaaacaagaagtaaacttgaagatagaacaatagagattatgttcaaaagaacagaaagcataaagaataaagaaacaaagagagtctcagagaatgtggaacatcattaagcataccagcatacacatatgggagtaccagacaggcaggggagatacaa

SEQ ID NO: 11 - DNA, 3240 bp, Rattus sp.
  source 1..3240: mol_type = other DNA
actactgtgctcttagagatccactgagaacagtcaaacaactctgaagcagagtaatgggacccaaaatgaaaactaatgacaatttttttaagagttaataatcaattatttgaaagattttaatttgactaacaagatttcataaggtacgtgtatatctggttattcaaaagctataattgagaattgagactggagtggagagcatttagctagtcacatgagagattatcttagagtctcatttccatcctccttacactctgctcaccatatggttccaaaaagaattcatattttgatgtagatccataaggcatagttaagaattccctaatcttgcacagtttgatgagtagaacacagtgtccaacaaaccaattactttttctcattttcctttaatatggattctttgtgtaactctcaaatgatagtagttccatagatcttgtccttagtcttctctcttcatttcatcactgtttcctgaaagtattctagggaaactgtaaggatgattgccatgctagggccactaattttcaccttcagcgaagaattctgtagacttaccttttattgatttgggacaagttcccttgaataatctgacactataaaaacaatgtgattcagcatactacaactcccctgcctaatctgtcccattgaaaacccaatagcaccaatatatccctccatcattcatatcaaatatgtgaaatctgtactcttgagccttctttatcccatacactatcccttctcttttgtcatgctgcatcttttgggaattatagcaacaatctcataaatacaaagaatacacaaatccagttcctatggtaatacaagtatgatttatgtctttgcctcttaagatatgacaaacaaaatctgaagtctacaaggcccaccatgatctctacagttccccaatcacattcagctgcctatcaaaacccaatactccacctgttaaaatgctcgtcttttctgcctggtaaccatctaattttttacagtttggggatctgaattctcagtctaaactgagcatttatacaatcctgctataaactccttgtgctttatatacatacatacatgtcccaatttgagcttctctgtaaaacaccttaattgaacaaaacagctctgaattgatctgaaacatctgcaacttgcaaagcagcaaacacacaatagtacttgaaaaagtgttaatggaaatttcaaactttccttaagacaaatgtaagttgtaaaatctttactacaggggctggggatttagctcagtggtagagcgcttacctaagaagcgcaaggccctgggttcggtccccagctctgaaaaaagaaccaaaaaaaaaaaaaaaaaatctttactacagaaatctttcatcatagaattatgtcacagatattattattccttatttcttaactcctttgcataggtctgctttgttgaattatgcagatttgaagtgagtgaagtactggtggggtactgaacatgtcttttattttaaacacagtctttaggtctagtgatgaaacctcaagctttgagatgtttctgttcatattcaggaaactgttttgtttttttgttttgtttttttttttttaagttttacaagtactaaacagcacaaatagtccatagcagcgaaacattaaaattaccaattcttaattgaagacaaaagtcttcctcctgctcaggccctcctatagcaactcaaaacaccctccctacctattttaacaggtacttagggaacttctacacttcaggaaatattgtcttactcatcgagttgttagaattatgacaagcagtagtgggaaatggtatattaatagctttatttcaagaatttaccttatgggcaaactcaacttgcttggctgttagtttccaatgcttccaataccatgaaagcatgtttaatggatgacactcagcttccaagtacatggtttactaaggaagaaagcagttgatggctagtttcttataatagtgatatatgtatggctcatgaaaaacacccacctgaacccaagactggtgttaacttcactttattcagctgtcaaatggcactttaggcatgtcttaccttggaatcctttcaagagtcaattaactctcccacatcatccagaatctaaagaccacagagtaactttcaaaatatataatttttaacctcactggatttaagaacagttggaaagctccaggtatattttgagactagtgtcagtattaccctgatagcaaccagacaaagataacaagaaaactacaggccagaaacttttatgaatataggtgcaaagatgctaaacaaaatactagcaaaccaaatccagcagcatagaaaatggtttatgtagcatagacaagtggggttattccaggaatgcaaggttcgttcaatacaaaaattccatcaatgtaatacaacatatcaataaaggacaaaaactacatgacaacctttttagatacagaagcatttgacaaaattcaatacccttttttgattaaaaaaaaaaaaagactcagtaaggtaggaatagatggaatatttctcaacttgcagtatctatgaaaatcccacagctaacaacatatttaatggtgaaaggcctaaagctttcccactacaatcagaaacaagaaaaagttgtacactcttgccacttctatttacttctatatactggatgtgctagccagggcacttaggcagggaaatcaaataaaaataattcagattggagaggaaacaaaagtatttatattcacagatgcacaatcttatataaaaaattttaaaaacaaaatcagttcagaatagctatggtatgtaaggttaatacacgaatatcaattctttctagatgtacaatgagtaatccaaacaaatattttttttaaaaaaaagttgttttataatagcatcaagtgaaaaacatttaacccaaaaaagccaagatgtatacactgaaatctataagaacaacattaaaggaaattacaaacctaaataaatggaaagacactccatgtttatggattagaaaatttaatactgttaaaataccaccatacttcaatctgatgtattgattcaacacaaatactatcaaaatcacagatgctttttatgtagaaattaaataaatcctaaaatccaagtggactgcaag

SEQ ID NO: 12 - DNA, 2000 bp, Rattus sp.
  source 1..2000: mol_type = other DNA
agaacatagagagagaatctgattagtttgggagtagtttataaatcttcaactgggctggactccacgtaatgtctttccaaaaatcaaacctttgaataaattttcaaaaaaaatctcactccacagggaaagtgtacatgtggagaaattgaagtttccggtgttgctttagattacacattcttcccaatttcattcaaagtttttcatcctgctgttactaacattcagtgcagaatctatcaaaaaacaaatccttccaaggaaatagtttcaagagcaaacctggcctgaaagaacctgataccactgtgaggcatgacaatatggcctgaacgagtctcagaaacattggtaaccttctcagtttagagatatggcaatccattaagcctaacacagatggatccctggctgaatattggtcagtcttttctgtacaactagtcttgaaagcattcacttaccagcattgggcactgagttttaccatgcacattggactttgcagaagaatgatcactcccttgcaggctgaatgagaaacaaagggtttgcttcaaacagtctgtaaatatctctgagtagtccttcattaattgtcctctctatgttacttgcgtaatgcaccctgagtgttcttcactgacaatgtttaggtaaagttcaaatgtgatctatgtaattgcttcaagagtgaggctgtgttgccatgcattggtcttttgtacatttattgacctttctcacaagagagggatttaaacatctggatgctgggaatggtagtatagaagaggcaactgcaagaggtattcttctcagtggcattataactttagagctcttatacagtctctcactttaaaagtattctatgtaaaaagggcaaacaagtgccttcacatcttgctctttcctcctgtacagaagacattcctcaaagactcaagacagggttcccatgcatcactagaatagtcttcaaccctgcatcccttccctcttgcaagtgaccattcgtaactctgagacagtgttggcagccaaggtaagccattgtgttaggtgatattaaattatgtataggtatgttctacctggatctggatcaatgatgacaacaaaaagtcatgcagagagatgatgataaaaaatcatgctcaataggattatgctgaggcccatccagggggacagggttacaagcagtccttgttctctttgagtacaatttatgggactcttctgaagtcctctgatgcccttgtgcagtttcccctcaccaggaatctggacactctcagtactcttttgttggcaaacattgcctgctaagtaccacctagtctcacattctacctgtatgtaagtctgtgaggcatatcaactgctcatgaccctgcactttatgaggtcttgcagtcatctggcatgcccttgtatcccagaaaatcacatatctaaggtgagtgaaaagatcatgtctaatgactccccctactacatctgatagagctaatatctaatatatacaaagaactcaagaagttagacaccataaaatcaaataaccctattaaaaatggaatatggagctaaacaaagaattctaaactgaagaatatcaaatatatgagaagtacttaaaaatgtccaatatccttagtcatcaggaaaacgcaaatcaaaaagccctgagattctacctcacaccagtgagaataactaagatgaaaagctcaggggacaacagatgctggtgaggatgtggagaaagagaaacactggtggtgggattgcaagctggaacaaccattctagaatgcagtctggaggttccccagaaaattcgacataggaatccctgaggatcctgctataccactcctgggcatatactgaaaagatggtacaacatataatgaagactatgctcacagtagccttatttattatagccagaagctgcaaagaacccagatgtcctgtaacag

SEQ ID NO: 13 - DNA, 2786 bp, Rattus sp.
  source 1..2786: mol_type = other DNA
aggtttatgcaagggttcccttgaatccatagaatagctttcaaccctgcagccctctgtacttactagtgaccattcacaattccgaggcagtgttggcagcccaggttaaccatggtgttaagtgatactaaactctgggtgtcctgcctgggtctgggtcaatgttgacaacaaaaagtcatggacagagatgaagttataacaatcatgctcaatggaataatgctgaggcccaaccagggatacagggttacaagaagtcctttttctttttgtcgaccacttgcaggactcttctgagaacttctgatgcctttgtgttgtttcccttacaaggaacctggacaccttcagttcatgtttgttgacaaacaccacctgataagtagcacctagtctcacattctacttgtgcaagtctctgcaaagcccacccaatgctcatgaccctgtctcttctcatgtcttgcagtcatttagaatgacccctgcagcccagagaatcatagatcttaggtaaatgaaaagatcttatctaatgggttcaccaactaatgacctatgcctccaattttgacctctgaatctcagcccatctaaagtttttttttaattaaaaacaaaataacagaacatggcaactaaaggacaattctcagtatgttcacccctgtagaactgcagattttcatgacatacagattttcctaactatcaataaattttacacatcaatttgagctacttggcataggtggaaggggaaaacacctttctgtacttgaatgccctttttgttgtttacaatttgaggagttgcaggacatcaatacaggattccaagcctgtgcctagttttaaattttgcatgacattggagagcctagcaccagagtctgaattagtaaatgagtagtacctataaatttccaactggcctggtctccacgtgtattcacaaaaagcaaagctttgagcaaattttcaaaacctcttaccacacagggaaacagtacatgtggagaacttgaagtttcagtagttgccttgggtaccattacctttcccacttccttcacagtgtttcatcctgctcttactaaacttcactatggaattcatcaggaaacaaatcctatcaaggagatggttccaggaacaggcctgttctgaaggaacctgatgtcactgtgagacatgaaaacatgggctgaaccatctcagaaactttggtcaccttctcagtatagtgatgtggcaatccattaaaccctaacacaaatgaatccatggccaaatattaatcaatctcttctctgccactgactctctgcagcattcacttaccagcattgggcactgagttttacgtacaggttggactttggaggggaatgatcactcccttgcagaaacaaaaggtaccctacaaacagtctctaaaatttctatgtagttgttcacttatcgtcttatccatattactcaggaaagtcaacttgagtgttggtccatgacaatatttaggcaaagaacagatgtgatctgggcaattgattcaagaggaagatgtgagcctgtgctgcaatgcattggccttttccactttcgttgacattaataacgagatgggaagaaacctggagaatgggcacggtagtatagaaaaggcagttgcaagagggattctgctcagaggcatcatacgtgtagagctcctatacagtctctcacattaaattattttattttatttatgttttttttaattttttattagctatatttctttgcttacatttcaactgttatttcccttcccggtttcctgtccataagcccccattcactcccttccccctcccccatatgggtattcccctatacatcccctttactgcccccccatattcccctgcactgggggtccaaccttagcaggaccaagggcttccccttccactggtgccccaacaaggctattatctgctacatatgcagttggagccctgggtaagtccatataaagtctttcggtagtgctttagtccctggaagctctggttggttggcattgttgtttttatggggttgcaagctccttcaaccctttccatacttcctttaattacccccaagggggtcctgttctcagtttagtggtttgctactagcattgacctctgtattagacatgctctagatgtgtctctttttcttttctttttttatctttattaacttgggtattccttatttacatttcgattgttattccctctcccggtttctgggccaacatcaccctagcccctccccctccccttctctgtgggtgttcccctccccatccttcccccattaccaccctctctccaacaatcccatttactgggggttcagtcttggcaggaccaacggcttccccttccactgctgctcttactaggctattctgaatgtgtctcttaggcagaggaatggattcagaaaatgtggtacatctacacagtggagtactactcagctatcaaaaacaatgacttcatgaaattcataggcaaatggaatgaactagaaaatatcctgattgctgtaactcaatcccagaaaaacacacttggtatgcactcattgataagtggatattagccaaaaagctcgaattacattattttattaaagaagggcaaacaagtgcctttaaatcttgatctttcctcctgtagagaagacattctccaaagattcaagcaaaggttcccttgcatccctacaagagc

SEQ ID NO: 14 - DNA, 2800 bp, Rattus sp.
  source 1..2800: mol_type = other DNA
tggcaaggggaacccaggaataagactttagtttagagatgaagaaattggcgataaattttctgcatcaaaagacactccaagaattattgccccaaagagagcttcgcaaaatttttcacataactctcaggccacagggaaactagccagtagggtagattgaagttgaaggtgtggctttggctacctatacccttctatgtgtcttcacacagcttcattcttgatcttactaaaatttacttcagtattcatcagaaaacaaatgctatcaaggagatggttcctcgagctcttgggacagaggaacgtgatgtcaatgggatacttgcaaacaccaactgatctagtcacacgaattttggtcacattctcagtttgatcacatggcaatcactgagtgctaacgctcagggattcatggccaaaatacttgttgcattcctctgggcaacagatactcttcagcattcacttaccacctgtgggaactgattgttaccatgcagtattgaaatggataggaatgaacataacctagcacataaaatctgaaatgcacaagtctcattaaaatttgattaaagtttacgtaactgttcgtgtattgtccttccaatattactcaggtaaattcacctttgccttggtttctgacaatgtttagcaattgaaagatttgatcggtgtatatgacaaagagcatgtgtcatccccttcatcaatgatttggttttgggtacctattgacctattgatcgcttagggaagtaactttccatgtgttgggaatgtcagcatggaagtggatgttgcaagagggattctactaagtggcatcatacattagacttcttaaacagcctcctaaaattaacatgattgtatgaactgaagggatacaattgccctcacatcttgctctttccttttagagaggacagtcttcaaattaaagctaggtctacccttattccacacaagagcgctgtgcccttgtaccctctgcacttgcaagtgaggattcaaattctgaagatttgttgggagcccaggtaagccatggtgcaggtgaaattacattattgacagttgtgtcctacctgtgtctgggtcaatgatgacaacagaaagtcaagaacagaatgatgacataaaaatcatgctcaataggattacgctgaggcccaaccagggagccggggtaacaacactacttagtctctttgaggaccacttgcgggactcatctgagctgctctgatgaccttgtgctcttttccctctgtgggaacctggacaatcttagtgcaactgtgctggcaaacactgcctgccaactagcacctaatctcattatctgcttgtgtgcaagtctgcaaccccacccgggtcatcattaccctctctgctctgatgtcttgtagtcacctggcatcaccccagcggtccagtggatcactactgccaggtgtgtgaaatgatcacgattaatggatttaactattcatgagatattcctcagtattttgaccaataatttcagcccatcaaaggtttcttaattaaaaataaagcaataccagtcagaaatgaattgcacttctaagtatgttctgtccctttgaactgcactatttaaaatgtagagaaattcctttctacctgtagttcatacagccataaaattgaatttcttggaagagatcctagatgatactatctacacaaatggagggatctggaatatgaatataccttttgaatttggagtaatttcagtccacacacaggatcacaaatccaagctggtgcacagagatcgatttccaatggcaaggggatcccaggaataagactttagtttagagatgaagaaattggcgataaattttctgcatcaaaagacactccaagaattattgccccaaagagagctttgcgaaatttttcacgtaactctcaggccacagggaacatagccagtagggtagattgaagttgtaggtgtggctttggctaccaattcccttcgatgtgtcatcacactgcttaattcttgatcttactaaagtttacttcagtattcatcagaaaacaaatgctatcaatgagagagtttcaggagctcttgggacagaagaacatgatgtcaatggcatacttgcaaacaccaactgatctagtcacacgaattttggtcacattctgagtttcatcacatggcaatcactgagtgctaacgctcagggattcatggccaaaatacttgttgctttcttctgggcaactgattctcttcagcattcacttaccagctgtgggaactgatttttaccatgcagtatggaaatggtgtggaatgaacacagactagcacataaaatttgaaacgcacacgtctcttgaaaatttgattaaagtatatgtaactgttcgtgtattgtactccccatattactcagagaaattcacatttgtcttggtttctgacaatgtttatcaattgaaagatttgatcggtgtatatgacaaagagcatgtgtcatccccttcatcaatgatttggttttgggtacctattgacctattgatcgcttagagaagtaactttccatgtgttgggaatgtcagcatggaagaggatgttgcaagcgtgattctactaagtggcatcatacattagacttcttaaacagcctccttaaaattaatatgattgtatgaactgagggatacaattgccctcacatcttgctctttcctttttagagaggacagtcttcaaattcaagct

SEQ ID NO: 15 - RNA, 20 nt, synthetic construct
  source 1..20: mol_type = other RNA
  ncRNA 1..20: ncRNA_class = guide_RNA
gatccatttgtgttaagctg

SEQ ID NO: 16 - RNA, 28 nt, synthetic construct
  source 1..28: mol_type = other RNA
  ncRNA 1..28: ncRNA_class = guide_RNA
gatccatttgtgttaagctgtaatgggt

SEQ ID NO: 17 - RNA, 30 nt, synthetic construct
  source 1..30: mol_type = other RNA
  ncRNA 1..30: ncRNA_class = guide_RNA
cttccatgacgcagaagttaacactttcgg

(19) United States
(12) Patent Application Publication
(10) Pub. No.: US 2026/0256950 A1
(43) Pub. Date: Sep. 3, 2026

(54) COMPOSITIONS AND METHODS FOR TREATMENT OF ANGELMAN SYNDROME

(71) Applicant: The Regents of the University of California, Oakland, CA (US)

(72) Inventors: David J. SEGAL, Davis, CA (US); Ulrika BEITNERE, Davis, CA (US); Henriette O'GEEN, Davis, CA (US)

(73) Assignee: The Regents of the University of California, Oakland, CA (US)

(21) Appl. No.: 18/843,044

(22) PCT Filed: Mar. 2, 2023
(86) PCT No.: PCT/US2023/014377

§ 371 Date: Aug. 30, 2024

Related U.S. Application Data
(60) Provisional application No. 63/316,160, filed on Mar. 3, 2022.

(51) Int. Cl. A61K 48/00; A61K 9/00; A61K 38/45; A61P 25/28; C12N 9/22; C12N 15/11; C12N 15/86

(52) U.S. Cl. CPC A61K 48/0058; A61K 9/0019; A61K 9/0043; A61K 38/45; A61K 48/0075; A61P 25/28; C12N 9/226; C12N 15/111; C12N 15/86; C12Y 203/02; C12N 2310/20; C12N 2750/14143; C12N 2750/14145; C12N 2800/22

(57) ABSTRACT

Compositions of a Cas13 protein and a guide RNA that targets a portion of a Ube3a-ATS transcript are provided herein, along with vectors that can continuously produce such compositions in cells, and cells that contain such vectors. These compositions, vectors, and cells can be used in methods for cleaving a portion of a Ube3a-ATS antisense RNA transcript and unsilencing the paternal UBE3A gene, as well as methods for treating Angelman Syndrome without impairing expression of Snord116 and/or Snord115.

### CROSS-REFERENCE TO RELATED PATENT APPLICATIONS

This application claims the benefit of priority to U.S. Provisional Patent Application No. 63/316,160 filed Mar. 3, 2022, the disclosure of which is hereby incorporated by reference in its entirety for all purposes.

### BACKGROUND

Angelman Syndrome (AS) is caused by disruption of the UBE3A gene in the brain in the copy of chromosome 15 that came from a subject's mother (maternal copy). While there is an intact UBE3A gene from the father (paternal copy), it is silenced by Ube3a-ATS, an antisense RNA transcript. Activation of expression of the paternal allele of UBE3A has great promise to treat the underlying causes of this disorder and bring meaningful improvements to children affected by this syndrome.

In 2011, it was demonstrated that UBE3A gene therapy using adeno-associated virus (AAV) to introduce a UBE3A transgene into the brain could rescue some AS phenotypes in a mouse model of AS (Daily et al., PloS one 6: e27221 (2011)). Treated mice exhibited significant improvements in associative learning as assayed by fear conditioning as well as by the Morris water maze learning task. However, there are several limitations to this approach. For example, there are at least three major splice isoforms of UBE3A, some of which are nuclear and others cytoplasmic, suggesting that having all isoforms represented may be important. However, only one splice isoform can be packaged as a transgene given the capacity of commonly used vectors. Also, overexpression of UBE3A has been linked to autism. It may not be easy to ensure that UBE3A will be expressed at the appropriate level in all cells, and once in the brain the levels cannot be modified. Since this seminal work a decade ago, no AAV-UBE3A gene therapy has advanced to clinical trial. Therefore, there is a long-felt need to develop new therapeutic agents that will activate isoforms of the paternal UBE3A allele at the appropriate level in vivo.

An alternative approach to restore expression of a “healthy” copy of the UBE3A gene is to block expression of the Ube3a-ATS transcript. Along these lines, Bailus et al. (Mol. Ther., 24:548-55 (2016)) used a zinc-finger-based artificial transcription factor that targets the Ube3a-ATS promoter. This resulted in widespread UBE3A expression in an AS mouse model brain using its normal physiological promoter. However, silencing of the entire Ube3a-ATS antisense transcript raised new concerns about the loss of the Snord116 and Snord115 clusters of RNAs, which are spliced from the large Ube3a-ATS transcript. In humans, loss of SNORD116 expression causes Prader-Willi syndrome, which presents as a failure to thrive at birth but in adolescence changes to a hyperphagic disorder in which food satiation does not occur and affected individuals become highly obese (Angulo et al., J. Endocrinol. Ivest., 38:1249-63 (2015)). Therefore, there is a recognized need to develop new therapeutic agents that will unsilence expression of paternal UBE3A in AS patients without creating the negative side effects of impacting SNORD gene expression.

Another way to restore expression of the UBE3A gene is to inject chemically modified ASOs (antisense oligonucleotides) that use an RNAse H-mediated mechanism and are designed to cut off the end of the Ube3a-ATS antisense transcript that is complementary to UBE3A, in order to unsilence paternal UBE3A without affecting SNORD gene expression. In 2019, GeneTx and Ultragenyx initiated a Phase 1/2 clinical trial of a human targeted ASO “GTX-102” (NCT04259281). Patients received three intrathecal injections in a dose-escalation scheme over six months. Early data in five patients showed improvements in multiple symptom domains including sleep, behavior, communication, and gross and fine motor skills, based on the Clinical Global Impression of Improvement Scale for Angelman Syndrome (CGI-AS). Caregivers of the patients also reported improvements such as patients being more communicative, social, and responsive as well as showing improvements in gait and posture. The ASOs showed minimal toxicity at low doses, but at high doses caused serious but reversible muscle weakness in the legs and feet. Three ASO treatments for AS entered clinical trials in 2020. However, there are limitations to the use of ASO drugs for neurological disorders. These drugs are transient and must be reinjected periodically, so the preferred route of administration is intrathecal (into the spinal column). Angelman Syndrome children require full anesthesia for an intrathecal injection, which carries significant risks. ASO drugs are only effective for a few months and then require lifelong reinjection 3-4 times a year, causing recurring health risks and significant costs. Therefore, there is a need for AS therapies that do not need frequent periodic reinjection, particularly where the mode of injection carries significant risks.

An analogous drug used to treat pediatric motor neuron disease spinal muscular atrophy (SMA) is the ASO drug SPRINRAZA (nusinersen), which was FDA-approved in 2015. SPINRAZA was also hailed as the first ASO to treat a disorder in the central nervous system (CNS), and was the model for intrathecal injection of GTX-102. However, as a transient treatment, SPINRAZA requires six loading doses in the first year, and three doses every year thereafter. It is not clear yet if intrathecal ASO treatments would allow the drug to reach all the parts of the brain that need it, and it is unlikely that more targeted but invasive routes would be used because of the need for repeated injections. Also, SPINRAZA treatment incurs high costs, and currently costs $750,000 for the first dose and $375,000 every year thereafter. In comparison, a single-dose gene therapy for SMA called Zogensma (onasemnogene abeparvovec) that was approved in 2019 costs about $2.125 million, ultimately less than the cost of perpetual ASO therapy. Although exact costs of ASO and gene therapy for AS are not known, it is clearly imperative to develop a single-dose gene therapy for AS to reduce both the cost of treatment and the health risks of multiple anesthetic procedures each year. Thus, there is a recognized need for new therapeutic agents that are more permanent and lower-cost than a lifetime of ASO injections.

Accordingly, there is a need for a more affordable and less risky therapy for restoring endogenous expression of UBE3A in those affected by AS without negatively impacting SNORD gene expression. The present disclosure satisfies this need, and provides related advantages as well.

### BRIEF SUMMARY

The present disclosure provides a composition comprising a Cas13 protein and a guide RNA that targets a Ube3a-ATS transcript. In some embodiments, the guide RNA targets a Ube3a-ATS transcript downstream of the coding region of a Snord115 transcript. In some embodiments, the guide RNA targets a Ube3a-ATS transcript downstream of the coding region of a Snord116 transcript. In some embodiments, the guide RNA targets a Ube3a-ATS transcript upstream of a sequence complementary to a polynucleotide in a gene encoding a protein isoform of UBE3A. In some embodiments, the protein isoform of UBE3A comprises SEQ ID NO: 1, SEQ ID NO: 2, or SEQ ID NO: 3.

In some instances, the guide RNA targets a sequence complementary to a polynucleotide sequence in a UBE3A/SNORD region. In some embodiments, the polynucleotide sequence in a UBE3A/SNORD region is a sequence within SEQ ID NO: 4, SEQ ID NO: 5, SEQ ID NO: 6, SEQ ID NO: 7, SEQ ID NO: 8, SEQ ID NO: 9, or SEQ ID NO: 10. In some embodiments, the polynucleotide sequence in a UBE3A/SNORD region is a sequence within SEQ ID NO: 4, SEQ ID NO: 5, SEQ ID NO: 6, or SEQ ID NO: 7. In some embodiments, the polynucleotide sequence in a UBE3A/SNORD region is a sequence within SEQ ID NO: 11, SEQ ID NO: 12, SEQ ID NO: 13, or SEQ ID NO: 14. In some aspects, the polynucleotide sequence in a UBE3A/SNORD region is about 21 to about 32 nucleotides long.

In some embodiments, the Cas13 protein comprises a Cas13a protein, a Cas13b protein, a Cas13c protein, a Cas13d protein, a Cas13X protein, a Cas13Y protein or a variant thereof. In some embodiments, the Cas13 protein comprises a Cas13b protein or variant thereof. In some embodiments, the Cas13 protein comprises a Cas13d protein or variant thereof. In some embodiments, the Cas13 protein comprises a Cas13X protein or variant thereof. In some aspects, the composition further comprises a pharmaceutically acceptable carrier.

The present disclosure also provides a vector that encodes a polynucleotide sequence encoding a Cas13 protein and a polynucleotide sequence encoding a guide RNA that targets a Ube3a-ATS transcript. In some aspects, the guide RNA targets the Ube3a-ATS transcript downstream of the coding region of a Snord115 transcript. In some embodiments, the guide RNA targets the Ube3a-ATS transcript downstream of the coding region of a Snord116 transcript.

In some embodiments, the guide RNA targets the Ube3a-ATS transcript upstream of a sequence complementary to the coding region of a protein isoform of UBE3A. In some embodiments, the protein isoform of UBE3A comprises SEQ ID NO: 1, SEQ ID NO: 2, or SEQ ID NO: 3. In some instances, the the guide RNA targets a sequence complementary to a polynucleotide sequence in a UBE3A/SNORD region. In some embodiments, the polynucleotide sequence in a UBE3A/SNORD region is a sequence within SEQ ID NO: 4, SEQ ID NO: 5, SEQ ID NO: 6, SEQ ID NO: 7, SEQ ID NO: 8, SEQ ID NO: 9, or SEQ ID NO: 10. In some embodiments, the polynucleotide sequence in a UBE3A/SNORD region is a sequence within SEQ ID NO: 4, SEQ ID NO: 5, SEQ ID NO: 6, or SEQ ID NO: 7. In some embodiments, the polynucleotide sequence in a UBE3A/SNORD region is a sequence within SEQ ID NO: 11, SEQ ID NO: 12, SEQ ID NO: 13, or SEQ ID NO: 14. In some aspects, the polynucleotide sequence in a UBE3A/SNORD region is about 21 to about 32 nucleotides long.

In some aspects, the vector comprises a polynucleotide sequence encoding a Cas13 protein, wherein the Cas13 protein comprises a Cas13a protein, a Cas13b protein, a Cas13c protein, a Cas13d protein, a Cas13X protein, a Cas13Y protein or a variant thereof. In some embodiments, the Cas13 protein comprises a Cas13b protein or variant thereof. In some embodiments, the Cas13 protein comprises a Cas13d protein or variant thereof. In some embodiments, the Cas13 protein comprises a Cas 13X protein or variant thereof. In some embodiments, the polynucleotide encoding the Cas13 protein is codon optimized for expression in a human cell. In some embodiments, the polynucleotide encoding the Cas13 protein is codon optimized for expression in a rat cell. In some embodiments, the polynucleotide encoding the guide RNA comprises a U6::crRNA cassette.

In some aspects, the vector comprises a promoter element that directs transgene expression to the brain and/or spinal cord. In some embodiments, the promoter element is neuron-specific. In some embodiments, the promoter element allows for continuous expression in at least part of the brain and/or spinal cord. In some embodiments, the promoter comprises the promoter from the human SYN1 gene (hSYN1).

In some aspects, the vector is an AAV vector. In some aspects, the AAV vector can cross the blood-brain barrier. In some aspects, the AAV vector encodes an AAV capsid that allows for enhanced transduction to cells in the central nervous system (CNS). In some aspects the AAV vector is AAV9.

The present disclosure also provides for a cell containing any composition described herein. The present disclosure also provides for a cell containing any vector described herein. In some embodiments, the cell is a neuron. In some embodiments, the cell is a stem cell.

The present disclosure also provides for a method for de-repression of UBE3A expression in a cell, comprising contacting a cell with any composition described herein. The present disclosure also provides for a method for de-repression of UBE3A expression in a cell, comprising transfecting or transducing the cell with a vector described herein.

The present disclosure also provides for a method for cleaving a Ube3a-ATS transcript in a cell, comprising contacting the cell with any composition described herein. The present disclosure also provides for a method for cleaving a Ube3a-ATS transcript in a cell, comprising transfecting or transducing the cell with a vector described herein. In certain embodiments, the cell is a neuron. In certain embodiments, the cell is a stem cell.

The present disclosure also provides for a method of treating Angelman Syndrome in a subject in need thereof, comprising administering to the subject a therapeutically effective amount of a composition described herein. The present disclosure also provides for a method of treating Angelman Syndrome in a subject in need thereof, comprising administering to the subject a therapeutically effective amount of a vector described herein. The present disclosure also provides for a method of treating Angelman Syndrome in a subject in need thereof, comprising administering to the subject a cell described herein. In certain embodiments, the therapeutically effective amount is an amount sufficient to de-repress the expression of all three major protein isoforms of UBE3A. In certain embodiments, the route of administration is at least one of intraparenchymal, intranasal, intracerebroventricular, intracisternal, intrathecal, intravenous, intramuscular, via the spinal cord, via the eye, or via the cochlea. In some embodiments, the route of administration of the AAV is at least one of intrathecal, intracerebroventricular, or via the intracisterna magna. In certain embodiments, expression of Cas13 and guide RNA is maintained for at least 16 weeks in the subject. In certain embodiments, the subject has a mutation that affects expression or activity of the maternal copy of the UBE3A gene.

Other objects, features, and advantages of the present disclosure will be apparent to one of skill in the art from the following detailed description and figures.

### BRIEF DESCRIPTION OF DRAWINGS

FIGS. 1A and 1B illustrate the overall approach to using AAV-Cas13 to alter UBE3A expression. FIG. 1A shows the UBE3A locus in mice. In AS, expression or activity of the maternal UBE3A allele is deleted or reduced. The paternal allele (shown) encodes a wild-type UBE3A (red), but it is silenced by the Ube3a-ATS antisense transcript (wiggle line).

FIG. 1B shows how Cas13 (scissors) are used to target the 3′ end of the Ube3a-ATS antisense transcript.

FIGS. 2A and 2B illustrate the effects of AAV-S1K-PHP.eB administered intravenously. FIG. 2A shows RT-qPCR data of Ube3a-YFP expression using 10¹³ viral genome particles/animal. FIG. 2B shows Western blot results of Ube3a-YFP and mCherry (AAV vector expression) using 10¹² viral genome particles/animal, *p<0.05.

FIG. 3 illustrates RT-qPCR data on Ube3a-ATS RNA following treatment with AAV-S1K-PHP.eB, *p<0.05, **p<0.01, ***p<0.0005.

FIGS. 4A and 4B illustrate the effects of intranasal injection of AAV-S1K-PHP.eB using 10¹¹ viral genome particles/animal. FIG. 4A shows Western quantitation of Ube3a-YFP expression. FIG. 4B shows Western blot data of Ube3a-YFP and mCherry (AAV vector) expression. *p<0.05.

FIGS. 5A and 5B show that AAV-PHP.eB-Cas13b designed to target to the 3′ end of the UBE3A antisense transcript, when injected intranasally at 10¹¹ viral genome particles/animal, can increase Ube3a-YFP protein expression. FIG. 5A shows quantitation of Ube3a-YFP expression levels, and FIG. 5B shows the corresponding Western blot.

FIGS. 6A-6E shows RT-qPCR data from hippocampi three weeks after intravenous injection of 1×10¹⁴ viral genome particles/animal of AAV-PHP.eB-Cas13d. FIG. 6A shows Cas13d expression in the samples. FIGS. 6B and 6C show that expression of Snord116 and Snord115 was unaffected, whereas FIG. 6D shows that Ube3a-ATS antisense mRNA expression was reduced, and FIG. 6E shows that UBE3A gene expression was correspondingly increased.

### DETAILED DESCRIPTION

### I. Introduction

Angelman Syndrome (AS) is a rare neurological disorder affecting 1 in 15,000 children for which there is currently no treatment (Angelman, Dev. Med. Child Neurol. 7:681-88 (1965)). It is a genetic disorder that manifests as delayed development, including problems with crawling, speech, balance, and intellectual abilities, and sometimes seizures. A person with AS is likely to need support throughout his or her life.

In AS patients, the maternal copy of the UBE3A gene is disrupted and the paternal UBE3A gene is silenced by an antisense RNA. Thus far, attempts to develop therapies based on heterologous expression of UBE3A or increasing endogenous expression of paternal UBE3A by reducing expression of the antisense RNA have been hampered by major drawbacks. These drawbacks include the risk of overexpressing UBE3A (which is linked to autism), exorbitant cost, the need for repeated injections of therapeutic material, and deleterious effects caused by disruption of the expression of other important transcripts, including the Snord115 and Snord116 clusters of RNAs, which are spliced from the large Ube3a-ATS antisense transcript.

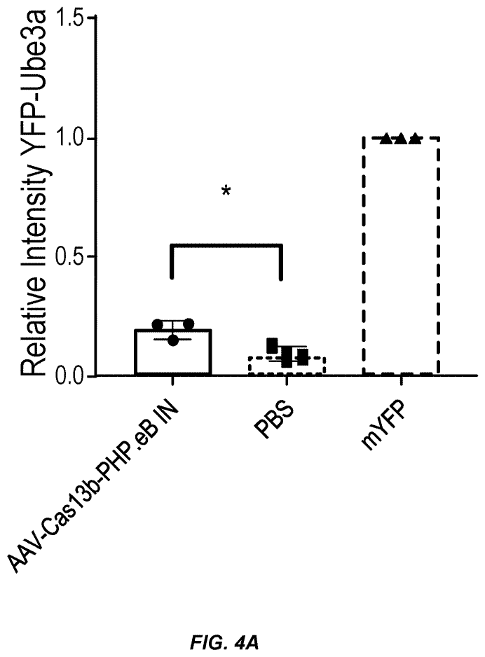

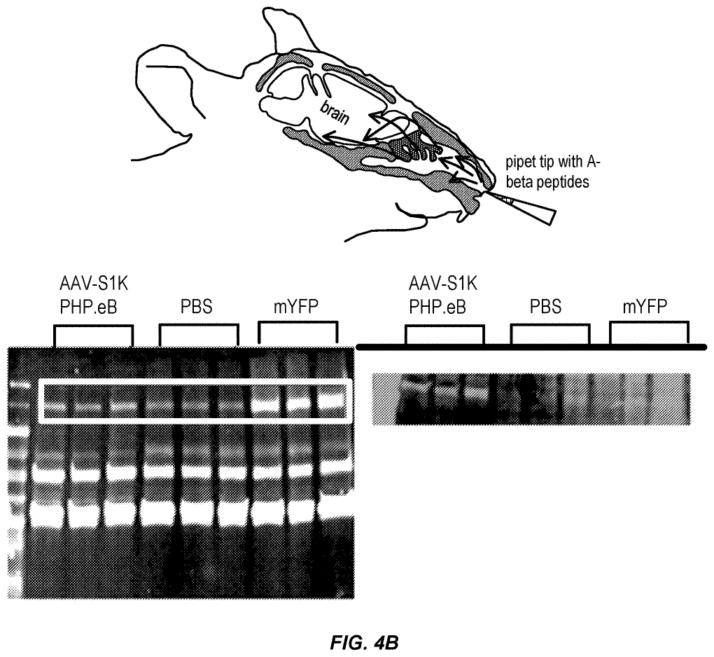

In certain aspects, the present disclosure provides compositions and methods for treating AS using an isoform of Cas13 that incorporates a guide RNA that targets a portion of the Ube3a-ATS antisense transcript and is encoded on an adeno-associated virus (AAV) vector. Targeting a portion of the transcript near its 3′ end rather than the entire antisense transcript allows for unsilencing of the UBE3A gene without significantly affecting Snord115 and Snord116 expression (see, e.g., FIGS. 4-6).

Accordingly, the compositions and vectors of the present disclosure are able to affect silencing by the Ube3a-ATS transcript of the paternal UBE3A gene in AS subjects without the risk of developing autism due to unregulated expression of UBE3A, without incurring deleterious effects on SNORD gene expression, and without requiring high cost or frequent injections.

### II. Definitions

As used herein, the following terms have the meanings ascribed to them unless specified otherwise.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

The terms “about” and “approximately” as used herein shall generally mean an acceptable degree of error for the quantity measured given the nature or precision of the measurements. Typically, exemplary degrees of error are within 20 percent (%), preferably within 10%, and more preferably within 5% of a given value or range of values. Any reference to “about X” specifically indicates at least the values X, 0.8X, 0.81X, 0.82X, 0.83X, 0.84X, 0.85X, 0.86X, 0.87X, 0.88X, 0.89X, 0.9X, 0.91X, 0.92X, 0.93X, 0.94X, 0.95X, 0.96X, 0.97X, 0.98X, 0.99X, 1.01X, 1.02X, 1.03X, 1.04X, 1.05X, 1.06X, 1.07X, 1.08X, 1.09X, 1.1X, 1.11X, 1.12X, 1.13X, 1.14X, 1.15X, 1.16X, 1.17X, 1.18X, 1.19X, and 1.2X. Thus, “about X” is intended to teach and provide written description support for a claim limitation of, e.g., “0.98X.”

The terms “peptide,” “polypeptide,” and “protein” are used interchangeably herein to refer to a polymer of amino acid residues linked by covalent peptide bonds. All three terms apply to naturally occurring amino acid polymers and non-natural amino acid polymers, as well as to amino acid polymers in which one (or more) amino acid residue is an artificial chemical mimetic of a corresponding naturally occurring amino acid. Unless otherwise specified, the terms encompass amino acid chains of any length, including full-length proteins.

The terms “nucleic acid,” “nucleotide,” and “polynucleotide” refer to deoxyribonucleic acids (DNA) or ribonucleic acids (RNA) and polymers. The term includes, but is not limited to, single-, double-, or multi-stranded DNA or RNA, genomic DNA, cDNA, and DNA-RNA hybrids, as well as other polymers comprising purine and/or pyrimidine bases or other natural, chemically modified, biochemically modified, non-natural, synthetic, or derivatized nucleotide bases. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), orthologs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

When a second DNA or RNA sequence is “upstream” or “downstream” of a first DNA or RNA sequence, it is located outside the first DNA or RNA sequence on the same polynucleotide strand. When it is “upstream” it is closer to the 5′ end than the 3′ end of the first DNA or RNA sequence, whereas when it is “downstream” it is closer to the 3′ end than the 5′ end of the first DNA or RNA sequence.

“Protein isoforms” are highly similar proteins that originate from a single gene or gene family, potentially derived from alternative splicing from the same transcript.

“Ube3a-ATS” is an RNA that is transcribed on human chromosome 15 in the region comprising the UBE3A gene locus. It encodes an antisense transcript with respect to UBE3A transcripts, and is expressed mainly from the paternal allele in the central nervous system. Ube3a-ATS is part of a larger antisense transcript called LNCAT (large non-coding antisense transcript). LNCAT is alternatively spliced and different splice variants vary in cell location, tissue distribution, and expression during different stages of developmental. Ube3a-ATS also encodes Snord116, a non-coding RNA that modifies other small nuclear RNAs, and Snord115, a small nucleolar RNA involved in the alternative splicing of several mRNAs, including the serotonin receptor 2C pre-mRNA. Loss of Snord116 expression causes Prader-Willi syndrome.

As used herein, a “UBE3A gene” encodes an E3 ubiquitin ligase. Human UBE3A is expressed in most tissues, but is only expressed from the maternal allele in almost all neurons (Mabb et al., Angelman Syndrome: insights into genomic imprinting and neurodevelopmental phenotypes, Trends Neuroscience 34:293-303 (2011). Human UBE3A has three known protein isoforms from different transcripts, likely with different locations and neuronal functions (Sirios et al., Abundance and localization of human UBE3A protein isoforms, Human Molecular Genetics 29; 3021-31 (2020)).

“Ube3a-ATS,” “UBE3A,” “Snord115” or “Snord116” in the context of studies with non-human animals (e.g., mice) refer to the corresponding ortholog of human Ube3a-ATS, human UBE3A, human Snord115, or human Snord116, respectively, unless otherwise specified.

“UBE3A/SNORD region” is a region on a chromosome that contains the UBE3A gene and the intervening chromosomal DNA up to the coding region coding for Snord116. For example, the human UBE3A/SNORD region is on chromosome 15 at 15q11-q13 and comprises the human UBE3A gene up to the human SNORD116 gene, a region greater than 40,000 bp in length. The orthologous mouse genes are located on mouse chromosome 7, and the orthologous rat genes are located on chromosome 1.

The term “pharmaceutical acceptable carrier” refers to a substance that aids the administration of an agent (e.g., Cas nuclease, guide RNA, transfected cell, etc.) to a cell, an organism, or a subject. “Pharmaceutically acceptable carrier” refers to a carrier or excipient that can be included in a composition or formulation and that causes no significant adverse toxicological effect on the patient. Non-limiting examples of pharmaceutically acceptable carrier include water, NaCl, normal saline solutions, lactated Ringer's, normal sucrose, normal glucose, binders, fillers, disintegrants, lubricants, coatings, sweeteners, flavors and colors, and the like. One of skill in the art will recognize that other pharmaceutical carriers are useful in the present invention.

As used herein, the terms “treat,” “treatment,” and “treating” refer to any indicia of success in the treatment or amelioration of an injury, pathology, condition, or symptom (e.g., Angelman Syndrome), including any objective or subjective parameter such as abatement; remission; diminishing of symptoms or making the symptom, injury, pathology or condition more tolerable to the patient; reduction in the rate of symptom progression; decreasing the frequency or duration of the symptom or condition; or, in some situations, preventing the onset of the symptom. The treatment or amelioration of symptoms can be based on any objective or subjective parameter, including, e.g., the result of a physical examination.

As used herein, the term “subject” refers to animals such as mammals, including, but not limited to, primates (e.g., humans), cows, sheep, goats, horses, dogs, cats, rabbits, rats, mice and the like.

The term “administering or “administration” refers to the process by which agents, compositions, dosage forms and/or combinations disclosed herein are delivered to a subject for treatment or prophylactic purposes. Compositions, dosage forms and/or combinations disclosed herein are administered in accordance with good medical practices taking into account the subject's clinical condition, the site and method of administration, dosage, subject age, sex, body weight, and other factors known to the physician. For example, the terms “administering” or “administration” include providing, giving, dosing and/or prescribing agents, compositions, dosage forms and/or combinations disclosed herein by a clinician or other clinical professional.

### III. Description of Embodiments

In one aspect, the present disclosure provides a composition comprising a Cas13 protein and a guide RNA that targets a Ube3a-ATS transcript, wherein the Ube3a-ATS transcript is an antisense transcript expressed near the UBE3A gene. The human isoform of this antisense transcript silences the paternal UBE3A gene in Angelman Syndrome patients. The guide RNA is designed to target the Ube3a-ATS transcript such that UBE3A is unsilenced, and so the symptoms of Angelman Syndrome can be alleviated

Several models are used to explain how the Ube3a-ATS transcript silences paternal UBE3A expression: the collision model, the RNA-DNA interaction model, and the double stranded RNA interference model. See, e.g., Mabb et al., 2011. The collision model suggests that the sense and antisense templates overlap for the Ube3a-ATS antisense transcript and UBE3A, and the RNA polymerases transcribing each of these templates collide head-on, eventually pushing the RNA polymerase transcribing UBE3A off, resulting in incomplete UBE3A transcription. Thus, the longer the region of overlap, the more likely collision, and therefore suppression of UBE3A transcription, will occur. The RNA-DNA model suggests that the Ube3a-ATS transcript binds to the UBE3A DNA and induces histone modification. Histone modifications in turn interfere with transcription of UBE3A. In double stranded RNA interference model, the Ube3a-ATS transcript binds directly to UBE3A, creating double-stranded RNA that can trigger RNA interference.

Additionally, trans silencing has been proposed to occur via an interaction between the paternal Ube3a-ATS transcript and the UBE3A transcript from the maternal allele of UBE3A (Landers, M. et al., Maternal disruption of Ube3a leads to increased expression of Ube3a-ATS in trans, Nucleic Acids Res. 33:3976-84 (2005)). This interaction destabilizes both transcripts. But when maternal UBE3A is suppressed, the Ube3a-ATS transcript is stabilized.

In certain embodiments, the guide RNA targets the Ube3a-ATS downstream of the coding region of a Snord115 transcript. In some embodiments, the guide RNA targets the Ube3a-ATS downstream of the coding region of a Snord116 transcript. In some embodiments, the guide RNA targets the Ube3a-ATS upstream of a sequence complementary to a polynucleotide in a gene encoding a protein isoform of UBE3A. In some embodiments, the protein isoform of UBE3A comprises SEQ ID NO: 1, SEQ ID NO: 2, or SEQ ID NO: 3.

In certain embodiments, the guide RNA targets the Ube3a-ATS transcript at a sequence complementary to a polynucleotide sequence in a UBE3A/SNORD region. In some instances, the UBE3A/SNORD region is the entire DNA sequence between the UBE3A coding region and the coding region for a Snord transcript. In certain embodiments, the UBE3A/SNORD region comprises 1 kb to 3 kb of DNA sequence between the UBE3A coding region and the coding region for a Snord transcript. In some embodiments, the 1 kb to 3 kb of DNA sequence comprises one or more DNaseI hypersensitive sites (to optimize accessibility), a sequence encoding a sequence found in a Ube3a-ATS exon, a sequence encoding a sequence that is repeated in a Ube3a-ATS (to enable multiple site targeting with a single Cas13 guide RNA), a sequence unlikely to adopt a structure that reduces the ability of Cas13 to cleave the sequence, and/or a highly favored Ube3a-ATS coding region target sequence using a Cas13 site selection algorithm (e.g., a green score in ChopChop at https://chopchop.cbu.uib.no, or a guide score >50% in cas13design at https://cas13design.nygenome.org/).

In some aspects, the polynucleotide sequence in a UBE3A/SNORD region is a sequence within SEQ ID NO: 4, SEQ ID NO: 5, SEQ ID NO: 6, SEQ ID NO: 7, SEQ ID NO: 8, SEQ ID NO: 9, or SEQ ID NO: 10. In some aspects, the polynucleotide sequence in a UBE3A/SNORD region is a sequence within SEQ ID NO: 4, SEQ ID NO: 5, SEQ ID NO: 6, or SEQ ID NO: 7. In some aspects, the polynucleotide sequence in a UBE3A/SNORD region is a sequence within SEQ ID NO: 11, SEQ ID NO: 12, SEQ ID NO: 13, or SEQ ID NO: 14.

In some instances, the compositions described herein comprise a gene regulating moiety to regulate the expression of RNA transcripts. In some embodiments, the gene regulating moiety comprises a CRISPR-Cas polypeptide. The term “Cas polypeptide” or “Cas nuclease” refers to a Clustered Regularly Interspaced Short Palindromic Repeats-associated polypeptide or nuclease that cleaves RNA or DNA at sites specified by a guide sequence contained within a crRNA transcript. In some embodiments, the gene regulating moiety can be, for example, type VI CRISPR-associated RNA binding proteins, or a functional fragment thereof. Cas polypeptides suitable for use with the present disclosure can include Cas13; any derivative thereof; any variant thereof, or any fragment thereof. In some embodiments, Cas13 can include, but is not limited to, Cas13a (or C2C2), Cas13b, Cas13c, Cas 13d (e.g., CasRx), Cas13X, or Cas13Y. See, e.g., Liu and Pei (2022) “Insights Gained from RNA Editing Targeted by the CRISPR-Cas13 Family,” Intl J of Mol Sci, 23:11400.

In some embodiments, the Cas13 protein is less than 3 kb in size and thus can be easily packaged into a known low immunogenicity vector such as adeno-associated virus vector (AAV). Cas13 proteins edit at the RNA level, so their use rarely results in permanent genetic damage to cells. Furthermore, in some embodiments, the Cas13 protein is a variant of Cas13 that can produce targeted RNA degradation with minimal off-target effects or cell growth arrest. In some embodiments, the Cas13 protein is a variant of Cas13d comprising at least one amino acid change in the N1, N2, N3, or N15 segment of the Cas13d protein sequence relative to a wildtype Cas 13d protein sequence. In some embodiments, the Cas13 protein is Cas13d-N2V8. (see, Tong et al. (2023) “High-fidelity Cas13 variants for targeted RNA degradation with minimal collateral effect,” Nature Biotechnology, 41, 108-119). In some embodiments, the Cas13 protein is Cas13X-M17V6. CRISPR/Cas can be DNA and/or RNA cleaving or can exhibit reduced cleavage activity. The gene regulating moiety can be configured to complex with at least one heterologous RNA polynucleotide.

Any suitable nuclease (e.g., endonuclease) can be used as the gene regulating moiety. Suitable nucleases include, but are not limited to, type VI CRISPR-associated (Cas) polypeptides, or any functional fragment thereof, any derivative thereof, any variant thereof; and any fragment thereof.

A gene regulating moiety as disclosed herein can be coupled (e.g., linked or fused) to additional peptide sequences which are not involved in regulating gene expression, for example linker sequences, localization sequences, etc. The term “localization sequence,” as used herein, refers to a nucleotide sequence and the corresponding amino acid sequence which encodes a targeting polypeptide which mediates the localization (or retention) of a protein to a sub-cellular location, e.g., plasma membrane or membrane of a given organelle, nucleus, cytosol, mitochondria, endoplasmic reticulum (ER), Golgi, chloroplast, apoplast, peroxisome or other organelle. For example, a localization sequence can direct a protein (e.g., a receptor polypeptide or an adaptor polypeptide) to a nucleus utilizing a nuclear localization signal (NLS); outside of a nucleus of a cell, for example to the cytoplasm, utilizing a nuclear export signal (NES); mitochondria utilizing a mitochondrial targeting signal; the endoplasmic reticulum (ER) utilizing an ER-retention signal; a peroxisome utilizing a peroxisomal targeting signal; plasma membrane utilizing a membrane localization signal; or combinations thereof.

In some embodiments, the gene regulating moiety can be complexed with the at least one heterologous nucleic acid polynucleotide as described herein. In some embodiments, the at least one heterologous nucleic acid polynucleotide can be heterologous RNA polynucleotide. In some embodiments, the gene regulating moiety can be complexed with at least one heterologous RNA polynucleotide.

In some cases, the complexing with the at least one heterologous RNA polynucleotide direct and target the gene regulating moiety to the targeted transcript. In some cases, the complexing with the at least one heterologous RNA polynucleotide directs and targets the gene regulating moiety to a sequence in the targeted transcript for cleavage or degradation. In some cases, the complexing with the at least one heterologous RNA polynucleotide directs and targets the gene regulating moiety to a sequence in the targeted transcript where the gene regulating moiety reduces the expression of the targeted transcript in a cell. Once Cas13 is guided to its target RNA via the crRNA, in some instances it also unleashes a non-specific RNase activity against any nearby transcripts, regardless of their complementarity to the crRNA spacer.

In some embodiments, the gene regulating moiety can reduce the expression of the targeted transcript sequence between about 0.1 fold to about 10,000 fold. In some embodiments, the gene regulating moiety can reduce the expression of the targeted transcript sequence between about 0.1 fold to about 0.2 fold, about 0.1 fold to about 0.5 fold, about 0.1 fold to about 1 fold, about 0.1 fold to about 2 fold, about 0.1 fold to about 5 fold, about 0.1 fold to about 10 fold, about 0.1 fold to about 20 fold, about 0.1 fold to about 50 fold, about 0.1 fold to about 100 fold, about 0.1 fold to about 1,000 fold, about 0.1 fold to about 10,000 fold, about 0.2 fold to about 0.5 fold, about 0.2 fold to about 1 fold, about 0.2 fold to about 2 fold, about 0.2 fold to about 5 fold, about 0.2 fold to about 10 fold, about 0.2 fold to about 20 fold, about 0.2 fold to about 50 fold, about 0.2 fold to about 100 fold, about 0.2 fold to about 1,000 fold, about 0.2 fold to about 10,000 fold, about 0.5 fold to about 1 fold, about 0.5 fold to about 2 fold, about 0.5 fold to about 5 fold, about 0.5 fold to about 10 fold, about 0.5 fold to about 20 fold, about 0.5 fold to about 50 fold, about 0.5 fold to about 100 fold, about 0.5 fold to about 1,000 fold, about 0.5 fold to about 10,000 fold, about 1 fold to about 2 fold, about 1 fold to about 5 fold, about 1 fold to about 10 fold, about 1 fold to about 20 fold, about 1 fold to about 50 fold, about 1 fold to about 100 fold, about 1 fold to about 1,000 fold, about 1 fold to about 10,000 fold, about 2 fold to about 5 fold, about 2 fold to about 10 fold, about 2 fold to about 20 fold, about 2 fold to about 50 fold, about 2 fold to about 100 fold, about 2 fold to about 1,000 fold, about 2 fold to about 10,000 fold, about 5 fold to about 10 fold, about 5 fold to about 20 fold, about 5 fold to about 50 fold, about 5 fold to about 100 fold, about 5 fold to about 1,000 fold, about 5 fold to about 10,000 fold, about 10 fold to about 20 fold, about 10 fold to about 50 fold, about 10 fold to about 100 fold, about 10 fold to about 1,000 fold, about 10 fold to about 10,000 fold, about 20 fold to about 50 fold, about 20 fold to about 100 fold, about 20 fold to about 1,000 fold, about 20 fold to about 10,000 fold, about 50 fold to about 100 fold, about 50 fold to about 1,000 fold, about 50 fold to about 10,000 fold, about 100 fold to about 1,000 fold, about 100 fold to about 10,000 fold, or about 1,000 fold to about 10,000 fold. In some embodiments, the gene regulating moiety can reduce the expression of the targeted transcript sequence between about 0.1 fold, about 0.2 fold, about 0.5 fold, about 1 fold, about 2 fold, about 5 fold, about 10 fold, about 20 fold, about 50 fold, about 100 fold, about 1,000 fold, or about 10,000 fold. In some embodiments, the gene regulating moiety can reduce the expression of the targeted transcript sequence between at least about 0.1 fold, about 0.2 fold, about 0.5 fold, about 1 fold, about 2 fold, about 5 fold, about 10 fold, about 20 fold, about 50 fold, about 100 fold, or about 1,000 fold. In some embodiments, the gene regulating moiety can reduce the expression of the targeted transcript sequence between at most about 0.2 fold, about 0.5 fold, about 1 fold, about 2 fold, about 5 fold, about 10 fold, about 20 fold, about 50 fold, about 100 fold, about 1,000 fold, or about 10,000 fold.

In some embodiments, the gene regulating moiety can reduce the expression of the targeted transcript sequence by at least about 0.1 fold to about 10,000 fold. In some embodiments, the gene regulating moiety can reduce the expression of the targeted transcript sequence by at least about 0.1 fold to about 0.2 fold, about 0.1 fold to about 0.5 fold, about 0.1 fold to about 1 fold, about 0.1 fold to about 2 fold, about 0.1 fold to about 5 fold, about 0.1 fold to about 10 fold, about 0.1 fold to about 20 fold, about 0.1 fold to about 50 fold, about 0.1 fold to about 100 fold, about 0.1 fold to about 1,000 fold, about 0.1 fold to about 10,000 fold, about 0.2 fold to about 0.5 fold, about 0.2 fold to about 1 fold, about 0.2 fold to about 2 fold, about 0.2 fold to about 5 fold, about 0.2 fold to about 10 fold, about 0.2 fold to about 20 fold, about 0.2 fold to about 50 fold, about 0.2 fold to about 100 fold, about 0.2 fold to about 1,000 fold, about 0.2 fold to about 10,000 fold, about 0.5 fold to about 1 fold, about 0.5 fold to about 2 fold, about 0.5 fold to about 5 fold, about 0.5 fold to about 10 fold, about 0.5 fold to about 20 fold, about 0.5 fold to about 50 fold, about 0.5 fold to about 100 fold, about 0.5 fold to about 1,000 fold, about 0.5 fold to about 10,000 fold, about 1 fold to about 2 fold, about 1 fold to about 5 fold, about 1 fold to about 10 fold, about 1 fold to about 20 fold, about 1 fold to about 50 fold, about 1 fold to about 100 fold, about 1 fold to about 1,000 fold, about 1 fold to about 10,000 fold, about 2 fold to about 5 fold, about 2 fold to about 10 fold, about 2 fold to about 20 fold, about 2 fold to about 50 fold, about 2 fold to about 100 fold, about 2 fold to about 1,000 fold, about 2 fold to about 10,000 fold, about 5 fold to about 10 fold, about 5 fold to about 20 fold, about 5 fold to about 50 fold, about 5 fold to about 100 fold, about 5 fold to about 1,000 fold, about 5 fold to about 10,000 fold, about 10 fold to about 20 fold, about 10 fold to about 50 fold, about 10 fold to about 100 fold, about 10 fold to about 1,000 fold, about 10 fold to about 10,000 fold, about 20 fold to about 50 fold, about 20 fold to about 100 fold, about 20 fold to about 1,000 fold, about 20 fold to about 10,000 fold, about 50 fold to about 100 fold, about 50 fold to about 1,000 fold, about 50 fold to about 10,000 fold, about 100 fold to about 1,000 fold, about 100 fold to about 10,000 fold, or about 1,000 fold to about 10,000 fold. In some embodiments, the gene regulating moiety can reduce expression of the targeted transcript sequence by at least about 0.1 fold, about 0.2 fold, about 0.5 fold, about 1 fold, about 2 fold, about 5 fold, about 10 fold, about 20 fold, about 50 fold, about 100 fold, about 1,000 fold, or about 10,000 fold. In some embodiments, the gene regulating moiety can reduce the expression of the targeted transcript sequence by at least at least about 0.1 fold, about 0.2 fold, about 0.5 fold, about 1 fold, about 2 fold, about 5 fold, about 10 fold, about 20 fold, about 50 fold, about 100 fold, or about 1,000 fold. In some embodiments, the gene regulating moiety can reduce the expression of the targeted transcript sequence by at least at most about 0.2 fold, about 0.5 fold, about 1 fold, about 2 fold, about 5 fold, about 10 fold, about 20 fold, about 50 fold, about 100 fold, about 1,000 fold, or about 10,000 fold.

In some embodiments, the gene regulating moiety can reduce the expression of the targeted transcript sequence by at most about 0.1 fold to about 10,000 fold. In some embodiments, the gene regulating moiety can reduce the expression of the RNA target sequence by at most about 0.1 fold to about 0.2 fold, about 0.1 fold to about 0.5 fold, about 0.1 fold to about 1 fold, about 0.1 fold to about 2 fold, about 0.1 fold to about 5 fold, about 0.1 fold to about 10 fold, about 0.1 fold to about 20 fold, about 0.1 fold to about 50 fold, about 0.1 fold to about 100 fold, about 0.1 fold to about 1,000 fold, about 0.1 fold to about 10,000 fold, about 0.2 fold to about 0.5 fold, about 0.2 fold to about 1 fold, about 0.2 fold to about 2 fold, about 0.2 fold to about 5 fold, about 0.2 fold to about 10 fold, about 0.2 fold to about 20 fold, about 0.2 fold to about 50 fold, about 0.2 fold to about 100 fold, about 0.2 fold to about 1,000 fold, about 0.2 fold to about 10,000 fold, about 0.5 fold to about 1 fold, about 0.5 fold to about 2 fold, about 0.5 fold to about 5 fold, about 0.5 fold to about 10 fold, about 0.5 fold to about 20 fold, about 0.5 fold to about 50 fold, about 0.5 fold to about 100 fold, about 0.5 fold to about 1,000 fold, about 0.5 fold to about 10,000 fold, about 1 fold to about 2 fold, about 1 fold to about 5 fold, about 1 fold to about 10 fold, about 1 fold to about 20 fold, about 1 fold to about 50 fold, about 1 fold to about 100 fold, about 1 fold to about 1,000 fold, about 1 fold to about 10,000 fold, about 2 fold to about 5 fold, about 2 fold to about 10 fold, about 2 fold to about 20 fold, about 2 fold to about 50 fold, about 2 fold to about 100 fold, about 2 fold to about 1,000 fold, about 2 fold to about 10,000 fold, about 5 fold to about 10 fold, about 5 fold to about 20 fold, about 5 fold to about 50 fold, about 5 fold to about 100 fold, about 5 fold to about 1,000 fold, about 5 fold to about 10,000 fold, about 10 fold to about 20 fold, about 10 fold to about 50 fold, about 10 fold to about 100 fold, about 10 fold to about 1,000 fold, about 10 fold to about 10,000 fold, about 20 fold to about 50 fold, about 20 fold to about 100 fold, about 20 fold to about 1,000 fold, about 20 fold to about 10,000 fold, about 50 fold to about 100 fold, about 50 fold to about 1,000 fold, about 50 fold to about 10,000 fold, about 100 fold to about 1,000 fold, about 100 fold to about 10,000 fold, or about 1,000 fold to about 10,000 fold. In some embodiments, the gene regulating moiety can reduce the expression of the targeted transcript sequence by at most about 0.1 fold, about 0.2 fold, about 0.5 fold, about 1 fold, about 2 fold, about 5 fold, about 10 fold, about 20 fold, about 50 fold, about 100 fold, about 1,000 fold, or about 10,000 fold. In some embodiments, the gene regulating moiety can reduce the expression of the targeted transcript sequence by at most at least about 0.1 fold, about 0.2 fold, about 0.5 fold, about 1 fold, about 2 fold, about 5 fold, about 10 fold, about 20 fold, about 50 fold, about 100 fold, or about 1,000 fold. In some embodiments, the gene regulating moiety can reduce the expression of the targeted transcript sequence by at most about 0.2 fold, about 0.5 fold, about 1 fold, about 2 fold, about 5 fold, about 10 fold, about 20 fold, about 50 fold, about 100 fold, about 1,000 fold, or about 10,000 fold. In some embodiments, the targeted transcript sequence is a sequence complementary to a polynucleotide sequence in a UBE3A/SNORD region. In some embodiments, the targeted transcript sequence is downstream of a sequence complementary to a coding region of a protein isoform of UBE3A.

In some embodiments, the heterologous RNA polynucleotide is a crRNA (CRISPR RNA) guide RNA. Guide RNAs can be introduced into cells expressing an engineered chimeric receptor polypeptide. In some cases, such cells contain one or more different guide RNAs that target the same nucleic acid. In other cases, guide RNAs target different nucleic acids in the cell. The Cas13 guide RNA may comprise about 60-66 nucleotides and encode both a short hairpin that associates with Cas polypeptide and a 21-32 nucleotide spacer that provides target specificity by being complementary to an RNA target sequence.

In certain embodiments, the polynucleotide in a UBE3A/SNORD region is about 21 nucleotides to about 32 nucleotides long. In certain embodiments, the Cas13 protein comprises a Cas13b protein or a variant thereof. In certain embodiments, the Cas13 protein is Cas13d or a variant thereof. Cas13d is about 20% (190-300 aa) smaller than Cas13a-Cas13c. In certain embodiments, the Cas13 protein is Cas13X or a variant thereof. Cas13X is overall less than 1,000 amino acids in size. Using Cas13d or Cas13X or a variant thereof (e.g., Cas13d-N2V8 or Cas13X-M17V6) is therefore advantageous for use in vectors with limited capacity. In certain embodiments, the Cas13 protein and guide RNA further comprise a pharmaceutically acceptable carrier.

In certain aspects, the disclosure herein provides a vector comprising a polynucleotide sequence encoding a Cas13 protein and a polynucleotide sequence encoding a guide RNA that targets a Ube3a-ATS transcript. A vector is contemplated to have the necessary functional elements that direct and regulate transcription of the inserted nucleic acid. These functional elements include, but are not limited to, a promoter, regions upstream or downstream of the promoter, such as enhancers that may regulate the transcriptional activity of the promoter, an origin of replication, appropriate restriction sites to facilitate cloning of inserts adjacent to the promoter, antibiotic resistance genes or other markers that can serve to select for cells containing the vector or the vector containing the insert, RNA splice junctions, a transcription termination region, or any other region that may serve to facilitate the expression of the inserted gene or hybrid gene (See generally, Sambrook et al. Molecular Cloning: A Laboratory Manual, 4th ed., Cold Spring Harbor Laboratory Press, Cold Spring Harbor, 2012). The vector, for example, can be a plasmid.

In certain embodiments, the guide RNA targets the Ube3a-ATS transcript downstream of the coding region of a Snord115 transcript. In some embodiments, the guide RNA targets the Ube3a-ATS downstream of the coding region of a Snord116 transcript. In some embodiments, the guide RNA targets the Ube3a-ATS upstream of a sequence complementary to a polynucleotide in a gene encoding a protein isoform of UBE3A. In some embodiments, the protein isoform of UBE3A comprises SEQ ID NO: 1, SEQ ID NO: 2, or SEQ ID NO: 3. In certain embodiments, the guide RNA targets the Ube3a-ATS transcript at a sequence complementary to a polynucleotide sequence in a UBE3A/SNORD region. In some aspects, the polynucleotide sequence in a UBE3A/SNORD region is a sequence within SEQ ID NO: 4, SEQ ID NO: 5, SEQ ID NO: 6, SEQ ID NO: 7, SEQ ID NO: 8, SEQ ID NO: 9, or SEQ ID NO: 10. In some aspects, the polynucleotide sequence in a UBE3A/SNORD region is a sequence within SEQ ID NO: 4, SEQ ID NO: 5, SEQ ID NO: 6, or SEQ ID NO: 7. In some aspects, the polynucleotide sequence in a UBE3A/SNORD region is a sequence within SEQ ID NO: 11, SEQ ID NO: 12, SEQ ID NO: 13, or SEQ ID NO: 14. In certain embodiments, the polynucleotide in a UBE3A/SNORD region is about 21 nucleotides to about 32 nucleotides long. In certain embodiments, the Cas13 protein comprises a Cas13b protein or a variant thereof. In certain embodiments, the Cas13 protein comprises a Cas13d protein or a variant thereof. In some embodiments, the Cas13 protein comprises a Cas13X protein or variant thereof. In some embodiments, the Cas13 protein is Cas13d-N2V8 or Cas13X-M17V6.

In some embodiments, the vector comprises a polynucleotide sequence encoding a Cas13 protein that is codon optimized for expression in a human cell. In some embodiments, the vector comprises a polynucleotide sequence encoding a Cas13 protein that is codon optimized for expression in a rat cell.

In some embodiments, the polynucleotide sequence encoding the guide RNA comprises a U6::crRNA cassette. The U6 cassette comprises a polynucleotide sequence that encodes a crRNA and at least one U6 promoter. U6 is a type III RNA polymerase III promoter that can drive expression of single-stranded RNA.

In some embodiments, the vector contains a promoter element that directs transgene expression to the brain and/or spinal cord. In some embodiments, the promoter element can be neuron-specific. In some embodiments, the promoter element allows for continuous expression at least in part of the brain and/or spinal cord. In some embodiments, the promoter comprises the promoter from the human SYN1 gene (hSYN1). SYN1 encodes synapsin 1, a neuron-specific phosphoprotein associated with epilepsy, various learning disabilities, behavior disorders, and intellectual development disorders.

In some instances, the promoter comprises a promoter or a fragment of a promoter from a gene known to be expressed in the central nervous system (CNS). Such a gene could be, for example, glutamic acid decarboxylase (GAD67), homeobox Dlx5/6, glutamate receptor (GluR1), preprotachykinin 1 (Tac1), neuron-specific enolase (NSE), dopaminergic receptor 1 (Drdh1a), tubulin α1 (Ta1), human U1 snRNA (U1), mouse α-calcium-calmodulin dependent kinase II (Camk2a(long) or Camk2a(short)), mouse tyrosine hydroxylase (Th), mouse Hsp68, or methyl-CpG-binding protein-2 (MeCP2), human glial fibrillary acidic protein (GFAP (long) or GFAP (short)), mouse ionized calcium binding adapter molecule 1 (Iba1), or mouse prion protein (Prnp). (See, e.g., Delzor et al., Restricted Transgene Expression in the Brain with Cell-type Specific Neuronal Promoters, Human Gene Therapy Methods, 23:242-54 (2012); Kugler et al, Neuron-Specific Expression of Therapeutic Proteins: Evaluation of Different Cellular Promoters in Recombinant Adenoviral Vectors, Molecular and Cellular Neuroscience 17:78-96 (2001); and Vector Builder at https: en.vectorbuilder.com/resources/vector-component/promoter.html). In some embodiments, the promoter comprises a promoter known to function as a viral promoter, such as the human cytomegalovirus (CMV) promoter or a hybrid thereof such as the hybrid chicken β-actin (CBA)/CMV promoter CBh (Gray et al., Optimizing Promoters for Recombinant Adeno-Associated Virus-Mediated Gene Expression in the Peripheral and Central Nervous System Using Self-Complementary Vectors, Human Gene Therapy, 22:1143-53 (2011)). In some embodiments, the vector comprises cis-regulatory elements to regulate or boost expression of the transgene.

In some embodiments, the vector is an adeno-associated virus (AAV) vector. AAV vectors are commonly used to deliver therapeutic molecules because there are several advantages to using them including nonpathogenicity, inability to incur an immune response, ability to produce robust expression, and ability to be expressed in varied tissues within the body. Nonetheless, AAV vector typically have packaging capacity limits of around 5 kb, limiting the number and types of genes that they can encode.

In some embodiments, the AAV vector is selected from a wild-type AAV serotype 1 (AAV1), wild-type AAV serotype 2 (AAV2), wild-type AAV serotype 3 (AAV3), wild-type AAV serotype 4 (AAV4), wild-type AAV serotype 5 (AAV5), wild-type AAV serotype 6 (AAV6), wild-type AAV serotype 7 (AAV7), wild-type AAV serotype 8 (AAV8), wild-type AAV serotype 9 (AAV9), wild-type AAV serotype 10 (AAV10), wild-type AAV serotype 11 (AAV11), wild-type AAV serotype 12 (AAV12), a variant thereof, and any shuffled chimera thereof. In some instances, the homologous donor AAV vector has at least about 90% sequence identity to any one selected from the group consisting of an AAV1, AAV2, AAV3, AAV4, AAV3, AAV5, AAV6, AAV7, AAV8, AAV9, AAV10, AAV11 and AAV12.

Previous studies showed that genes encoded by an AAV9 vector can be expressed in the central nervous system and peripheral tissues after intravenous or intrathecal injection. This is partly due to the ability of this vector to cross the blood-brain barrier and transduce astrocytes and neurons. An alternative AAV vector, AAV-PHP.eB, is often used for research on the nervous system in mice because, like AAV9, it is able to cross the blood-brain barrier after various routes of administration. However, its efficacy is limited to only a subset of mouse strains, including C57BL/6J and B6C3.

In some instances, the vector is an AAV vector that can cross the blood-brain barrier. In some embodiments, the AAV vector encodes an AAV capsid that allows for enhanced transduction to cells in the central nervous system. In some embodiments, the vector is AAV9. In some instances, the AAV vector is an AAV9 or an AAV9 variant having at least 95% sequence identity to wild-type AAV9, e.g., 95%, 96%, 97%, 98%, 99%, or 100% sequence identity to wild-type AAV9.

In some embodiments, the disclosure comprises a cell containing a vector as described herein. In some embodiments, the cell is a neuron or a stem cell. A vector can be introduced into a cell through a manner suitable for subsequent expression of the nucleic acid, such as transfection. As used herein, the phrase “introduced” in the context of introducing a nucleic acid into a cell refers to the translocation of the nucleic acid sequence from outside a cell to inside the cell. In some cases, introducing refers to translocation of the nucleic acid from outside the cell to inside the nucleus of the cell. The method of introduction is largely dictated by the targeted cell type. Exemplary methods include chemical methods such as using cationic polymers, CaPO₄ precipitation, liposome fusion, and cationic liposomes, dextran-mediated transfection, polybrene-mediated transfection, physical methods such as electroporation, nucleoporation, laser-based transfection, protoplast fusion, and direct microinjection, and biological transduction, such as via viral infection. Various methods of translocation are contemplated, including but not limited to, electroporation, nanoparticle delivery, viral delivery, contact with nanowires or nanotubes, receptor mediated internalization, translocation via cell penetrating peptides, liposome mediated translocation, DEAE dextran, lipofectamine, calcium phosphate or any method now known or identified in the future for introduction of nucleic acids into prokaryotic or eukaryotic cellular hosts.

In certain embodiments, the disclosure provides a method for de-repressing UBE3A expression in a cell, comprising contacting a cell with any of the compositions described herein, or transfecting a cell with the any of the vectors described herein. “Contacting” a cell may include addition of a composition to a cell culture in vitro, or administering a composition to a subject (e.g., in conjunction with a pharmaceutical composition as described above). In certain embodiments, the disclosure provides a method for cleaving a UBE3A antisense RNA in a cell, comprising contacting the cell with any of the compositions described herein, or transfecting the cell with the any of the vectors described herein. In certain embodiments of a method for de-repressing UBE3A expression in a cell, the cell is a neuron or a stem cell.

In some embodiments, the disclosure provides a method for treating Angelman Syndrome in a subject in need thereof, comprising administering to the subject a therapeutically effective amount of a composition described herein, or a therapeutically effective amount of a vector described herein, or an effective amount of a population of one of the cells described herein. In certain embodiments, the therapeutically effective amount is an amount sufficient to de-repress the expression of all three major protein isoforms of UBE3A. In certain embodiments, the route of administration is at least one of intraparenchymal, intranasal, intracerebroventrical, intracisternal, intrathecal, intravenous, intramuscular, via the spinal cord, via the eye, or via the cochlea. In certain embodiments, the route of administration is at least one of intrathecal, intracerebroventrical, or via the intracisterna magna.

As used herein, the terms “effective amount” and “therapeutically effective amount” refer to a dose of a compound that produces therapeutic effects for which it is administered. The exact dose will depend on the therapeutic context and objectives, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); Goodman & Gilman's The Pharmacological Basis of Therapeutics, 11^{th} Edition, 2006, Brunton, Ed., McGraw-Hill; and Remington: The Science and Practice of Pharmacy, 21^{st} Edition, 2005, Hendrickson, Ed., Lippincott, Williams & Wilkins).

The clinician can select the frequency of dosing, taking into account the pharmacokinetic parameters of the fusion protein or modified protein in the formulation used. In certain embodiments, a clinician administers the composition until a dosage is reached that achieves the desired effect. In certain embodiments, the composition can therefore be administered as a single dose or as two or more doses (which may or may not contain the same amount of the desired molecule) over time, or as a continuous infusion via, for example, an implantation device or catheter. Further refinement of the appropriate dosage is routinely made by those of ordinary skill in the art and is within the ambit of tasks routinely performed by them. In certain embodiments, appropriate dosages can be ascertained through use of appropriate dose-response data. Dosing considerations and administration are discussed further below.

It should be understood, however, that the specific dose level and frequency of dosage for any particular patient may be varied and may depend upon a variety of factors including the activity of the specific compound employed, the metabolic stability and length of action of that compound, the age, body weight, hereditary characteristics, general health, sex, diet, mode and time of administration, rate of excretion, drug combination, the severity of the particular condition, and the host undergoing therapy. The clinician can also titer the dosage and modify the route of administration to obtain the optimal therapeutic effect.

As used herein, the term “unit dosage form” refers to physically discrete units suitable as unitary dosages for humans and other mammals, each unit containing a predetermined quantity of a therapeutic agent calculated to produce the desired onset, tolerability, and/or therapeutic effects, in association with a suitable pharmaceutical excipient. In addition, more concentrated dosage forms may be prepared, from which the more dilute unit dosage forms may then be produced. The more concentrated dosage forms thus will contain substantially more than, e.g., at least 1, at least 2, at least 3, at least 4, at least 5, at least 6, at least 7, at least 8, at least 9, at least 10, or more times the amount of the therapeutic compound.

Methods for preparing such dosage forms are known to those skilled in the art (see, e.g., REMINGTON'S PHARMACEUTICAL SCIENCES, supra). The dosage forms typically include a conventional pharmaceutical carrier or excipient and may additionally include other medicinal agents, carriers, adjuvants, diluents, tissue permeation enhancers, solubilizers, and the like. Appropriate excipients can be tailored to the particular dosage form and route of administration by methods well known in the art (see, e.g., REMINGTON'S PHARMACEUTICAL SCIENCES, supra).

In certain embodiments, expression of Cas13 and guide RNA is maintained at least 16 weeks in the subject. A number of ASO therapies for neurodevelopment disorders have been developed (see, e.g., Hill and Meisler, Antisense Oligonucleotide Therapy for Neurodevelopmental Disorders, Dev. Neurosci., 43:247-52 (2021)). In clinical trials, ASO-based therapeutics were cleared after 3-4 months, and so required at least 2 to 4 treatments a year. Mice injected with ASOs to treat a model for SCN8a encephalopathy survived 7 weeks longer than untreated mice. Id. By targeting the Ube3a-ATS antisense RNA using a Cas13 system expressed on an AAV vector, expression should be continuous, allowing the possibility of a subject needing fewer doses, potentially only a single therapeutic dose.

In some embodiments, expression of Cas13 and guide RNA is maintained at least about at least 7 weeks, about 8 weeks, about 9 weeks, about 10 weeks, about 10 weeks to about 12 weeks, about 13 weeks to about 16 weeks, about 17 weeks to about 20 weeks, about 21 weeks to about 24 weeks, about 25 weeks to about 32 weeks, about 33 weeks to about 36 weeks, about 37 weeks to about 40 weeks, about 41 weeks to about 44 weeks, about 45 weeks to about 48 weeks, about 49 weeks to about 1 year, about 1 year to about 1.5 years, about 1.5 years to about 2 years, about 2 years to about 2.5 years, about 2.5 years to about 3 years, about 3 years to about 5 years, about 5 years to about 10 years, about 10 years to about 15 years, about 15 years to about 20 years, about 20 years to about 25 years, about 25 years to about 30 years, or greater than 30 years.

The disclosure also provides a method for treating Angelman Syndrome in a subject in need thereof, wherein the subject has a mutation in the maternal UBE3A gene. AS can be traced to multiple genetic alternations at chromosome 15q11.2-q13. However, the majority of AS cases (approximately 70%) result from deletions in the maternal chromosome.

### IV. Examples

The subject matter of the present disclosure will be described in greater detail by way of specific examples. The following examples are provided to illustrate, but not to limit, the claimed subject matter.

### Example 1. Materials and Methods

Vector design. Initial studies used a human codon-optimized nuclease PspCas13b that was cloned into the backbone of PX551 plasmid (Addgene #60957) that was first excised by AgeI and NotI, followed by sequential Gibson cloning of PspCas13b and a 582 bp long G-block that added the 3×HA-polyA-U6::gRNA to the construct. The full construct used in the initial studies was AAV-ITR-Mecp2::PspCas13b-SV40NLS-3×HA-polyA;U6::gRNA-ITR.

The nuclease RfxCas13d, characterized by Konermann et al. (2018), was PCR amplified with overhangs from the Addgene plasmid (#109049) and used to replace the PspCas13b after cutting it with AgeI-HF, BamHI-HF, and StuI. PCR-amplified RfxCas13d was Gibson cloned into the backbone. To change the gRNA flanking sequence specific for RfxCas13d, another G-block was inserted by cutting the backbone with KpnI and NotI.

The promoter was switched from Mecp2 to hSyn1 using a G-block with overhangs. The full construct used in the subsequent studies was: AAV-ITR-hSyn1::RfxCas13d-SV40NLS-3×HA-polyA;U6::gRNA-ITR. The initial gRNA used for both nucleases was designed by searching for the best scoring guide using this software: http://bioinfolab.miamioh.edu/CRISPR-RT/interface/C2c2.php in the general area as the following antisense oligonucleotide: ASO A, 5′-GATCCATTTGTGTTAAGCTG-3′ (Meng et al., Towards a therapy for Angelman syndrome by targeting a long non-coding RNA, Nature 19:409-12 (2015)), and selecting gRNA-100:

GATCCATTTGTGTTAAGCTGtaatgggt. The following scrambled/non-targeting gRNA was also Gibson generated: CTTCCATGACGCAGAAGTTAACACTTTCGG. Results using these Cas13 constructs were compared to a those using a zinc finger construct that targets the SNRPN promoter for Ube3a unsilencing: AAV-CBA::TatK-HA-NLS—SIZF-KRAB-CW3SL; PGK-mCherry-WPRE3-polyA (AAV-S1K-PHP.eB).

AAV packaging. Recombinant AAVs were generated by triple transfection of 293T cells. AAV-PHP.eB (Addgene, #103005) (Chan et al. 2017, Engineered AAVs for efficient noninvasive gene delivery to the central and peripheral nervous systems, Nat Neurosci 20:1172-79 (2017)) was used as the envelope plasmid). After packaging, AAV titers were measured and confirmed with subsequent qPCR. Briefly, virus titers were determined by measuring the number of DNase I-resistant vector genomes using qPCR and comparing it with the used genome plasmid as standard (Challis et al., Widespread and targeted gene expression by systemic AAV vectors: Production, purification, and administration, bioRxiv 246405 doi: https://doi.org/10.1101/246405 (2018)).

Mice. All animal experimental procedures were conducted with and approved by the Institutional Animal Care and Use Committees (IACUC) of the University of California, Davis. All animals were housed in clear plastic shoebox cages containing corn cob bedding under constant temperature (22+2° C.) and a 12-h light-dark cycle. Food and water were provided ad libitum.

C57BL/6J and Ube3a^{m+/pYFP} mice were obtained from The Jackson Laboratories. Ube 3a^{m+/pYFP} animals were generated by crossing Ube3a^{m+/pYFP} males with wild-type C57BL/6J females. PCRs to determine the genotypes of mice (i.e., Ube3a^{m+/p+} or Ube 3a^{YFP}) were performed using methods described previously (Dindot et al., The Angelman syndrome ubiquitin ligase localizes to the synapse and nucleus, and maternal deficiency results in abnormal dendritic spine morphology, Hum Mol Genet 17:111-18 (2007)).

Tissue preparation. Intravenous administration of 1×10¹¹ or 1×10¹² vector genomes (vg) of AAV-PHP.eB was performed by injection into the tail vein of adult mice (6-9 weeks of age). After allowing time for expression (3 weeks), mice were anesthetized with Isoflurane and transcardially perfused first with cold 0.1 M phosphate buffer (pH 7.4) at room temperature. The cerebral cortex, midbrain, and cerebellum were placed into 1.5 ml tubes and quickly frozen in liquid nitrogen until further processing.

RT-qPCR. Whole-brain halves were ground in liquid nitrogen before homogenization in TriZol. RNA was extracted using the Direct-zol RNA Miniprep kit (Zymo Research). 500-1000 μg total RNA were reverse transcribed using the VILO kit (Invitrogen) according to the manufacturer's instructions. Primers for quantitative PCR (qPCR) were designed using Primer3. RT-qPCR was performed in triplicate using PowerUp SYBR Green Master Mix (ThermoFisher) with the CFX384 Real-Time System C1000 Touch system (Bio-Rad). Ube3a-YFP was quantitated using the Taqman assay. Gene expression analysis was performed with gene-specific primers using 3 biological replicates. Relative target gene expression was calculated as the difference between the target gene and the GAPDH reference gene (dCq=Cq[target]-Cq[GAPDH]). Gene expression results are indicated as fold change to a reference sample, using the ddCq method. Multiple unpaired t-tests or ANOVA were used to determine statistical significance for individual loci.

Western Blot analysis. Flash-frozen brain hemispheres were ground in liquid nitrogen. Protein was extracted with the RIPA buffer. Protein lysates were quantified using the BCA assay (Life Technologies). 20 μg protein per well was loaded onto SDS PAGE gels (PROTEAN TGX, Biorad 4-20%) and separated. Proteins were transferred to nitrocellulose membrane or PVDF overnight at 4° C. using the Wet/Tank Blotting Systems (BioRad). Total protein was stained with Ponceau S stain before blocking the membrane in 5% dry milk in TBST (20 mmol/l Tris, pH 7.5, 150 mmol/l NaCl, 0.1% Tween). Anti-GFP antibody (1:1000, NovusBio) and anti-β-actin antibody (1:10000; Sigma) were used overnight at 4° C. and were subsequently incubated with anti-mouse and anti-rabbit antibodies (1:2000, Licor). Western blots were visualized using LICOR Odyssey. Intensities of GFP protein bands were quantified using the volume box tool and normalized to intensities of β-actin.

### Example 2. Overall Approach to Using AAV-Cas13 to Alter UBE3A Expression

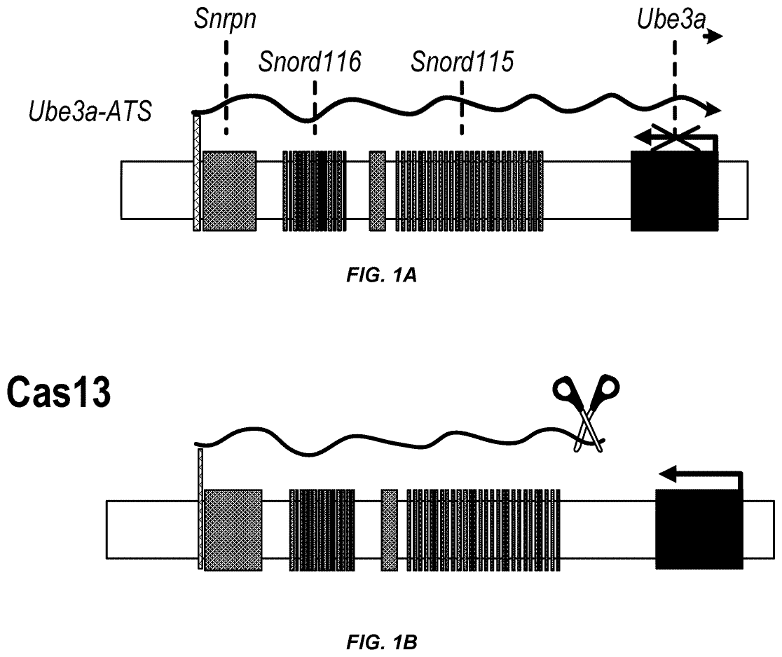

FIG. 1A illustrates the paternal UBE3A locus in Angelman disease patients. In AS, loss of the Ube3a protein in the brain is typically due to mutation or deletion of the maternal UBE3A allele. Due to genetic imprinting, the paternal UBE3A allele is present but epigenetically silenced (cross mark) by an antisense transcript (Ube3a-ATS) (FIG. 1A). As shown in FIG. 1B, Cas13 (scissors) can be targeted to the 3′ end of the antisense transcript using a crRNA that contains a spacer sequence that is complementary to the target sequence in the antisense transcript. This treatment allows permanent de-repression of UBE3A without UBE3A over-expression or loss of Snord gene expression.

### Example 3. Developing a Baseline for Measuring UBE3A Activation In Vivo

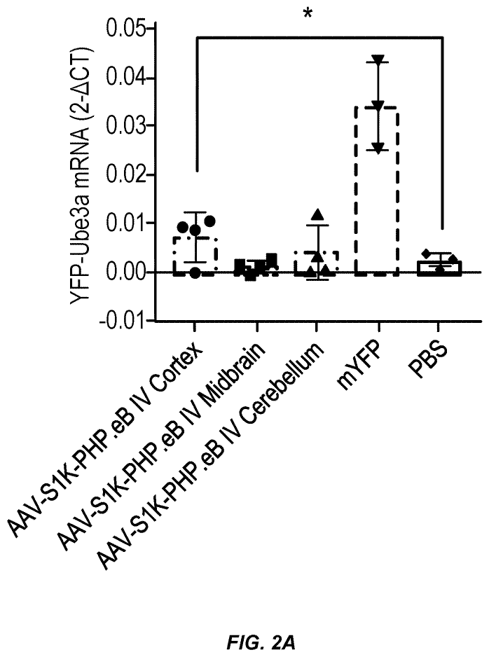

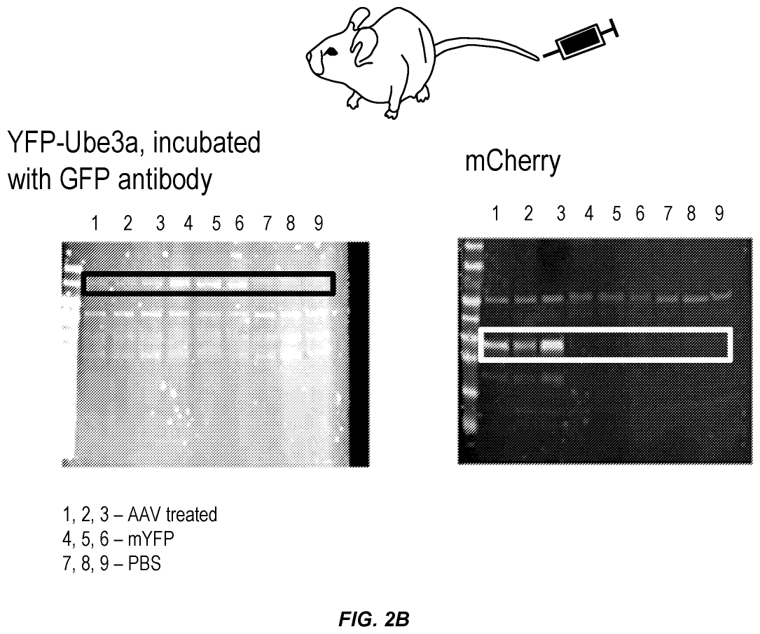

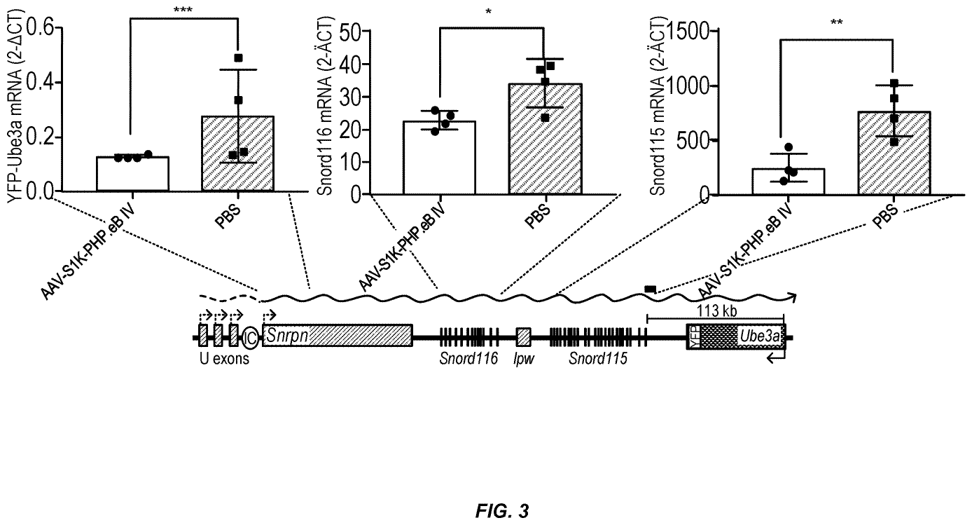

The artificial transcription factor (ATF) S1K was expressed from an AAV variant called AAV-PHP.eB that has been shown to efficiently cross the blood-brain barrier in mice. S1K is a programmable transcription factor that is able to silence the entire Ube3a-ATS transcript. AAV-S1K-PHP.eB injected intravenously in mice was able to activate Ube3a-YFP in some pYFP animals, as demonstrated by RT-qPCR (FIG. 2A) and Western blot (FIG. 2B). As determined by RT-qPCR, AAV-S1K-PHP.eB injection also decreased expression of Ube3a-ATS transcript (FIG. 3, left panel). However, it significantly decreased Snord116 and Snord115 mRNA levels as well (FIG. 3, middle and right panels). Intranasal administration, which avoids systemic distribution and thus allows less virus to be used than with intravenous injection, had similar effects, as determined by Western blot. FIGS. 4A and 4B show quantitation of intensity of YFP-Ube3a signal and images of blots stained to detect either YFP or mCherry, respectively. mCherry was used to detect AAV vector expression.

### Example 4. AAV-PHP.eB-Cas13 Increases Paternal Ube3a Protein Expression, Yet Minimally Affects Snord Transcript Expression

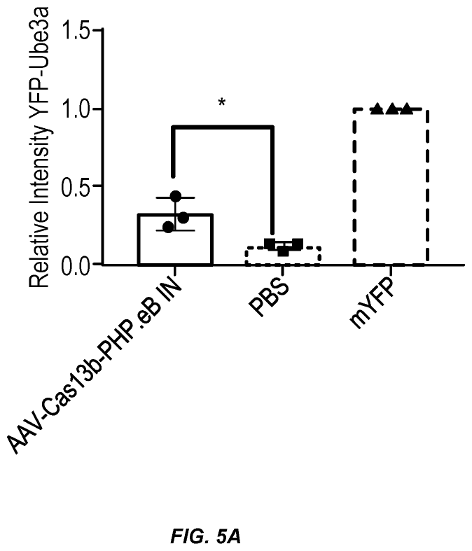

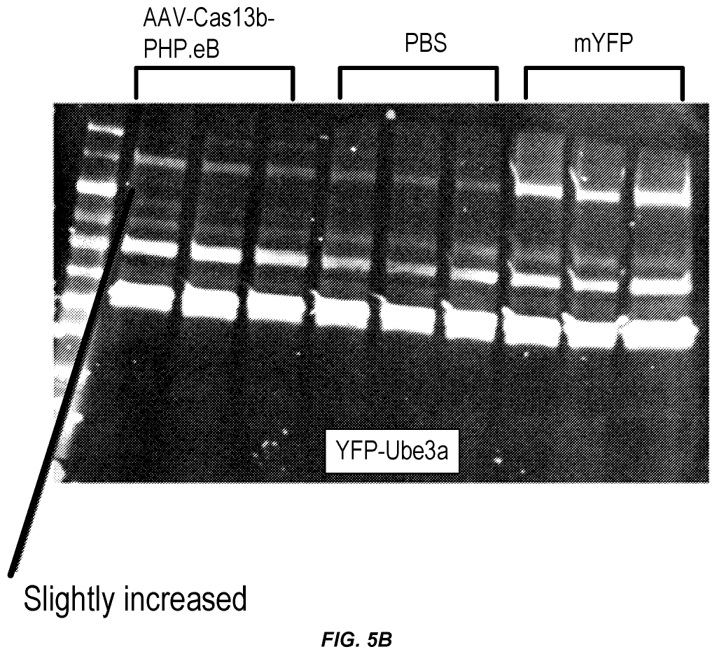

As shown in FIGS. 5A and 5B, a significant increase in paternal Ube3a-YFP protein was observed when AAV-PHP.eB-Cas13b was injected intranasally. FIG. 5A shows quantitation of intensity of YFP-Ube3a signal of the image of Western blot in FIG. 5B. Notably, Ube3a-YFP expression was similar if not greater than seen with AAV expressing ATF S1K (compare FIGS. 5A and 5B to FIGS. 2, 4A, and 4B).

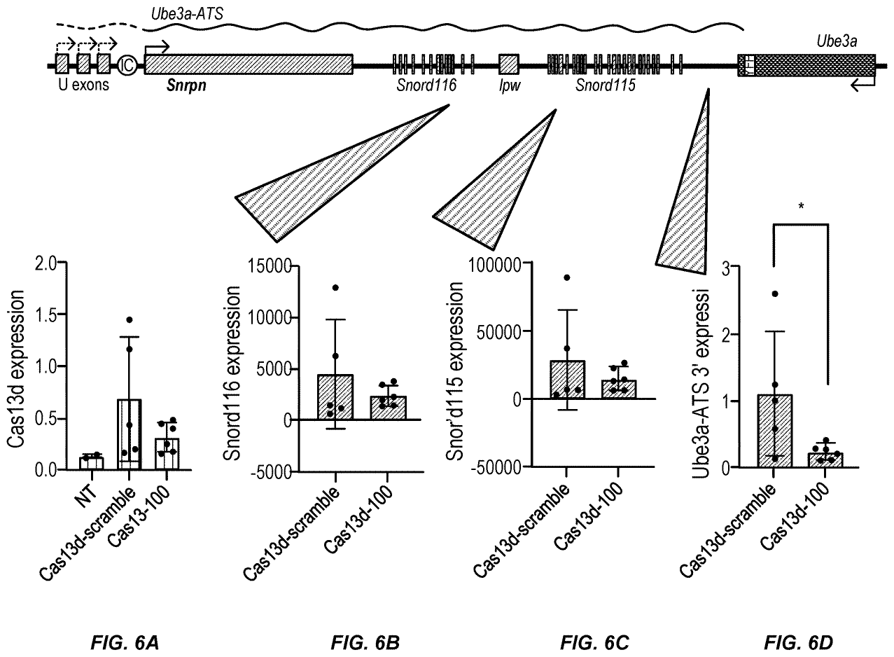

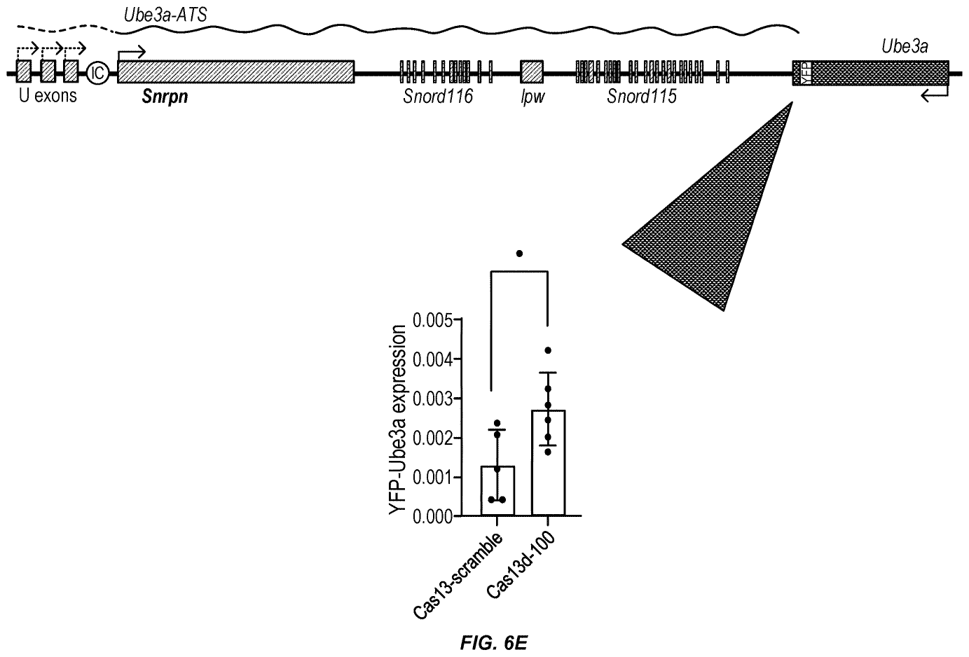

To test the effects of AAV-PHP.eB-Cas13d on Snord gene expression, reporter mice were injected by tail vein at six weeks and housed for three weeks before analyzing hippocampi by RT-qPCR. As shown in FIG. 6D, a significant increase in paternal UBE3A expression was observed (p=0.03, n=6). This correlated with loss of Ube3a-ATS RNA at the 3′ end, near the Cas13d cleavage site. (FIG. 6E). Nonetheless, Snord RNAs were not significantly affected (FIG. 6B, C) when compared to a Cas13d with a scrambled guide-RNA.Cas13d was under the control of the human SYN1 gene (hSYN1) promoter to restrict expression to neurons.

It is understood that the examples and embodiments described herein are for illustrative purposes only and that various modifications or changes in light thereof will be suggested to persons skilled in the art and are to be included within the spirit and purview of this application and scope of the appended claims. All publications, patents, patent applications, and sequence reference numbers cited herein are hereby incorporated by reference in their entirety for all purposes.

INFORMAL SEQUENCE LISTING

SEQ ID NO 1: (human ubiquitin-protein ligase E3A isoform 1)
MKRAAAKHLIERYYHQLTEGCGNEACTNEFCASCPTFLRMDNNAAAIKALELYKIN
AKLCDPHPSKKGASSAYLENSKGAPNNSCSEIKMNKKGARIDFKDVTYLTEEKVYEI
LELCREREDYSPLIRVIGRVFSSAEALVQSFRKVKQHTKEELKSLQAKDEDKDEDEKE
KAACSAAAMEEDSEASSSRIGDSSQGDNNLQKLGPDDVSVDIDAIRRVYTRLLSNEKI
ETAFLNALVYLSPNVECDLTYHNVYSRDPNYLNLFIIVMENRNLHSPEYLEMALPLF
CKAMSKLPLAAQGKLIRLWSKYNADQIRRMMETFQQLITYKVISNEFNSRNLVNDD
DAIVAASKCLKMVYYANVVGGEVDINHNEEDDEEPIPESSELTLQELLGEERRNKK
GPRVDPLETELGVKTLDCRKPLIPFEEFINEPLNEVLEMDKDYTFFKVETENKFSFMT
CPFILNAVTKNLGLYYDNRIRMYSERRITVLYSLVQGQQLNPYLRLKVRRDHIIDDAL
VRLEMIAMENPADLKKQLYVEFEGEQGVDEGGVSKEFFQLVVEEIFNPDIGMFTYDE
STKLFWFNPSSFETEGQFTLIGIVLGLAIYNNCILDVHFPMVVYRKLMGKKGTFRDLG
DSHPVLYQSLKDLLEYEGNVEDDMMITFQISQTDLFGNPMMYDLKENGDKIPITNEN
RKEFVNLYSDYILNKSVEKQFKAFRRGFHMVTNESPLKYLFRPEEIELLICGSRNLDF
QALEETTEYDGGYTRDSVLIREFWEI
VHSFTDEQKRLFLQFTTGTDRAPVGGLGKLKMIIAKNGPDTERLPTSHTCFNVLLLPE
YSSKEKLKERLLKAITYAKGFGML
SEQ ID NO 2: (human ubiquitin-protein ligase E3A isoform 2)
MEKLHQCYWKSGEPQSDDIEASRMKRAAAKHLIERYYHQLTEGCGNEACTNEFCAS
CPTFLRMDNNAAAIKALELYKINAKLCDPHPSKKGASSAYLENSKGAPNNSCSEIKM
NKKGARIDFKDVTYLTEEKVYEILELCREREDYSPLIRVIGRVFSSAEALVQSFRKVK
QHTKEELKSLQAKDEDKDEDEKEKAACSAAAMEEDSEASSSRIGDSSQGDNNLQKL
GPDDVSVDIDAIRRVYTRLLSNEKIETAFLNALVYLSPNVECDLTYHNVYSRDPNYL
NLFIIVMENRNLHSPEYLEMALPLFCKAMSKLPLAAQGKLIRLWSKYNADQIRRMM
ETFQQLITYKVISNEFNSRNLVNDDDAIVAASKCLKMVYYANVVGGEVDTNHNEED
DEEPIPESSELTLQELLGEERRNKKGPRVDPLETELGVKTLDCRKPLIPFEEFINEPLNE
VLEMDKDYTFFKVETENKFSFMTCPFILNAVTKNLGLYYDNRIRMYSERRITVLYSL
VQGQQLNPYLRLKVRRDHIIDDALVRLEMIAMENPADLKKQLYVEFEGEQGVDEGG
VSKEFFQLVVEEIFNPDIGMFTYDESTKLFWFNPSSFETEGQFTLIGIVLGLAIYNNCIL
DVHFPMVVYRKLMGKKGTFRDLGDSHPVLYQSLKDLLEYEGNVEDDMMITFQISQT
DLFGNPMMYDLKENGDKIPITNENRKEFVNLYSDYILNKSVEKQFKAFRRGFHMVT
NESPLKYLFRPEEIELLICGSRNLDFQALEETTEYDGGYTRDSVLIREFWEIVHSFTDE
QKRLFLQFTTGTDRAPVGGLGKLKMIIAKNGPDTERLPTSHTCFNVLLLPEYSSKEKL
KERLLKAITYAKGFGML
SEQ ID NO 3: (human ubiquitin-protein ligase E3A isoform 3)
MATACKRSGEPQSDDIEASRMKRAAAKHLIERYYHQLTEGCGNEACTNEFCASCPTF
LRMDNNAAAIKALELYKINAKLCDPHPSKKGASSAYLENSKGAPNNSCSEIKMNKK
GARIDFKDVTYLTEEKVYEILELCREREDYSPLIRVIGRVFSSAEALVQSFRKVKQHT
KEELKSLQAKDEDKDEDEKEKAACSAAAMEEDSEASSSRIGDSSQGDNNLQKLGPD
DVSVDIDAIRRVYTRLLSNEKIETAFLNALVYLSPNVECDLTYHNVYSRDPNYLNLFII
VMENRNLHSPEYLEMALPLFCKAMSKLPLAAQGKLIRLWSKYNADQIRRMMETFQ
QLITYKVISNEFNSRNLVNDDDAIVAASKCLKMVYYANVVGGEVDTNHNEEDDEEP
IPESSELTLQELLGEERRNKKGPRVDPLETELGVKTLDCRKPLIPFEEFINEPLNEVLE
MDKDYTFFKVETENKFSFMTCPFILNAVTKNLGLYYDNRIRMYSERRITVLYSLVQG
QQLNPYLRLKVRRDHIIDDALVRLEMIAMENPADLKKQLYVEFEGEQGVDEGGVSK
EFFQLVVEEIFNPDIGMFTYDESTKLFWFNPSSFETEGQFTLIGIVLGLAIYNNCILDVH
FPMVVYRKLMGKKGTFRDLGDSHPVLYQSLKDLLEYEGNVEDDMMITFQISQTDLF
GNPMMYDLKENGDKIPITNENRKEFVNLYSDYILNKSVEKQFKAFRRGFHMVTNESP
LKYLFRPEEIELLICGSRNLDFQALEETTEYDGGYTRDSVLIREFWEIVHSFTDEQKRL
FLQFTTGTDRAPVGGLGKLKMIIAKNGPDTERLPTSHTCFNVLLLPEYSSKEKLKERL
LKAITYAKGFGML
SEQ ID NO 4: (hum 1)
CCTGCTCTCTGTGTGGGAGGTGTTGTGTGGGGCATGGATAGGGACCCACCAGAG
ACAACACATGGCCCTCGGGCCCCAGGCTGCAATTTCTCAGTGCATGCTTGGAGAC
GGTCCTGGGCCTCCTCCATGCCCAGGGTCAGGCTGTGCAACTGTCAGGTTAGCGG
CGGCATGTGGTGTCTGGGCCTGTCTACTTCGTTGTCCGTGAGCTGCTGGTGGCCC
TAGCCCTTTCCCAGGGGTTCAGGTGCAGCCCTGGGGTTGTGTGGGACAGCCAGTG
TGGGCAGTGGAGCTGCTCTGGAGGCCTGCTTTAGGGTGCAGCCTGGGTGTCTTGC
CCGAGTGTAGTGACCCGCCTGCCGTGTCCTCACGGGGTTCCCTAGCAGGAAGGG
CCAAGGGTCCCCTGTGTCTGTGACACGTGAGTGTTTCCCATGGGTCCCATCAAAT
GGGTCCACGGAGGGAGAGCATGAGTGAACTGGTACCCCCCACCCCCACCCTCTT
CCCCACCTGGAGGCATGAGCCCCTGGTTGGACATATGTCCTCCTCCTGTACCCCC
AGATGGCAGCGCTGGAGGAGGACTTGTACTGGGGCCGCTGGCCCCACATCAGTG
TCCGTCATCCCGGCGTCCAGCCCCTGGTGTGCTGAAGCTCAGGCCCTTCCTTGTG
CCCTAGTCTCCTGCACTGAGCTTGGTGAGCCCATTCGGATACTGCTGGATGCATG
GGTCGGGAGGGAGGTGCCCTGGGTTGGGTCGATGATGAGAACCTTATATTGTCCT
GAAGAGAGGTGATGACTTAAAAATCATTCTCAAAAGGATTATGCTGAGGCCCGG
CCTAGGTTAGAATTTTGGAAGAGGATGCTGGGATCCTGAGGTCCCCAGGAGAGC
CCCTTTATTTTTGGCTGGAGACCTGGAGGCCCTGAAGGGTATCTGGGGGGCCCAG
CCCTGTCCCTATACTCCTCTGTGAGGCAGCCCAGGCTCTCTGTGTGGGCTTGGTGT
TGGCACTCTGGTTTTCTGGGGGCGGTTCAGGTTGCTGGTTCCTTCTGAACCATTGT
TCTCCCCTTGATTCTTTCAGTGAGCTCTTCTGCTCAGGCGGGTCCCCTGGCATTGA
CCAGCATAGGTAAGTGGATCCTGCTGGGTTCATGGGCCATGAGCCAGACCACAT
GGAGTGACTGAGGGTCAGCCTTCAGGACAGGAAGATTTCCTCGGGGAGCCAACC
TGAATCCCCACCGTGGAGGGATGTGTTAGTCCTCCAGGAGGGTGGCTTCCTAGGG
AATCTGACCCCAGGAGGACACCTTTGCTGAAGCCCTGCAGGGGTGATGGTGGTC
CAGGCTGGAAAGTTTTCCTTCAGTCTCATGTGGGGCAGCTCCCTGCTGACATTCC
GTGCCTGCGTCTGACCTTGAGGACTGCCCGGGCCACATGAGTGGGGCAACAAGG
GCTCCAGGGTGCCACGGGGATTATGAGTGGGGCTCTCCATCCCCGGAGAGAGGT
GGGCAATCAGGCCCATGTGGTCAGGCTTGTAGGGTTCGGTGGTGGGGAGAGCCA
AGGTTTCCCTGGGTCCATTACCGTCACTGTTTCACCTGGATCCCATCACATGGGTC
CATGGAGGGAGAGCATGTGTGAACAGGCACCCCTTGCTCCCTTTCCCACATGAGC
CCCTGGTTGGACATATGTCCTCCTCCTGTCCCCCCAGATGGTGGCCCTGGAGGAG
GACTTGTATTGGGGCCACTGGCCCCACGTCAGTGTTCGTCAGCCTGGCATCCAGC
CCCTGGTGTGCTGAAGCTCAGGCCCTTCCTGTTGCCCTAGTCTCCTGCACTGAGCT
TGGTGAGTCCATTCGGGGACTGCTGGATGCATGGGGGGTAGGGAGGTGCCCTG
GGTTGGGTCGATGATGAGAACCTTATATTGTCCTGAAGAGAGGTGATGACTTAAA
AATCATTCTCAAAAGGATTATGCTGAGGCCCGGCCTAGGTTAGAATTTTGGAAGA
GGATTCTCGGATCCTGAGATCCCCAGCCAGGCCACTGAATTTTTGGCTGGAGTCC
TTGAGGCCCTGAAGGGTGTCTGAGGGCTGCTGAAACCTGTCTCTGTGCTTGTCCG
TGAGGCAACCCACATTCGCTGTGTGGACTTGGTGTTGGCCTTCCCGTTCTCCGGG
GATGGGACATACTGTCAGCTTGTCCCTGAGTACCCGTTCTCCCCTGTGTCTTTCAG
TGAGCTCTTCTGCCCAGGTGGTCCCCCTGGTCTTGATGGGCATAGGTGAGTACAT
CCTGCTGGTGTCCTGGTCCATGAGCCAGGGTGCGTACGGTTGCCGGAGGTCACCT
TTAGGCCATGTGGGTTTCCTCAGGGAGCTGACCTGAATCCCCACTGGGAGGGATG
GTTTCTCCAGGAGGATGTCTTCCATAGAAACTGGTTCCAAGAAGGATGCCTTCCC
TGAAGCTGTGCAGGGGTGACGATGGTCCAGGCAGGAAAGGGTCCATTCAACCCA
ATGCAGTACAGCTTCTAGCTGAGACTCGGTGCACGGGTGAGGGCTTGAGGACTC
CCAGGCAACATGAGGGGTTCCACAGGATCTCCGGAGGCCACAGGTGTTAAGCAG
GAACTCTCCATCCTTGAGGCTTGCTGGGGGTGGCGGGATGGCAGCAGGGTGTGG
GTGATCGAGTTTATGGATTCAGGCCAAGATTCAAGGGTCAGGCCCCACCCATTGC
CCTGATGGGACCAACCCTGTTGTGCAATGGGCTGTCCCACTGGTGCCTCCTTGAG
CCTGGTGTGGGCAGCGTGGCTTCAATGGGGTTGGGTCAAGGCTGAGCTCTGGCCC
GCACCTGGTGGTTGGCCCTGGGAATTGTGGACTTTGGGCCAAGGGTGCAGCCCG
CCTGCCTCTGGAGGTCA
SEQ ID NO: 5 (hum 2)
TGCTCTGCGTCCATGTTGGGTCAGTGTTCTGTAAGGATTCCATTACAAGGGTATC
CCCTACCCACCTTCCCTACATTGATACATGAGGCCCTGGTTGGACACATGTCCTC
CTCCTGTCCCTACAGATGGTGACCCTGAAGAAAGACTTCAGTTGGGCCTGAAGGC
CCCGGGCCAGTGTCCATCATCCAGGTGCTCAGCTCCCAGTGCACTGAAGCTCAGA
CCCTTCCTGGTTCCCTGGTCTCTTGCACTGAGCTCTGGTGAGCACAACTGGGTGCT
GCTGGATGCATGCATGGGGAGGGGGGTGCCCTGGGTTGGGGTGGTGATGAGAAC
CTTGTATTCTTCTGAAGAGAGGTGATGACTTAAAAACCATGCTCAATAGGATTAC
ACTTAGGCCGAACCTAGGTGAGAATGTTGGAAGAGGACGTTGGGATCCTATTAT
CCCTGGCAGAGCCACTGTATTTTGGGCTGGAGACCTGGAGGCCCTGAAAGGGCA
TCTGGAGGGGGCCCAACCCTGTCTCTGTGCTCCTGCATGAGGGTCCCCAGGCTCC
CTGTCCAGCCTTGGTGTTGGTGCTCTGGTACCCTGGGGATGGAACAAGTTGGTGG
CTCTTTCCTGAGCCCCCATTCTCCCTTTGTGTCTTTCAGTGAGCTTTTCTGCCCAG
GTGGGCACCCTGGCATTGATTAGCCTCCTTGGTTGTCCATGACCTGGCTGGTGGC
CCGATCCTGTGCCCAGTGGTCCCAGGGCAGCCCTGGCATCATACGGGGCAGCTGT
TTTGGGCAGTGGAGGTGCTCTCCAGGCCTGAAGTATGGCACAGCCTGGGCATTTT
ACCTGAGACCAGTGACCTGCCTGCCTTGTCCTTCCTGGGTTCGGTGGCAGGGAGG
GCCAAGGGTGCCCTGCGTCAGTGATGAGTCAGTATTTTGTGTGGATCTCATCACC
TGTCCATGGAGGGAAAGCAAGCATGAGCTGGCACCCAACACCCCCTTCTCCGCA
TGGAGGGTGGTCACATGTCCTCCACCTGTGCCCCCAGATGGTGAGACTTGAGGA
AGACTTCCACTGGGCCTGCTTTTCCTGAGCCAGTGCCCATCACCCAGGTGCTCAG
TCCCTGGTGCACTGAAGCTCCAGCCCTTCCTGGCGCCCTGGTCTCCTGAACTGAG
CTGTGGTGAGCACATCCGGGTTTCACTGGATGTTTGTGCGGGGAGGGGGTTCTCT
GGGTTGGGTCAATGATGAGAACCTTATATTGTCCTGAAGAGAGGTGATGACTTAA
AAATCATGCTCAATAGGATTACGCTGAGGCCCAGCCTAGGGGAGAATTTTGGCA
GAGGATGCCGGGATCCCAAGATTTCCGGGAGGGCCACTTGTATTTTGGGTTGGA
GACCTGGAGGCCCTGAAGGGCGTCTGGAGGTGGCCCAGTGCTTTCTTTGCGCTCC
TCTGTGAGGCAGCCCAGGCTCCCTTTGTGGGCTTGGTGTTTGCGCTCCTCTTCTCC
AGTGATGGGGCACGTTGGGGGCTCCTCCCTGAGTCCTGGTTCTCCCCTTGTGTCTT
TCAGTGAGCTCTTCTGCCAAGCGGTTCCCCTGGCATTGACCAACATAGGTGAGTG
GATCCTGCTGGCATCATGGGCTATGAGCTAGGCCGCATGGGTTTGCTGAGGGTCA
GACTTCAGGCCCCAAGGGTTTCCTCAGGGAACCGACCTCAATTCCCACCATGTGA
CTGCCAGGTTTGCAACAGTGCATAGCTTCCGAGCTACCCTCCTTGGTTGGCCGGG
AGCTGGTTGGAGGCTGTGGCCCATGCCTAGTAGTCACAGGGCAGGCAGACTGCC
CTGTTTTTTCAGGGTGTGGTGACGGGGAGGGCCAAGGGTGCCCTGCATCCATGAT
GGGTCAGGGATTTCAGGAGGGATGGTTTCTCCAGGAGGGCAGCTTCCTTGGGAA
TCTTACCCAAGGAGGATGTCTTCCCTGAAGCCCTGCAGGGGTGACGATGGTCCAG
ATAGCAAAGGTTTCCTTCAGCCCCATGCAGGACATCACCTAGTTGAGATATGGTA
TGTGGGTCAGGCCTTGAAGACTGCCCAGGCCACCTGAGGGGCCCAGAAGGGCTC
TGGGGTGCCACAGGTGTTATGTAGGAGCTCTCAGTCCCCAGACTTGTTTCGTGGG
GTTGGTGACGGGGTTGGGTTGTGGAGGTTCAAGGATTTCATCCCACCTGTGACCC
TGAATGGGGCCAACCCTGTTGAGCAACAGGCCATCCCAATGGCACGTATTGAGC
CTGGTGGGGTGTGGTGCAGTTTCAATAGGGTTGAGTCAAGGCTGAGATCCATCTG
GTGCTTGGTGCTGCTGCTGGCCAGCCCTGGGACCCGTGGGCTTTGGGCTAAGGGG
GTAGCCCACATCCTATTGAGGGTAACCCATTCTCCGAGTTTGAGTAACGGTTAAC
AGTGGCATTCTGAGGTGGGAAGGCCCACTGTCTAGGGTCCTATACTGGGAATTTG
CCACCCCGGGTTCTTCTGAGACAAGTGGTCACCTCCAGGGTCATGGGGTCTTGGG
CCCAAGGAAAGGAGAAGATACCATGTGGCCCTTGATTGGCTACATGTGGCCCAG
GTCTGTTTGGGAGGTGTGTGGGATGTGGATGGGGTCCCACCAATGAAAGCACAT
GGCCTGGGTCCCCAGGCTGTGATTTTCAGTGCACACTTGGAGAGGGTGCCTCGGC
GGGTACCTACCACATCCATGGGCAGGCTATGTGACTACCAGGTTTGCAACAGTGC
ATAGCTTCCAAGCTACCCTCCTTGGTTGGTCGGGAGCTGGTTGGAGGCTGTGGCC
CATGCCTAGTGGTCACAGGGCAGGCAGACGGCCCTGTTTTTTCAGGGTATGGTGA
TGGGGAGGGCCAAGCATCCATGATGAGTCGGTGTTCCACATGGAACCCATTACG
TGGGTATCTCTTACCCCTTCCTCATATGGAGATGTGAGGCCCTATTTGGACACAT
ATCCTCCTCCTGTACCCCCAGATGGTGAGCCTGGAGGAAGACTTGCGTTGGGCCC
AAAGGCCCTAGACCAGTGTCTGTCAGCCAGGTTCCCAACCCCCGATGCGCTGAA
GCTCAAGCCTTTCCTGGCACCCTGGTCTCCTGCACTGAGCTGTGGTGAGTATATC
CTGGTCCTGCTGGATGCATGTGCAGGGAGGTGGGTGCCTTGGGTTGGGTCAATGA
TGAGAACCTTATATTGTTCTGAAGAGAGGTGATTATTTAAAAATCATGCTCAATA
GGATTACGCTGAGGCCCAGCCTAGTTGAGAATTTTGGAGGAGGACACTGG
SEQ ID NO: 6 (hum 3)
AGGCCCAGCCTAGGTGATAATTTTGGAAGAGGACACTGGGTCCCGAAGCCCCCT
GCAGGGCCCCTGTGTTTTGGGCTGGAGACCTTGAGGCCCTGAAAGGCATCTGGTG
GACCCAACTCTATCTCTGCCCTCCTCAATGAGGCAGCCCAGCCTCCCTGTACTGG
CTTGGTATCCGGGCTCCGGGTCTCTGGGGACGGGGCACATTGGAGGCTCCTTCCT
GAGCCCCCATTCTCTCCTGTGTCTTTCAGTGAGCTCTTCGGCCCAAGTGGTCCCCC
CGGCCTTAAGCGGCATAGGTGAGTGCATCCTGTTTTGATCATGGGCCATGAGTGT
TGCAGCATGGGGTCCCCGACATTCAGCCTTCAGGCCACTAGGCTTTTCTCAGGCT
GCAAACCTACTCTCCACCAGGGAGCGATGGTTTCTCAGGAGGGCCACTTCCTTCG
GAATCTGACCCATGGAGCACGCCTTTCCTGAAGCCCTGCAGAGGTGACAGTGGT
CCAGGCAGGAAGGGGTTCCTTCAGCCCCAATGTAGGGCAGCTCCCAGCTGAAAC
TTGGTGGACAGATCAGGCCTGGAGTACTGCCCAGGGCATATGGGGAGGAGGGAC
ACAGGGAATGTGGCGCACCCTAGAGGTCACACTGGAGTTCTGTCTCCCTGGAGA
GGGGTGAGTGATTGGGCCCACATGGTCAGGCTGTGGAGATTCAAGGAATGCTCC
ACACCGATGGCCTTGAATGGGACCAACCCTATTGAGCAATGGGCCATCTTAGTG
GCACCTCCTTGAACCTGGTGGGGGATGGCACTCTTTCAATGAGGTTGGGTCAAGG
CTGAGCTCTGGCTGGCACCCAGTGGTGCTGCAGGCTGGCCCTGGGACTTGTGGGC
TTTGGGCCAAGGGGGAAGCTTCCATCCTGCTGGGAACAACCCTTCCTTGAGGTCC
AGTGGCAGGAAGGGGGGCTTCCTGAGGTGGAAAGCCCTACTGTTGAGGGACCTG
TCCTGGGAATTTGCTGTCCAAGCTGCTTCTGAGCCATGGGGTCACCTCTAGGGTG
GTGGGGTCTTGAGCCCAAGGAAAGGAGAGGACACCATGTGGTTCCTGATTGGCT
GAGTGTGGCCCGGCTCTGTGTGTCGGAGTTGTGCAGGGTATGGATTGGGACCCAC
CAGTGAAAGCACATGGCCCTGGAGGTCCGCAGGCTTGGAGAGGGCACCAGATCT
GGCACCCACCATGCCCAGGTACAGGCTCTGGGACTGCCAGGTTGCAGCAGCTCTT
GGCATCTGGGCCACCCTCCTTGGTTGGCCAGGAGCTAGTTGGTGGCCCTGTCCCA
TGCCCCATGTCCCTGGTATGGCCATGGGGTCATGCAGAGGAACCGGCCCTGGCAT
GGAGGTGCTCTTGCAACATGCAGTAGGGTGCATCCCAGGCATCTTGCCTGAGCCA
GGTGACCCGCTTGCCCTGTCTCTCCAGGGTTTGGTGGCAGGGAGGGCCAAGGGT
GCCCCGTGTCCATGTTGGGTCAGTGTTCAGCATGGATCCCATTGCATGAGTATCA
CTTAGCTCCTTCCCAGCATTGATATGTGAGGCCCTGGTTGGACACTTGTCCTCCTG
TCCCTCCAGATGGTGAGCCTGGAGGAAGACTCGTGTTGGGCCCACAGGCCCCAG
GCCAGTGTACGTCAGCCAGGTGCTCAGCCCATGGTGTGCTGAAGCTCTGACCCTT
CCTGACTCCGTGGTCTCCTGCACTGAGCTGTGATGACCATATCCAGGTCCTGCTA
GATGCATGCGTGGGGAGGCAGGGTGCCCTGGGTTGGGTCAATGATGAGAACCTT
GTATTATCTTGAAGAGAGGTGATGACTTAAAAATCATGCTCAATAGGATTACACT
GAGGCCCAGACTAGGTGAGAATTTTGGAAGAGGATGCTGGGATCCTGAGGTCCC
CAACAGGGCCACCATATTTTGGGCTGGAGACCTGGAGGTCCTGAAGGGCATCTG
TAGGGGGCCCAACCCTGTCTCTGCACTCCTCCCTGAGGCAGCCCATGCTCCCTAT
TCGGGATCGGTATTGGTGCTCCACTTCCCTGGGGGCAGGACACGTGGGTGGCTAC
TCCCTGAGCCCCCGTTCTCCCCTTGTGTCTTTCAGTGAGCTCTTCCAAACAGGTGG
GCTCCCTAGCATTGACCGACAGAGGTGAGTGGATCCTTCTGGGATTATGGGCCAT
GAGCCAGGCCATGTGGGGTCCCCAAGGTTCAGCCTTTGGATCCCGAGAGTTGTCT
CAGACAGCCAACCTGATTTGCCACCTGGGAGAGATGGCTTCTCCAGGAGGGTGG
GTTTTTGGGAATTGGACCCAAGGAGGACGCCTTCCCTGAAGCCCTACTGGGATGA
TGGTGGTCCAGGCAGGAAAGGGTTCCTTCACCCAGCTGAGACTCGGTGTTCCAGT
AAGGCCTCAAGGACCTCCCTGCCACATGAAGGGGCCCAGAGGGGCTACGAGGCA
CCACAGGAGTGACAGGGGAGCTCTTGGTCCCCAGAGTGTGGTACATGAATGGGC
CCATGGGGTTGAGTCGCAGAGATTCAAGGATGCTGCACCACCCATGGCCCTGAA
TGTGACTCTCCTCCTGGAGCCTGGTGGGGGGTGGCGTTTTTTCCCTGGGGTTCGG
TCAAGGTGGAACTCTGGTCAGTACCTGGTGGTAATGTAGGTCAGCCCTGGGACTC
GTGGGCTTTGGGCCAACGGACAGCCCACATCCCTCTGGGGGTCACCCTTTCTCTG
AGGTCGAGTGTCGTGAAGGGTGGCCTCCTGAGGTGGGAAAGCCCAATGTCGAAG
GACCTGTACTGAGAATTTGTTGCTTAGGCTGCTTAGGATCCATGTTGTCACCTCC
AGGGTGGTGGGATCTTGGGACCAAGCAAAGGAGAGGGCACCATGTGGCTGCCAG
TGGCTATGTGTGACTGGCTCTGTGTCTGAGAGGTGTGTGGATGGGGACCCACCAG
AGAAAGCAAGTGGCCCTGCAGCCCCAAGATGTGATTTCTGGTGCAGGCTTGGAG
AGGGTGCCTATACCTGCTCCCACCACGCCCAGGGGCAGGCTTTGCCACTGCAGGT
CTGCAGTGCCACGTGGCA
SEQ ID NO: 7 (hum 4)
GGTTACCCATCAGGAGGGATGGTTCCTCCAGGAGGGTCGCTTCCTTGGGAATCTG
ACTCTAGGAGCATGCATTTCCTGAAGCTCTGCAGGGTGATGGTGGTCCAGGCGG
GAAAGGGTTCCTTCAGCCCAACATAATGCAGTTCCCAGCTGAAACTCGGTGGCA
GATCAGGCTCAAGGACTGCTCGGGCCACATGATGGGGGCCACAGGGGATTTGGG
GTGCCCTGGGGGTCATGCTGGAGTTCTTGCCCCAAGAGGGGTGGGTGATAGGGC
CCATGCGATCAGGCTGCGGAGATTCAAGGAATGCTACCCACCCATGGCCCTGAA
TGGGACTGACCCTGTTGAACAATGGGCCATTTTAATGGTGCCTCCTTGAGCCTGG
TAGGTAGTGGCATTGTTTCAATGCGGTTGGGTCAAGGCTGAACTCTGGCTGGTGC
CCGGTGGTGCCCACATGGGCTTTGGGCCAAGGGGGCAGCTCATATCCCTCTGGG
GTCACCCCTTCCTTGAGATTCAGTGGCAGGAAGGGTGTTCTCCTGAGGTGGAAGA
CCTGCTGTCAAGGGACTTGTCCTGGGAATTTGCCACCTAGGCTGCTTCTGAGCCA
TGGAGTCACCTCCAGGGTGGCAGGGTCTTGAGCCCAGGGAAAGGAGACACCATA
TGGTTTCTGACTGGGAGTGTGGCCTGGATCTGTGTGTGGGAGGTGTGCAGGGCAT
GGATGGGGACCCATCAGAGAAAGCATGTGGCCCTGTAGGCCCCCAGGCTTTGAT
TTCTGGTGTGTGCTTGCAGTGGGCACCAGGGCAGCCCTACCATGCCCAGGGGCA
GGCTCTGTGACTGCCAGGTCTGGAGTGGCTCCTGGCATGAAGACCACGCTCCTTG
GTTGGCCATGAGCAGGTTGGTTGCCCTAGCCTGTGCCCTGTGGTCCTGGTGCAGC
CCTGGGGTTGTTTGGGGAGACAGCACCGGCAGTGGAGGTGCTCTCAAGGCCTGC
AGTAGGGCGCAGCCCAGGTGTCTTGCCCGAGCCCAGTAACCCACTTGCCCTGTCT
TTCCAGGGTTTGGTGGCAGGGAGGGCCAACTGTGCCCCGCATCTGAAATGGATC
AGTGTTCTGCATGGATCCCATCACAAGGGTATCCCCTACCCAACTTCTGTGCATT
GTTATGTGAGGCCCTGGTTGGACACATGTCCTCCTCCTGTCCCTGCAGATGCTGA
CCCTGGAGGAAGACTTGTGTCGGGCCCCCAGGCCCCAGGCCAGTGTCCATCAGC
CAGGTGCTCAGCTCCCAGTGCGCTGAAGCTCAGACCCTTCCTGGCACCCTGGTGT
CCAGCACTGAGCTGTGGTGAGCCCATCCGGGTCCTGCTGGATGCATGAATGGGG
AGGGGCCTGCCCAGAGTTGGGTCAATGATGAGAACCTTACATTGTCCTGAAGAG
AGATGATGACTTAAAAATCATGCTCAATAGGATTACGCTGAGGCCCAGCCTAGG
TGAGAATTTTGGAAGAGGACGGCTGGGATCCTGAGATCCCAGTCAGGGTCACTG
TATTTTGGGCTGGAGACCTGGAGGCCCTAAAGGGCATCTGGAAGGGGGCCCAGC
CCTGTCTCTGTGCTCCTCCGTGAGGCAACCCACACTCCCTGTGTGGGCTTGGTGTT
GGTGCTCTGGTTCCCCAGGGACAGGGCGGGTTGGTGGCTCCTTCCTGAGCCCCTA
TTCTCCCTTTGTGTCTTTCAGTGAGCTTTTCCACCCAGGTGGGCCCCCTGGCATTG
ACCGACATAGGTGAGTGCATCCTACTGGGATAATGGGCCACTAGATAGTCTGTGT
GGGGTTGCTGAGGTTTAGCCTTTGGGCCACAAGGGTTTTCTCAGGCAGCCAACCT
GAATTCCCACCTGGGAGAGATGGGTTCTCCAGGAGGCAGCTTTTTTGGGAATCAG
ACCCAAGGAGGACACCTTCCCTGAAGCCCTGCAGGGGTGATGGTGGTCCAGGCA
GGAAAGGGTTCCTTCTGCCCAACTCGAGGCCCAATGCAGGGCAGTGCCCCCCTG
AGAATTGGTGCTCTGGTAAGGGCTCGAGGACTGCCCAGGCCACATGAAGGAGGT
CAGAGGGGCTATGGGGAACCATGGGGGCAACAGGGGAGCTCTTGGCCCCCGGA
GTGTGGTACATGAATGGGCCCATGGGGTTGGGTCACGGAGATTCAAGGATTGCA
TCTCACCCACGGCCCTGAATGCGACCATCCCTGTTGAGCAATGGGCCATCCCAGT
GGGTCTCCTGGGGCCTGGTGTGGGGTGGCATTGTTTTCCTGGGGTTGGGTCAAGG
CGGAGCTCTGACTGGTACCTGATGGTGCTGCAGGTCAGCCCTGGGACTCGTGGGC
TTTGGGCCAAGAGGCAGCCCGCATCCTGCTGGGGGTCGCCTTTTCTCCGAGGTTG
AGTGTCGTGAAGGATGGCCTCCTGAGGTGGGAAGGCCCACTGTCAAGGCACCTA
GACTAGGAATTGTTCCTCAGGCTGCTTGGGAGCCATGGTGTCACCTCCAGGGTGG
CGGGATCTTGGGACTAAGCAAAGGAGGGGACACTACGTGGCTCCCAATTGGCCA
TGTGTGGCTGGCTCTGTGCCTGGGAAGTGTGTGGAGTATGGATGGGGACCCACC
AGAGAAAGCAAGTGGCCCTGCAGTCCCAGAATGTAATTTCTGGTGTAGGCTTGG
AGAGGGTGCCTATATGCATGCCCACCATGCCCAGGAGCAGGCTATGCCACTTCA
G
SEQ ID NO: 8 (hum 5)
GTTCAATAGGATTATGTGGAGACTTAACATAAGTGACAAACCTAAGACAAGAAG
TTAGAAATCCTTGTTTCTTTTAGGTGAATAATTTTGGAGCTTGAGAAAAAGACAC
CATTGTGGCGAACTTGCTTTGCCAAACCCTGTGTATGCATTTTTGGGTAAACAAC
ACATTTTCATCCTTTAAGCATTGTTTCCATTATTTATAATTCCTAATAGTATGAGA
CATTTAGGACTCATTCCTTGGTTCTTCTTCTCTCATTGTATCTTTCATTGTATCTTT
CAGTGCTGCAAGAGATGCAATCATCCCATTAATGAAAGCAGAGTGACTGCCTTC
GTCACACATACATTCAAAATAGATCACTAGGCTAGTGATGATTCTCTCTTAAGGC
TATTATGTTTACTCATTTAAAAAATCTGGATCTCAGTATAGATTTTCTTCTCAGTG
AGTGACTGACCTATCTGATATGATGCTATGACAAAATCTTTACTTTTTACCTGAAT
TTGGTGGGATTGAAAGTTGAGAACATATTAATTTATTTTCTGTTTCACTTGATTAA
TAATGCCCAATCTAACATGCAGTGTTCACTTGGGTACATATATACAACTACATCA
AATTTTTTCTTGTTTTACCTTAAAAGTGTTAAGATATTGTAATGGTATGGCCAGTT
TACTCACTGGTAAACTTTTCATAACAGAAATATCATGCCAGTGGACACAAATATG
TAACATCAGTAATTTGCTTTCCTCATGAAACAGAGAACTGAATAAGTTGATGGGT
AAACTAGTATCTGAATTATAAACCCCAAATATCATGTGACTTCATCATTTTCAGT
ATAATGAAAATACAGGCTGTGAATGCTTCCAGTTGGTCCTGAAAGGGCCTTTGAG
GTGAGTAGGTTCACCAAAGCGGCAACATATGTGTCCCAGTGGGCATTCTACTCCT
CAGTAAGTGACTTGACATTCTTTATGGCTTCCTCATGATTAAAACAAGTCTTTTTA
TTGAAATTTTACTGAAATAATGATGAAACAATGTAATATTCTTTTCATCTACATAT
AAGAGTAGACATAGATTAACCTCTACCTTTTTTTTTTTTTTTTTTGAGACGGAGTC
TCGCTCTGTTGCCCAGGCTAGAGTACAGTGGTGCGATCTCAGCTCATTGCAACCT
CTGCCTCCCAGGTTCAAGCGATTCTCCTGCCTCAGCCTCCTGAGTAGCTGGGATT
ACAGGCACGTGCTACCATTCCTGGCTAATTTTTTTGTAATTTTAGTAGAGACATG
GTTTCACCATGTTGGTCAGGCTGTTCTTGAACTCCTGACCTTGTGATCTGCCCACC
TCTGCCTCCCAAAGTGCTGGGATTACAGGCTTGAGCCCCCGTGCCTGGCCTTCAC
CTCTACCTATTATAATATTTTCATGTTATTCCATTTTGTCATGTTTGTGGGTCATTT
GATGAGGGTTCACAATATGAAAACTGGGCATTTTGGCCAAGATGCATGTAAATG
AATACAACTCCATGTGCCCTGATAACTGATTTCTAGATCCTGAGATGCAAGTGGC
TTCCAGAGTGTCTGTATTTTATGAAAGTTCATATACAGTAAACCATGCTAATTGTT
ACTGATACAGCTTCCTTGAATATATAAAGCGATGATTAATGGGATGTTTTGTTTCT
GCAGAAAACCACTGAATTGCTTCTCCAAAGCCAAGTGACCTGTATGATTTGTCCT
TATTATATTTTGTGAACCAGTGGACTAAAACCTTTATAATGGGTCATTTTTTTAGT
ATTGTCAATATTGCTCAATTAACAAACATGACTCATATACACAGGTGAGATGTAG
TTCTCCCTTTGACATTTGCTCTACATCTGTCCCTTAGATGATGATATGGAAGAAAA
GCACTCTTTGGCCTGTTGTGACTGGGACAGTTGACAGCACCCAGGTGTCCTTTAA
TGAAAATGCTCTTGACACCAATGCATCCTAGCATCACAGCTTCAGGAAGCCTTCT
CAAGTGTGCATGGGGAGTACTATGTCTTTCATCAATAATGAAATCTTCTGATTTG
GTGAGAAATAATGCCTTAAAATTACACTCAATAGGATTATGCTGAGGCTCAGCCT
ACCTAGATGAAAATGCTGAAACAGATAATAGTGACTTCTTATTTCCCTTCAAGGG
GAATTTTATTTTGAACTTGAAATAACAAAATCTTTATGGGTAACTTGTTTGCCCAA
CTGTATCAGTGCACCTATAAATGAACAAAAAATTCTCAGCCTATAAGCTTTGCTT
CAGTGACCTTTAATTTCTAATAGTAAGGTATATTCAGGTCTCATTCTTTGGCTTTT
TCTCTTATTGTGTATTTCAGTAAGACATGCTGCCAAGAGATGTGCCATTCTATTAT
AAAAGATCAGTAGCTTCCTTTACCGACGTGTATATTCTATCTAGAACATTGAGCT
ATGGAAGACTCCCACCTAAGGGAATTAGTTTTACACCTTCAGGTAATCTGAACTT
CAATATGAGTTTTGCTTTATGATATAATCCTTATGACAAAATCTTTACTTTTTATC
TGAACTTCATACAATCAATATTGAAGATTTTTTAAATTCACTGTTACCTAGTCCAA
TATTTAATATACATTTGGCTGTAGATACATGACTAGACAAAAATTTTTTATATTAT
TATTGTGGGGGCTCTAACAGTGTTTCACTGTTTTGAGTTACTGATGAACTTTCTGT
AACTGTGAGACCATGGTAATGGAAGAGACCGTGGTAATGGGGAGGAGAATGTGA
GGATCAGTATGTACTTTGCCTATAAAACTTAAATATTTAGTAATTTGATCACCAA
ATTGATACTCAAATTTCAGTTCCTATCATGTGTAAACCAAAAAGTATCTGAGATT
TAGAAGTTTATTTAGAAGTGGATTTTGCCAAGGTTAAGCACATGCTCAGAAGAAA
TAAACCCAAAATCACAGAAACCGTCTGGGGTCTATGTCTTTCTCCAAAGATGATT
TTGAGGGCTTCAGTGTTTAAAGGGGAAAAGTGGGCTGGAGGGGG
SEQ ID NO: 9 (hum 6)
TGAGAAGAGCACCCTCTGGTGTTGTCACAGAGGCAAGTGCTACCGCACAGGCAT
GCTGCAGTGAATTTAACTGATCCTCTGTCCCTGCAACCGTTGTTTAAGGATGCTA
TTCTGGTAAGGGTTTACAAGACAACGTAAATATATGTATAAAGAGTATCTTCAGG
AGACGTAATAATGTAAAAATCATGCTCAATAGAATTAAGCTGAGGCTCAAAATA
GTGCCAATCCTTGCAAGAAGGTGTGAGAAGTCATTAGATTTGGGGGGTATGGGTT
ATTCCATTTTGAACTTGGTAGGAAGAAACCCTTGGGGGAATCTTACATCATTTGC
CTCTACCTACTTCTAAAGGAACTTTTAAAATTCTTTTATCTTGTCTTTATTCTTAAT
ACTCATACATTATTAGAGAGAATATCTTGGTTTATTTTTTCCTCATGGTGTGCTTC
AGAATGTTCATCCAGCAAGAGGTGTGTCATTACACGGATTATATATATGAGTTCT
GTCTTTCGTAATTAAAAAATTTCAAGTGGACCATTTGAATATTAAACATTGGCCT
TCAAAATGATTACATTTACCTGTTCATACAAAATTAGTGAGCAATGATCTATTCTT
TCCAATGAGTGACTGATCTAATGAGGTCCTCATTAAAACTCCTTTTTTTTTTTTTTT
TTTTTTTGCCTGAACGTACTTTGATTTAAAGATGTGACATTTAGTAATTCATTGCA
ATCAACTCTTTAGTGATTGCACAAATAGACAATCCTTTTCTATTATTTTATTTTTT
GGTTGCTGTGTATGGTGTTATGATGTTTGTGGATCATTGAGGCACTACTCACAAC
TGAAAGACTTTGGCAATTTTAGGGAATGGCTTGAGATTCAGCTATAGGTTACCCT
TGATTGAAATTGAATACAATACAGGTAGTCTGAAATCTCATTTCCTTATCCTGAG
TCTCACTCAGGTGTCATTAGATTCATTATAAAGAAATGCATACACTATGTATGAT
GCCAGTTGTTACTGATATGAAGACGATAAGAAGCTTTTATTTCTGCAGGGAAATA
ATCCCTTACCAGGTCATCAAAAGACATGATACCTATGTGCTTGGCCCTTACTGTA
CACGGGGAATCAGTGGTCTACCACAGCTTAAGTAACGGGTCATATTTGGAGTATC
ACACATCTCAGTCTTGTAGAAATTAGGAACAGCAATTAGGAGTCATGCACATAT
AAGAGATGTAATCCCACCCTTTGACTATAGCCTACTCTTGTCTTTTACAGAAAAG
ACTGTGGAGGAAGAAAACCCTTTACCCTGTTGTTCAGGGAGAAACTGACACCAC
TCAACTGCCTGGCACTGAAAATGTGGCATCCAGTCCACTTTACCATCAGTGTTTA
AGGAAACCATCTCTGGTAAGCATATTTGATCCAAGTGTGCATATGCACATGAGTG
CCATGGGCTGGGTCAATGATGAGATGTTACCTTGAAGAGAAATGATGACGTAAA
AATTAAGTTCAGTTGGATTACGCTGAGGCCCAGCCTAGGTAACAATTGTGAGAC
AAGAAGTTTGGATTTCTTAGTTTCCTTCCATTGGGGACTTTATTTTGGGATTCTGG
CAATGGAATCTTATTGCGAAACTTGTTTTGCACGCTTCTATCTGTGTGCTTCTAAG
TAAACTATAAATTCTGATTCTTTAAGTGCTATTTAAGTTTCCTACAATTCCTAATT
TTCTTGGATATTTGGCACTAAGACCCTTGCTCTTTTTCTCTCATTGGATGATTTAG
TAAGTCCTACTGGCACTATTCTATTGAGTGGAGGTGAGAGGCT
SEQ ID NO: 10 (hum 7)
TGTTTGCCCATGAAACTTGAAATTAGATAAATTGATGAGCAATCTGGTGCCTGAC
TTTTCTTTCTCAAGTACCTTTGGCTTCGTTTTCATTATGATGAAAGTGTGTGCTGT
GAATAATGCCAATGGTAAATAAGACCTTTATGGACACTTATATGAATGACAGTCA
GGGCCGTTCCTTCAGGCCACATGATTATCGTTATGCATTCTTCTTCTCAATGAGTG
ACTAGCATACTTGCCAGGATCTACAAGAGGGAAACTTTATTTTTTACTAAAATTG
CATCGATATAATGTTGACAAGTTTTGTGATTATTGGTATCATCTCTAAGATGTGAT
GCTCATTTGGTCTAAATATAGAAGTTAACATATTAACTTCTAATTTATATATGTCA
GGTTGCTATGTTTTTACAATGTTTGTGGTTTATTGATGATGACTCACAACATAAAG
ACTATGGCATTATGGCTACATGTTCTAATTCTGATATATACATATCCCATCATTCA
TTAAATTAAATACAATTATGAGTAGTATGATGTCTGTTTTCTAAAATCTGAGTTTC
ACCTGGCTCCAGTATGATCATTATATGATGGAGTGCAAGCAACATGTTAATGCCA
ATTGTTATTGATATGGCTTCCTTGAATATGTAAACAAGACTGATCAATTGAAGGT
CTTATTTCTGCATTAAACTACCTGAGATATTCTCCAAATTCAAGAGACCTGTATA
GTTAGTCTTTATTCTGTTTGATGAATAAATGGCCTATAGCCTATCTAATGGATCAG
TTTTTCCAGTACTAATCATATCTCTTCATTAAGAAATAGCATGCATAAGCCATAC
ATACAGGAGGTAATATTTGTCATCTTGATCTACTGTCTTATCCTCCTCCCCTTAGA
TAAGGATGACTGAGGAAGAGTACTCTTTGGCTTGTTGACACCAGCACAGCTGAC
ACACCCAGATATCTGTTTGGTCTCCTGTGAACTTTCAACCAGGATTTAAGGATGC
CACTCTGGTAGGTATTTTCAGACAAAGTGAATATGTGATTGTAGAGTTCCATGGC
TTGTTTCAACAATAAGACCTTATGTTGTCTTGAGGACATGAGATGACATGAAAAC
TAGACTGGCTGGTCCTTATCTTTTCTGGGGTATTCTAGTTTGAGCTTAAGATAAAG
AAATATATTTGGGATATAGTTTCTCTCACCTGTTTCTATTAGCTTCTAGAAGACCA
CTTATCAATTCTTTCCTCCTGATTTAGTGGAAAACATTTTGGCAGTTCCTCAAAAA
GTTAAAGATAAAATTACCATGTAACCCAACAATTCTGCTCCTAAGTACACACTCA
AAAGAATTGAATACAAATGTTCAAATGAATACATACACATGAATGTTCATAGCA
GCACTATTCACTGGCTGGAGTTCCAGACCAGCCTGGCCAACGTGGCAAGACCCT
GTCTCTACTAAAAATAGAAAAATTAGCTGGGCATGGTTGCTCACGCTTGTAGTCT
CAGCTACTCAGGAGGCTGAGGCACAAGAATCACTTGAACCCAGGAGGCAGAGGC
TGCAGTGAGCTGAGATCACACCATTGCACTCCAGCATGGGTGACAAGTGAGATT
CTGTCTCAAAAAAAAAAAAGTGAAGGTATGATGACAATGTCACACAAAATAGTG
AATACCAATAAAAAGATAGAAATTATTAAAAAGAACCAAATGAAAATTCTGGAG
TTGAAAAGTACAATAACTAAAATTTAAAAATTTATTAGAAAGGCTCAATAGTAG
ATTTGAACTGGGAGAAGAAACAAGAAGTAAACTTGAAGATAGAACAATAGAGA
TTATGTTCAAAAGAACAGAAAGCATAAAGAATAAAGAAACAAAGAGAGTCTCA
GAGAATGTGGAACATCATTAAGCATACCAGCATACACATATGGGAGTACCAGAC
AGGCAGGGGAGATACAA
SEQ ID NO: 11 (rat 0)
ACTACTGTGCTCTTAGAGATCCACTGAGAACAGTCAAACAACTCTGAAGCAGAG
TAATGGGACCCAAAATGAAAACTAATGACAATTTTTTTAAGAGTTAATAATCAAT
TATTTGAAAGATTTTAATTTGACTAACAAGATTTCATAAGGTACGTGTATATCTG
GTTATTCAAAAGCTATAATTGAGAATTGAGACTGGAGTGGAGAGCATTTAGCTA
GTCACATGAGAGATTATCTTAGAGTCTCATTTCCATCCTCCTTACACTCTGCTCAC
CATATGGTTCCAAAAAGAATTCATATTTTGATGTAGATCCATAAGGCATAGTTAA
GAATTCCCTAATCTTGCACAGTTTGATGAGTAGAACACAGTGTCCAACAAACCAA
TTACTTTTTCTCATTTTCCTTTAATATGGATTCTTTGTGTAACTCTCAAATGATAGT
AGTTCCATAGATCTTGTCCTTAGTCTTCTCTCTTCATTTCATCACTGTTTCCTGAAA
GTATTCTAGGGAAACTGTAAGGATGATTGCCATGCTAGGGCCACTAATTTTCACC
TTCAGCGAAGAATTCTGTAGACTTACCTTTTATTGATTTGGGACAAGTTCCCTTGA
ATAATCTGACACTATAAAAACAATGTGATTCAGCATACTACAACTCCCCTGCCTA
ATCTGTCCCATTGAAAACCCAATAGCACCAATATATCCCTCCATCATTCATATCA
AATATGTGAAATCTGTACTCTTGAGCCTTCTTTATCCCATACACTATCCCTTCTCT
TTTGTCATGCTGCATCTTTTGGGAATTATAGCAACAATCTCATAAATACAAAGAA
TACACAAATCCAGTTCCTATGGTAATACAAGTATGATTTATGTCTTTGCCTCTTAA
GATATGACAAACAAAATCTGAAGTCTACAAGGCCCACCATGATCTCTACAGTTCC
CCAATCACATTCAGCTGCCTATCAAAACCCAATACTCCACCTGTTAAAATGCTCG
TCTTTTCTGCCTGGTAACCATCTAATTTTTTACAGTTTGGGGATCTGAATTCTCAG
TCTAAACTGAGCATTTATACAATCCTGCTATAAACTCCTTGTGCTTTATATACATA
CATACATGTCCCAATTTGAGCTTCTCTGTAAAACACCTTAATTGAACAAAACAGC
TCTGAATTGATCTGAAACATCTGCAACTTGCAAAGCAGCAAACACACAATAGTA
CTTGAAAAAGTGTTAATGGAAATTTCAAACTTTCCTTAAGACAAATGTAAGTTGT
AAAATCTTTACTACAGGGGCTGGGGATTTAGCTCAGTGGTAGAGCGCTTACCTAA
GAAGCGCAAGGCCCTGGGTTCGGTCCCCAGCTCTGAAAAAAGAACCAAAAAAAA
AAAAAAAAAATCTTTACTACAGAAATCTTTCATCATAGAATTATGTCACAGATAT
TATTATTCCTTATTTCTTAACTCCTTTGCATAGGTCTGCTTTGTTGAATTATGCAGA
TTTGAAGTGAGTGAAGTACTGGTGGGGTACTGAACATGTCTTTTATTTTAAACAC
AGTCTTTAGGTCTAGTGATGAAACCTCAAGCTTTGAGATGTTTCTGTTCATATTCA
GGAAACTGTTTTGTTTTTTTGTTTTGTTTTTTTTTTTTTAAGTTTTACAAGTACTAA
ACAGCACAAATAGTCCATAGCAGCGAAACATTAAAATTACCAATTCTTAATTGA
AGACAAAAGTCTTCCTCCTGCTCAGGCCCTCCTATAGCAACTCAAAACACCCTCC
CTACCTATTTTAACAGGTACTTAGGGAACTTCTACACTTCAGGAAATATTGTCTTA
CTCATCGAGTTGTTAGAATTATGACAAGCAGTAGTGGGAAATGGTATATTAATAG
CTTTATTTCAAGAATTTACCTTATGGGCAAACTCAACTTGCTTGGCTGTTAGTTTC
CAATGCTTCCAATACCATGAAAGCATGTTTAATGGATGACACTCAGCTTCCAAGT
ACATGGTTTACTAAGGAAGAAAGCAGTTGATGGCTAGTTTCTTATAATAGTGATA
TATGTATGGCTCATGAAAAACACCCACCTGAACCCAAGACTGGTGTTAACTTCAC
TTTATTCAGCTGTCAAATGGCACTTTAGGCATGTCTTACCTTGGAATCCTTTCAAG
AGTCAATTAACTCTCCCACATCATCCAGAATCTAAAGACCACAGAGTAACTTTCA
AAATATATAATTTTTAACCTCACTGGATTTAAGAACAGTTGGAAAGCTCCAGGTA
TATTTTGAGACTAGTGTCAGTATTACCCTGATAGCAACCAGACAAAGATAACAA
GAAAACTACAGGCCAGAAACTTTTATGAATATAGGTGCAAAGATGCTAAACAAA
ATACTAGCAAACCAAATCCAGCAGCATAGAAAATGGTTTATGTAGCATAGACAA
GTGGGGTTATTCCAGGAATGCAAGGTTCGTTCAATACAAAAATTCCATCAATGTA
ATACAACATATCAATAAAGGACAAAAACTACATGACAACCTTTTTAGATACAGA
AGCATTTGACAAAATTCAATACCCTTTTTTGATTAAAAAAAAAAAAAGACTCAGT
AAGGTAGGAATAGATGGAATATTTCTCAACTTGCAGTATCTATGAAAATCCCACA
GCTAACAACATATTTAATGGTGAAAGGCCTAAAGCTTTCCCACTACAATCAGAA
ACAAGAAAAAGTTGTACACTCTTGCCACTTCTATTTACTTCTATATACTGGATGTG
CTAGCCAGGGCACTTAGGCAGGGAAATCAAATAAAAATAATTCAGATTGGAGAG
GAAACAAAAGTATTTATATTCACAGATGCACAATCTTATATAAAAAATTTTAAAA
ACAAAATCAGTTCAGAATAGCTATGGTATGTAAGGTTAATACACGAATATCAATT
CTTTCTAGATGTACAATGAGTAATCCAAACAAATATTTTTTTTAAAAAAAAGTTG
TTTTATAATAGCATCAAGTGAAAAACATTTAACCCAAAAAAGCCAAGATGTATA
CACTGAAATCTATAAGAACAACATTAAAGGAAATTACAAACCTAAATAAATGGA
AAGACACTCCATGTTTATGGATTAGAAAATTTAATACTGTTAAAATACCACCATA
CTTCAATCTGATGTATTGATTCAACACAAATACTATCAAAATCACAGATGCTTTTT
ATGTAGAAATTAAATAAATCCTAAAATCCAAGTGGACTGCAAG
SEQ ID NO: 12 (rat 1)
AGAACATAGAGAGAGAATCTGATTAGTTTGGGAGTAGTTTATAAATCTTCAACTG
GGCTGGACTCCACGTAATGTCTTTCCAAAAATCAAACCTTTGAATAAATTTTCAA
AAAAAATCTCACTCCACAGGGAAAGTGTACATGTGGAGAAATTGAAGTTTCCGG
TGTTGCTTTAGATTACACATTCTTCCCAATTTCATTCAAAGTTTTTCATCCTGCTGT
TACTAACATTCAGTGCAGAATCTATCAAAAAACAAATCCTTCCAAGGAAATAGTT
TCAAGAGCAAACCTGGCCTGAAAGAACCTGATACCACTGTGAGGCATGACAATA
TGGCCTGAACGAGTCTCAGAAACATTGGTAACCTTCTCAGTTTAGAGATATGGCA
ATCCATTAAGCCTAACACAGATGGATCCCTGGCTGAATATTGGTCAGTCTTTTCT
GTACAACTAGTCTTGAAAGCATTCACTTACCAGCATTGGGCACTGAGTTTTACCA
TGCACATTGGACTTTGCAGAAGAATGATCACTCCCTTGCAGGCTGAATGAGAAA
CAAAGGGTTTGCTTCAAACAGTCTGTAAATATCTCTGAGTAGTCCTTCATTAATT
GTCCTCTCTATGTTACTTGCGTAATGCACCCTGAGTGTTCTTCACTGACAATGTTT
AGGTAAAGTTCAAATGTGATCTATGTAATTGCTTCAAGAGTGAGGCTGTGTTGCC
ATGCATTGGTCTTTTGTACATTTATTGACCTTTCTCACAAGAGAGGGATTTAAACA
TCTGGATGCTGGGAATGGTAGTATAGAAGAGGCAACTGCAAGAGGTATTCTTCTC
AGTGGCATTATAACTTTAGAGCTCTTATACAGTCTCTCACTTTAAAAGTATTCTAT
GTAAAAAGGGCAAACAAGTGCCTTCACATCTTGCTCTTTCCTCCTGTACAGAAGA
CATTCCTCAAAGACTCAAGACAGGGTTCCCATGCATCACTAGAATAGTCTTCAAC
CCTGCATCCCTTCCCTCTTGCAAGTGACCATTCGTAACTCTGAGACAGTGTTGGC
AGCCAAGGTAAGCCATTGTGTTAGGTGATATTAAATTATGTATAGGTATGTTCTA
CCTGGATCTGGATCAATGATGACAACAAAAAGTCATGCAGAGAGATGATGATAA
AAAATCATGCTCAATAGGATTATGCTGAGGCCCATCCAGGGGGACAGGGTTACA
AGCAGTCCTTGTTCTCTTTGAGTACAATTTATGGGACTCTTCTGAAGTCCTCTGAT
GCCCTTGTGCAGTTTCCCCTCACCAGGAATCTGGACACTCTCAGTACTCTTTTGTT
GGCAAACATTGCCTGCTAAGTACCACCTAGTCTCACATTCTACCTGTATGTAAGT
CTGTGAGGCATATCAACTGCTCATGACCCTGCACTTTATGAGGTCTTGCAGTCAT
CTGGCATGCCCTTGTATCCCAGAAAATCACATATCTAAGGTGAGTGAAAAGATC
ATGTCTAATGACTCCCCCTACTACATCTGATAGAGCTAATATCTAATATATACAA
AGAACTCAAGAAGTTAGACACCATAAAATCAAATAACCCTATTAAAAATGGAAT
ATGGAGCTAAACAAAGAATTCTAAACTGAAGAATATCAAATATATGAGAAGTAC
TTAAAAATGTCCAATATCCTTAGTCATCAGGAAAACGCAAATCAAAAAGCCCTG
AGATTCTACCTCACACCAGTGAGAATAACTAAGATGAAAAGCTCAGGGGACAAC
AGATGCTGGTGAGGATGTGGAGAAAGAGAAACACTGGTGGTGGGATTGCAAGCT
GGAACAACCATTCTAGAATGCAGTCTGGAGGTTCCCCAGAAAATTCGACATAGG
AATCCCTGAGGATCCTGCTATACCACTCCTGGGCATATACTGAAAAGATGGTACA
ACATATAATGAAGACTATGCTCACAGTAGCCTTATTTATTATAGCCAGAAGCTGC
AAAGAACCCAGATGTCCTGTAACAG
SEQ ID NO: 13 (rat 2)
AGGTTTATGCAAGGGTTCCCTTGAATCCATAGAATAGCTTTCAACCCTGCAGCCC
TCTGTACTTACTAGTGACCATTCACAATTCCGAGGCAGTGTTGGCAGCCCAGGTT
AACCATGGTGTTAAGTGATACTAAACTCTGGGTGTCCTGCCTGGGTCTGGGTCAA
TGTTGACAACAAAAAGTCATGGACAGAGATGAAGTTATAACAATCATGCTCAAT
GGAATAATGCTGAGGCCCAACCAGGGATACAGGGTTACAAGAAGTCCTTTTTCTT
TTTGTCGACCACTTGCAGGACTCTTCTGAGAACTTCTGATGCCTTTGTGTTGTTTC
CCTTACAAGGAACCTGGACACCTTCAGTTCATGTTTGTTGACAAACACCACCTGA
TAAGTAGCACCTAGTCTCACATTCTACTTGTGCAAGTCTCTGCAAAGCCCACCCA
ATGCTCATGACCCTGTCTCTTCTCATGTCTTGCAGTCATTTAGAATGACCCCTGCA
GCCCAGAGAATCATAGATCTTAGGTAAATGAAAAGATCTTATCTAATGGGTTCAC
CAACTAATGACCTATGCCTCCAATTTTGACCTCTGAATCTCAGCCCATCTAAAGT
TTTTTTTTAATTAAAAACAAAATAACAGAACATGGCAACTAAAGGACAATTCTCA
GTATGTTCACCCCTGTAGAACTGCAGATTTTCATGACATACAGATTTTCCTAACT
ATCAATAAATTTTACACATCAATTTGAGCTACTTGGCATAGGTGGAAGGGGAAA
ACACCTTTCTGTACTTGAATGCCCTTTTTGTTGTTTACAATTTGAGGAGTTGCAGG
ACATCAATACAGGATTCCAAGCCTGTGCCTAGTTTTAAATTTTGCATGACATTGG
AGAGCCTAGCACCAGAGTCTGAATTAGTAAATGAGTAGTACCTATAAATTTCCA
ACTGGCCTGGTCTCCACGTGTATTCACAAAAAGCAAAGCTTTGAGCAAATTTTCA
AAACCTCTTACCACACAGGGAAACAGTACATGTGGAGAACTTGAAGTTTCAGTA
GTTGCCTTGGGTACCATTACCTTTCCCACTTCCTTCACAGTGTTTCATCCTGCTCTT
ACTAAACTTCACTATGGAATTCATCAGGAAACAAATCCTATCAAGGAGATGGTTC
CAGGAACAGGCCTGTTCTGAAGGAACCTGATGTCACTGTGAGACATGAAAACAT
GGGCTGAACCATCTCAGAAACTTTGGTCACCTTCTCAGTATAGTGATGTGGCAAT
CCATTAAACCCTAACACAAATGAATCCATGGCCAAATATTAATCAATCTCTTCTC
TGCCACTGACTCTCTGCAGCATTCACTTACCAGCATTGGGCACTGAGTTTTACGT
ACAGGTTGGACTTTGGAGGGGAATGATCACTCCCTTGCAGAAACAAAAGGTACC
CTACAAACAGTCTCTAAAATTTCTATGTAGTTGTTCACTTATCGTCTTATCCATAT
TACTCAGGAAAGTCAACTTGAGTGTTGGTCCATGACAATATTTAGGCAAAGAAC
AGATGTGATCTGGGCAATTGATTCAAGAGGAAGATGTGAGCCTGTGCTGCAATG
CATTGGCCTTTTCCACTTTCGTTGACATTAATAACGAGATGGGAAGAAACCTGGA
GAATGGGCACGGTAGTATAGAAAAGGCAGTTGCAAGAGGGATTCTGCTCAGAGG
CATCATACGTGTAGAGCTCCTATACAGTCTCTCACATTAAATTATTTTATTTTATT
TATGTTTTTTTTAATTTTTTATTAGCTATATTTCTTTGCTTACATTTCAACTGTTATT
TCCCTTCCCGGTTTCCTGTCCATAAGCCCCCATTCACTCCCTTCCCCCTCCCCCAT
ATGGGTATTCCCCTATACATCCCCTTTACTGCCCCCCCATATTCCCCTGCACTGGG
GGTCCAACCTTAGCAGGACCAAGGGCTTCCCCTTCCACTGGTGCCCCAACAAGGC
TATTATCTGCTACATATGCAGTTGGAGCCCTGGGTAAGTCCATATAAAGTCTTTC
GGTAGTGCTTTAGTCCCTGGAAGCTCTGGTTGGTTGGCATTGTTGTTTTTATGGGG
TTGCAAGCTCCTTCAACCCTTTCCATACTTCCTTTAATTACCCCCAAGGGGGTCCT
GTTCTCAGTTTAGTGGTTTGCTACTAGCATTGACCTCTGTATTAGACATGCTCTAG
ATGTGTCTCTTTTTCTTTTCTTTTTTTATCTTTATTAACTTGGGTATTCCTTATTTAC
ATTTCGATTGTTATTCCCTCTCCCGGTTTCTGGGCCAACATCACCCTAGCCCCTCC
CCCTCCCCTTCTCTGTGGGTGTTCCCCTCCCCATCCTTCCCCCATTACCACCCTCTC
TCCAACAATCCCATTTACTGGGGGTTCAGTCTTGGCAGGACCAACGGCTTCCCCT
TCCACTGCTGCTCTTACTAGGCTATTCTGAATGTGTCTCTTAGGCAGAGGAATGG
ATTCAGAAAATGTGGTACATCTACACAGTGGAGTACTACTCAGCTATCAAAAAC
AATGACTTCATGAAATTCATAGGCAAATGGAATGAACTAGAAAATATCCTGATT
GCTGTAACTCAATCCCAGAAAAACACACTTGGTATGCACTCATTGATAAGTGGAT
ATTAGCCAAAAAGCTCGAATTACATTATTTTATTAAAGAAGGGCAAACAAGTGC
CTTTAAATCTTGATCTTTCCTCCTGTAGAGAAGACATTCTCCAAAGATTCAAGCA
AAGGTTCCCTTGCATCCCTACAAGAGC
SEQ ID NO: 14 (rat 3)
TGGCAAGGGGAACCCAGGAATAAGACTTTAGTTTAGAGATGAAGAAATTGGCGA
TAAATTTTCTGCATCAAAAGACACTCCAAGAATTATTGCCCCAAAGAGAGCTTCG
CAAAATTTTTCACATAACTCTCAGGCCACAGGGAAACTAGCCAGTAGGGTAGAT
TGAAGTTGAAGGTGTGGCTTTGGCTACCTATACCCTTCTATGTGTCTTCACACAG
CTTCATTCTTGATCTTACTAAAATTTACTTCAGTATTCATCAGAAAACAAATGCTA
TCAAGGAGATGGTTCCTCGAGCTCTTGGGACAGAGGAACGTGATGTCAATGGGA
TACTTGCAAACACCAACTGATCTAGTCACACGAATTTTGGTCACATTCTCAGTTT
GATCACATGGCAATCACTGAGTGCTAACGCTCAGGGATTCATGGCCAAAATACTT
GTTGCATTCCTCTGGGCAACAGATACTCTTCAGCATTCACTTACCACCTGTGGGA
ACTGATTGTTACCATGCAGTATTGAAATGGATAGGAATGAACATAACCTAGCAC
ATAAAATCTGAAATGCACAAGTCTCATTAAAATTTGATTAAAGTTTACGTAACTG
TTCGTGTATTGTCCTTCCAATATTACTCAGGTAAATTCACCTTTGCCTTGGTTTCT
GACAATGTTTAGCAATTGAAAGATTTGATCGGTGTATATGACAAAGAGCATGTGT
CATCCCCTTCATCAATGATTTGGTTTTGGGTACCTATTGACCTATTGATCGCTTAG
GGAAGTAACTTTCCATGTGTTGGGAATGTCAGCATGGAAGTGGATGTTGCAAGA
GGGATTCTACTAAGTGGCATCATACATTAGACTTCTTAAACAGCCTCCTAAAATT
AACATGATTGTATGAACTGAAGGGATACAATTGCCCTCACATCTTGCTCTTTCCT
TTTAGAGAGGACAGTCTTCAAATTAAAGCTAGGTCTACCCTTATTCCACACAAGA
GCGCTGTGCCCTTGTACCCTCTGCACTTGCAAGTGAGGATTCAAATTCTGAAGAT
TTGTTGGGAGCCCAGGTAAGCCATGGTGCAGGTGAAATTACATTATTGACAGTTG
TGTCCTACCTGTGTCTGGGTCAATGATGACAACAGAAAGTCAAGAACAGAATGA
TGACATAAAAATCATGCTCAATAGGATTACGCTGAGGCCCAACCAGGGAGCCGG
GGTAACAACACTACTTAGTCTCTTTGAGGACCACTTGCGGGACTCATCTGAGCTG
CTCTGATGACCTTGTGCTCTTTTCCCTCTGTGGGAACCTGGACAATCTTAGTGCAA
CTGTGCTGGCAAACACTGCCTGCCAACTAGCACCTAATCTCATTATCTGCTTGTG
TGCAAGTCTGCAACCCCACCCGGGTCATCATTACCCTCTCTGCTCTGATGTCTTGT
AGTCACCTGGCATCACCCCAGCGGTCCAGTGGATCACTACTGCCAGGTGTGTGAA
ATGATCACGATTAATGGATTTAACTATTCATGAGATATTCCTCAGTATTTTGACCA
ATAATTTCAGCCCATCAAAGGTTTCTTAATTAAAAATAAAGCAATACCAGTCAGA
AATGAATTGCACTTCTAAGTATGTTCTGTCCCTTTGAACTGCACTATTTAAAATGT
AGAGAAATTCCTTTCTACCTGTAGTTCATACAGCCATAAAATTGAATTTCTTGGA
AGAGATCCTAGATGATACTATCTACACAAATGGAGGGATCTGGAATATGAATAT
ACCTTTTGAATTTGGAGTAATTTCAGTCCACACACAGGATCACAAATCCAAGCTG
GTGCACAGAGATCGATTTCCAATGGCAAGGGGATCCCAGGAATAAGACTTTAGT
TTAGAGATGAAGAAATTGGCGATAAATTTTCTGCATCAAAAGACACTCCAAGAA
TTATTGCCCCAAAGAGAGCTTTGCGAAATTTTTCACGTAACTCTCAGGCCACAGG
GAACATAGCCAGTAGGGTAGATTGAAGTTGTAGGTGTGGCTTTGGCTACCAATTC
CCTTCGATGTGTCATCACACTGCTTAATTCTTGATCTTACTAAAGTTTACTTCAGT
ATTCATCAGAAAACAAATGCTATCAATGAGAGAGTTTCAGGAGCTCTTGGGACA
GAAGAACATGATGTCAATGGCATACTTGCAAACACCAACTGATCTAGTCACACG
AATTTTGGTCACATTCTGAGTTTCATCACATGGCAATCACTGAGTGCTAACGCTC
AGGGATTCATGGCCAAAATACTTGTTGCTTTCTTCTGGGCAACTGATTCTCTTCAG
CATTCACTTACCAGCTGTGGGAACTGATTTTTACCATGCAGTATGGAAATGGTGT
GGAATGAACACAGACTAGCACATAAAATTTGAAACGCACACGTCTCTTGAAAAT
TTGATTAAAGTATATGTAACTGTTCGTGTATTGTACTCCCCATATTACTCAGAGAA
ATTCACATTTGTCTTGGTTTCTGACAATGTTTATCAATTGAAAGATTTGATCGGTG
TATATGACAAAGAGCATGTGTCATCCCCTTCATCAATGATTTGGTTTTGGGTACCT
ATTGACCTATTGATCGCTTAGAGAAGTAACTTTCCATGTGTTGGGAATGTCAGCA
TGGAAGAGGATGTTGCAAGCGTGATTCTACTAAGTGGCATCATACATTAGACTTC
TTAAACAGCCTCCTTAAAATTAATATGATTGTATGAACTGAGGGATACAATTGCC
CTCACATCTTGCTCTTTCCTTTTTAGAGAGGACAGTCTTCAAATTCAAGCT

What is claimed is:

1. A composition comprising a Cas13 protein and a guide RNA that targets a Ube3a-ATS transcript.

2. The composition of claim 1, wherein the guide RNA targets a Ube3a-ATS transcript downstream of the coding region of a Snord115 transcript.

3. The composition of claim 1 or 2, wherein the guide RNA targets a Ube3a-ATS transcript downstream of the coding region of a Snord116 transcript.

4. The composition of any of one of claims 1 to 3, wherein the guide RNA targets a Ube3a-ATS transcript upstream of a sequence complementary to a polynucleotide in a gene encoding a protein isoform of UBE3A.

5. The composition of claim 4, wherein the protein isoform of UBE3A comprises SEQ ID NO: 1, SEQ ID NO: 2, or SEQ ID NO: 3.

6. The composition of any one of claims 1 to 5, wherein the guide RNA targets a Ube3a-ATS transcript at a sequence complementary to a polynucleotide sequence in a UBE3A/SNORD region.

7. The composition of claim 6, wherein the polynucleotide sequence in a UBE3A/SNORD region is a sequence within SEQ ID NO: 4, SEQ ID NO: 5, SEQ ID NO: 6, SEQ ID NO: 7, SEQ ID NO: 8, SEQ ID NO: 9, SEQ ID NO: 10, or a complementary strand thereof.

8. The composition of claim 6, wherein the polynucleotide sequence in a UBE3A/SNORD region is a sequence within SEQ ID NO: 4, SEQ ID NO: 5, SEQ ID NO: 6, or SEQ ID NO: 7.

9. The composition of claim 6, wherein the polynucleotide sequence in a UBE3A/SNORD region is a sequence within SEQ ID NO: 11, SEQ ID NO: 12, SEQ ID NO: 13, or SEQ ID NO: 14.

10. The composition of any one of claims 6 to 9, wherein the polynucleotide sequence in a UBE3A/SNORD region is about 21 to about 32 nucleotides long.

11. The composition of any one of claims 1 to 10, wherein the Cas13 protein comprises a Cas13a protein, a Cas13b protein, a Cas13c protein, a Cas13d protein, a Cas13X protein, a Cas13Y protein, or a variant thereof.

12. The composition of any one of claims 1 to 11, wherein the Cas13 protein comprises a Cas13d protein or a variant thereof.

13. The composition of any one of claims 1 to 11, wherein the Cas13 protein comprises a Cas13X protein or a variant thereof.

14. The composition of any one of claims 1 to 13, wherein the Cas13 protein comprises Cas13d-N2V8 or Cas13X-M17V6.

15. The composition of any one of claims 1 to 14, further comprising a pharmaceutically acceptable carrier.

16. A vector comprising a polynucleotide sequence encoding a Cas13 protein and a polynucleotide sequence encoding a guide RNA that targets a Ube3a-ATS transcript.

17. The vector of claim 16, wherein the guide RNA targets a Ube3a-ATS transcript downstream of the coding region of a Snord115 transcript.

18. The vector of claim 16 or 17, wherein the guide RNA targets a Ube3a-ATS transcript downstream of the coding region of a Snord116 transcript.

19. The vector of any of one of claim 16 or 18, wherein the guide RNA targets a Ube3a-ATS transcript upstream of a sequence complementary to a polynucleotide in a gene encoding a protein isoform of UBE3A.

20. The vector of claim 19, wherein the protein isoform of UBE3A comprises SEQ ID NO: 1, SEQ ID NO: 2, or SEQ ID NO: 3.

21. The vector of any one of claims 16 to 20, wherein the guide RNA targets a Ube3a-ATS transcript at a sequence complementary to a polynucleotide sequence in a UBE3A/SNORD region.

22. The vector of claim 21, wherein the polynucleotide sequence in a UBE3A/SNORD region is a sequence within SEQ ID NO: 4, SEQ ID NO: 5, SEQ ID NO: 6, SEQ ID NO: 7, SEQ ID NO: 8, SEQ ID NO: 9, or SEQ ID NO: 10.

23. The vector of claim 22, wherein the polynucleotide sequence in a UBE3A/SNORD region is a sequence within SEQ ID NO: 4, SEQ ID NO: 5, SEQ ID NO: 6, or SEQ ID NO: 7.

24. The vector of claim 21, wherein the polynucleotide sequence in a UBE3A/SNORD region is a sequence within SEQ ID NO: 11, SEQ ID NO: 12, SEQ ID NO: 13, or SEQ ID NO: 14.

25. The vector of any one of claims 21 to 24, wherein the polynucleotide sequence in a UBE3A/SNORD region is about 21 to about 32 nucleotides long.

26. The vector of any one of claims 16 to 25, wherein the Cas13 protein comprises a Cas13a protein, a Cas13b protein, a Cas13c protein, a Cas13d protein, a Cas13X protein, a Cas13Y protein, or a variant thereof.

27. The vector of any one of claims 16 to 26, wherein the Cas13 protein comprises a Cas13d protein or a variant thereof.

28. The vector of any one of claims 16 to 26, wherein the Cas13 protein comprises a Cas13X protein or a variant thereof.

29. The vector of any one of claims 16 to 28, wherein the Cas13 protein comprises Cas13d-N2V8 or Cas13X-M17V6.

30. The vector of any one of claims 16 to 29, wherein the polynucleotide sequence encoding a Cas13 protein is codon optimized for expression in a human cell.

31. The vector of any one of claims 16 to 30, wherein the polynucleotide sequence encoding a Cas13 protein is codon optimized for expression in a rat cell.

32. The vector of any one of claims 16 to 31, wherein the polynucleotide sequence encoding the guide RNA comprises a U6::crRNA cassette.

33. The vector of any one of claims 16 to 32, wherein the vector comprises a promoter element that directs transgene expression to the brain and/or spinal cord.

34. The vector of claim 33, wherein the promoter element is neuron-specific.

35. The vector of claim 33 or 34, wherein the promoter element allows for continuous expression in at least part of the brain and/or spinal cord.

36. The vector of any one of claims 33 to 35, wherein the promoter comprises the promoter from the human SYN1 gene (hSYN1).

37. The vector of any one of claims 16 to 36, wherein the vector is an AAV vector.

38. The vector of claim 37, wherein the AAV vector can cross the blood-brain barrier.

39. The vector of claim 37 or 38, wherein the AAV vector encodes an AAV capsid that allows for enhanced transduction to cells in the central nervous system (CNS).

40. The vector of any one of claims 37 to 39, wherein the AAV vector is AAV9.

41. A cell containing the composition of any one of claims 1 to 15.

42. A cell containing the vector of any one of claims 16 to 40.

43. The cell of claim 41 or 42, wherein the cell is a neuron.

44. The cell of claim 41 or 42, wherein the cell is a stem cell.

45. A method for de-repression of UBE3A expression in a cell, comprising contacting the cell with the composition of any one of claims 1 to 15.

46. A method for de-repression of UBE3A expression in a cell, comprising transfecting or transducing the cell with the vector of any one of claims 16 to 40.

47. A method for cleaving a Ube3a-ATS transcript in a cell, comprising contacting the cell with the composition of any one of claims 1 to 15.

48. A method for cleaving a Ube3a-ATS transcript in a cell, comprising transfecting or transducing the cell with the vector of any one of claims 16 to 40.

49. The method of any one of claims 45 to 48, wherein the cell is a neuron.

50. The method of any one of claims 45 to 48, wherein the cell is a stem cell.

51. A method for treating Angelman Syndrome in a subject in need thereof, comprising administering to the subject a therapeutically effective amount of the composition of any one of claims 1 to 15.

52. A method for treating Angelman Syndrome in a subject in need thereof, comprising administering to the subject a therapeutically effective amount of the vector of any one of claims 16 to 40.

53. A method for treating Angelman Syndrome in a subject in need thereof, comprising administering to the subject a therapeutically effective population of the cell of any one of claims 41 to 44.

54. The method of any one of claims 51 to 53, wherein the therapeutically effective amount is an amount sufficient to de-repress the expression of all three major protein isoforms of UBE3A.

55. The method of any one of claims 51 to 54, wherein the route of administration of the AAV is at least one of intraparenchymal, intranasal, intracerebroventricular, intracisternal, intrathecal, intravenous, intramuscular, via the spinal cord, via the eye, or via the cochlea.

56. The method of any one of claims 51 to 55, wherein the route of administration of the AAV is at least one of intrathecal, intracerebroventricular, or via the intracisterna magna.

57. The method of any one of claims 51 to 56, wherein expression of Cas13 and guide RNA is maintained for at least 16 weeks in the subject.

58. The method of any one of claims 51 to 57, wherein the subject has a mutation that affects the expression or activity of the maternal copy of the UBE3A gene.
